SEQUENCE LISTING (8 sequences)

SEQ ID NO: 1 - DNA, 9171 bp, Homo sapiens
atgagtctagtacttaatgatctgcttatctgctgccgtcaactagaacatgatagagctacagaacgaaagaaagaagttgagaaatttaagcgcctgattcgagatcctgaaacaattaaacatctagatcggcattcagattccaaacaaggaaaatatttgaattgggatgctgtttttagatttttacagaaatatattcagaaagaaacagaatgtctgagaatagcaaaaccaaatgtatcagcctcaacacaagcctccaggcagaaaaagatgcaggaaatcagtagtttggtcaaatacttcatcaaatgtgcaaacagaagagcacctaggctaaaatgtcaagaactcttaaattatatcatggatacagtgaaagattcatctaatggtgctatttacggagctgattgtagcaacatactactcaaagacattctttctgtgagaaaatactggtgtgaaatatctcagcaacagtggttagaattgttctctgtgtacttcaggctctatctgaaaccttcacaagatgttcatagagttttagtggctagaataattcatgctgttaccaaaggatgctgttctcagactgacggattaaattccaaatttttggactttttttccaaggctattcagtgtgcgagacaagaaaagagctcttcaggtctaaatcatatcttagcagctcttactatcttcctcaagactttggctgtcaactttcgaattcgagtgtgtgaattaggagatgaaattcttcccactttgctttatatttggactcaacataggcttaatgattctttaaaagaagtcattattgaattatttcaactgcaaatttatatccatcatccgaaaggagccaaaacccaagaaaaaggtgcttatgaatcaacaaaatggagaagtattttatacaacttatatgatctgctagtgaatgagataagtcatataggaagtagaggaaagtattcttcaggatttcgtaatattgccgtcaaagaaaatttgattgaattgatggcagatatctgtcaccaggtttttaatgaagataccagatccttggagatttctcaatcttacactactacacaaagagaatctagtgattacagtgtcccttgcaaaaggaagaaaatagaactaggctgggaagtaataaaagatcaccttcagaagtcacagaatgattttgatcttgtgccttggctacagattgcaacccaattaatatcaaagtatcctgcaagtttacctaactgtgagctgtctccattactgatgatactatctcagcttctaccccaacagcgacatggggaacgtacaccatatgtgttacgatgccttacggaagttgcattgtgtcaagacaagaggtcaaacctagaaagctcacaaaagtcagatttattaaaactctggaataaaatttggtgtattacctttcgtggtataagttctgagcaaatacaagctgaaaactttggcttacttggagccataattcagggtagtttagttgaggttgacagagaattctggaagttatttactgggtcagcctgcagaccttcatgtcctgcagtatgctgtttgactttggcactgaccaccagtatagttccaggaacggtaaaaatgggaatagagcaaaatatgtgtgaagtaaatagaagcttttctttaaaggaatcaataatgaaatggctcttattctatcagttagagggtgacttagaaaatagcacagaagtgcctccaattcttcacagtaattttcctcatcttgtactggagaaaattcttgtgagtctcactatgaaaaactgtaaagctgcaatgaattttttccaaagcgtgccagaatgtgaacaccaccaaaaagataaagaagaactttcattctcagaagtagaagaactatttcttcagacaacttttgacaagatggactttttaaccattgtgagagaatgtggtatagaaaagcaccagtccagtattggcttctctgtccaccagaatctcaaggaatcactggatcgctgtcttctgggattatcagaacagcttctgaataattactcatctgagattacaaattcagaaactcttgtccggtgttcacgtcttttggtgggtgtccttggctgctactgttacatgggtgtaatagctgaagaggaagcatataagtcagaattattccagaaagccaagtctctaatgcaatgtgcaggagaaagtatcactctgtttaaaaataagacaaatgaggaattcagaattggttccttgagaaatatgatgcagctatgtacacgttgcttgagcaactgtaccaagaagagtccaaataagattgcatctggctttttcctgcgattgttaacatcaaagctaatgaatgacattgcagatatttgtaaaagtttagcatccttcatcaaaaagccatttgaccgtggagaagtagaatcaatggaagatgatactaatggaaatctaatggaggtggaggatcagtcatccatgaatctatttaacgattaccctgatagtagtgttagtgatgcaaacgaacctggagagagccaaagtaccataggtgccattaatcctttagctgaagaatatctgtcaaagcaagatctacttttcttagacatgctcaagttcttgtgtttgtgtgtaactactgctcagaccaatactgtgtcctttagggcagctgatattcggaggaaattgttaatgttaattgattctagcacgctagaacctaccaaatccctccacctgcatatgtatctaatgcttttaaaggagcttcctggagaagagtaccccttgccaatggaagatgttcttgaacttctgaaaccactatccaatgtgtgttctttgtatcgtcgtgaccaagatgtttgtaaaactattttaaaccatgtccttcatgtagtgaaaaacctaggtcaaagcaatatggactctgagaacacaagggatgctcaaggacagtttcttacagtaattggagcattttggcatctaacaaaggagaggaaatatatattctctgtaagaatggccctagtaaattgccttaaaactttgcttgaggctgatccttattcaaaatgggccattcttaatgtaatgggaaaagactttcctgtaaatgaagtatttacacaatttcttgctgacaatcatcaccaagttcgcatgttggctgcagagtcaatcaatagattgttccaggacacgaagggagattcttccaggttactgaaagcacttcctttgaagcttcagcaaacagcttttgaaaatgcatacttgaaagctcaggaaggaatgagagaaatgtcccatagtgctgagaaccctgaaactttggatgaaatttataatagaaaatctgttttactgacgttgatagctgtggttttatcctgtagccctatctgcgaaaaacaggctttgtttgccctgtgtaaatctgtgaaagagaatggattagaacctcaccttgtgaaaaaggttttagagaaagtttctgaaacttttggatatagacgtttagaagactttatggcatctcatttagattatctggttttggaatggctaaatcttcaagatactgaatacaacttatcttcttttccttttattttattaaactacacaaatattgaggatttctatagatcttgttataaggttttgattccacatctggtgattagaagtcattttgatgaggtgaagtccattgctaatcagattcaagaggactggaaaagtcttctaacagactgctttccaaagattcttgtaaatattcttccttattttgcctatgagggtaccagagacagtgggatggcacagcaaagagagactgctaccaaggtctatgatatgcttaaaagtgaaaacttattgggaaaacagattgatcacttattcattagtaatttaccagagattgtggtggagttattgatgacgttacatgagccagcaaattctagtgccagtcagagcactgacctctgtgacttttcaggggatttggatcctgctcctaatccacctcattttccatcgcatgtgattaaagcaacatttgcctatatcagcaattgtcataaaaccaagttaaaaagcattttagaaattctttccaaaagccctgattcctatcagaaaattcttcttgccatatgtgagcaagcagctgaaacaaataatgtttataagaagcacagaattcttaaaatatatcacctgtttgttagtttattactgaaagatataaaaagtggcttaggaggagcttgggcctttgttcttcgagacgttatttatactttgattcactatatcaaccaaaggccttcttgtatcatggatgtgtcattacgtagcttctccctttgttgtgacttattaagtcaggtttgccagacagccgtgacttactgtaaggatgctctagaaaaccatcttcatgttattgttggtacacttataccccttgtgtatgagcaggtggaggttcagaaacaggtattggacttgttgaaatacttagtgatagataacaaggataatgaaaacctctatatcacgattaagcttttagatccttttcctgaccatgttgtttttaaggatttgcgtattactcagcaaaaaatcaaatacagtagaggacccttttcactcttggaggaaattaaccattttctctcagtaagtgtttatgatgcacttccattgacaagacttgaaggactaaaggatcttcgaagacaactggaactacataaagatcagatggtggacattatgagagcttctcaggataatccgcaagatgggattatggtgaaactagttgtcaatttgttgcagttatccaagatggcaataaaccacactggtgaaaaagaagttctagaggctgttggaagctgcttgggagaagtgggtcctatagatttctctaccatagctatacaacatagtaaagatgcatcttataccaaggcccttaagttatttgaagataaagaacttcagtggaccttcataatgctgacctacctgaataacacactggtagaagattgtgtcaaagttcgatcagcagctgttacctgtttgaaaaacattttagccacaaagactggacatagtttctgggagatttataagatgacaacagatccaatgctggcctatctacagccttttagaacatcaagaaaaaagtttttagaagtacccagatttgacaaagaaaacccttttgaaggcctggatgatataaatctgtggattcctctaagtgaaaatcatgacatttggataaagacactgacttgtgcttttttggacagtggaggcacaaaatgtgaaattcttcaattattaaagccaatgtgtgaagtgaaaactgacttttgtcagactgtacttccatacttgattcatgatattttactccaagatacaaatgaatcatggagaaatctgctttctacacatgttcagggatttttcaccagctgtcttcgacacttctcgcaaacgagccgatccacaacccctgcaaacttggattcagagtcagagcactttttccgatgctgtttggataaaaaatcacaaagaacaatgcttgctgttgtggactacatgagaagacaaaagagaccttcttcaggaacaatttttaatgatgctttctggctggatttaaattatctagaagttgccaaggtagctcagtcttgtgctgctcactttacagctttactctatgcagaaatctatgcagataagaaaagtatggatgatcaagagaaaagaagtcttgcatttgaagaaggaagccagagtacaactatttctagcttgagtgaaaaaagtaaagaagaaactggaataagtttacaggatcttctcttagaaatctacagaagtataggggagccagatagtttgtatggctgtggtggagggaagatgttacaacccattactagactacgaacatatgaacacgaagcaatgtggggcaaagccctagtaacatatgacctcgaaacagcaatcccctcatcaacacgccaggcaggaatcattcaggccttgcagaatttgggactctgccatattctttccgtctatttaaaaggattggattatgaaaataaagactggtgtcctgaactagaagaacttcattaccaagcagcatggaggaatatgcagtgggaccattgcacttccgtcagcaaagaagtagaaggaaccagttaccatgaatcattgtacaatgctctacaatctctaagagacagagaattctctacattttatgaaagtctcaaatatgccagagtaaaagaagtggaagagatgtgtaagcgcagccttgagtctgtgtattcgctctatcccacacttagcaggttgcaggccattggagagctggaaagcattggggagcttttctcaagatcagtcacacatagacaactctctgaagtatatattaagtggcagaaacactcccagcttctcaaggacagtgattttagttttcaggagcctatcatggctctacgcacagtcattttggagatcctgatggaaaaggaaatggacaactcacaaagagaatgtattaaggacattctcaccaaacaccttgtagaactctctatactggccagaactttcaagaacactcagctccctgaaagggcaatatttcaaattaaacagtacaattcagttagctgtggagtctctgagtggcagctggaagaagcacaagtattctgggcaaaaaaggagcagagtcttgccctgagtattctcaagcaaatgatcaagaagttggatgccagctgtgcagcgaacaatcccagcctaaaacttacatacacagaatgtctgagggtttgtggcaactggttagcagaaacgtgcttagaaaatcctgcggtcatcatgcagacctatctagaaaaggcagtagaagttgctggaaattatgatggagaaagtagtgatgagctaagaaatggaaaaatgaaggcatttctctcattagcccggttttcagatactcaataccaaagaattgaaaactacatgaaatcatcggaatttgaaaacaagcaagctctcctgaaaagagccaaagaggaagtaggtctccttagggaacataaaattcagacaaacagatacacagtaaaggttcagcgagagctggagttggatgaattagccctgcgtgcactgaaagaggatcgtaaacgcttcttatgtaaagcagttgaaaattatatcaactgcttattaagtggagaagaacatgatatgtgggtattccgactttgttccctctggcttgaaaattctggagtttctgaagtcaatggcatgatgaagagagacggaatgaagattccaacatataaatttttgcctcttatgtaccaattggctgctagaatggggaccaagatgatgggaggcctaggatttcatgaagtcctcaataatctaatctctagaatttcaatggatcacccccatcacactttgtttattatactggccttagcaaatgcaaacagagatgaatttctgactaaaccagaggtagccagaagaagcagaataactaaaaatgtgcctaaacaaagctctcagcttgatgaggatcgaacagaggctgcaaatagaataatatgtactatcagaagtaggagacctcagatggtcagaagtgttgaggcactttgtgatgcttatattatattagcaaacttagatgccactcagtggaagactcagagaaaaggcataaatattccagcagaccagccaattactaaacttaagaatttagaagatgttgttgtccctactatggaaattaaggtggaccacacaggagaatatggaaatctggtgactatacagtcatttaaagcagaatttcgcttagcaggaggtgtaaatttaccaaaaataatagattgtgtaggttccgatggcaaggagaggagacagcttgttaagggccgtgatgacctgagacaagatgctgtcatgcaacaggtcttccagatgtgtaatacattactgcagagaaacacggaaactaggaagaggaaattaactatctgtacttataaggtggttcccctctctcagcgaagtggtgttcttgaatggtgcacaggaactgtccccattggtgaatttcttgttaacaatgaagatggtgctcataaaagatacaggccaaatgatttcagtgcctttcagtgccaaaagaaaatgatggaggtgcaaaaaaagtcttttgaagagaaatatgaagtcttcatggatgtttgccaaaattttcaaccagttttccgttacttctgcatggaaaaattcttggatccagctatttggtttgagaagcgattggcttatacgcgcagtgtagctacttcttctattgttggttacatacttggacttggtgatagacatgtacagaatatcttgataaatgagcagtcagcagaacttgtacatatagatctaggtgttgcttttgaacagggcaaaatccttcctactcctgagacagttccttttagactcaccagagatattgtggatggcatgggcattacgggtgttgaaggtgtcttcagaagatgctgtgagaaaaccatggaagtgatgagaaactctcaggaaactctgttaaccattgtagaggtccttctatatgatccactctttgactggaccatgaatcctttgaaagctttgtatttacagcagaggccggaagatgaaactgagcttcaccctactctgaatgcagatgaccaagaatgcaaacgaaatctcagtgatattgaccagagtttcaacaaagtagctgaacgtgtcttaatgagactacaagagaaactgaaaggagtggaagaaggcactgtgctcagtgttggtggacaagtgaatttgctcatacagcaggccatagaccccaaaaatctcagccgacttttcccaggatggaaagcttgggtgtga

SEQ ID NO: 2 - protein, 3056 aa, Homo sapiens
MSLVLNDLLICCRQLEHDRATERKKEVEKFKRLIRDPETIKHLDRHSDSKQGKYLNWDAVFRFLQKYIQKETECLRIAKPNVSASTQASRQKKMQEISSLVKYFIKCANRRAPRLKCQELLNYIMDTVKDSSNGAIYGADCSNILLKDILSVRKYWCEISQQQWLELFSVYFRLYLKPSQDVHRVLVARIIHAVTKGCCSQTDGLNSKFLDFFSKAIQCARQEKSSSGLNHILAALTIFLKTLAVNFRIRVCELGDEILPTLLYIWTQHRLNDSLKEVIIELFQLQIYIHHPKGAKTQEKGAYESTKWRSILYNLYDLLVNEISHIGSRGKYSSGFRNIAVKENLIELMADICHQVFNEDTRSLEISQSYTTTQRESSDYSVPCKRKKIELGWEVIKDHLQKSQNDFDLVPWLQIATQLISKYPASLPNCELSPLLMILSQLLPQQRHGERTPYVLRCLTEVALCQDKRSNLESSQKSDLLKLWNKIWCITFRGISSEQIQAENFGLLGAIIQGSLVEVDREFWKLFTGSACRPSCPAVCCLTLALTTSIVPGTVKMGIEQNMCEVNRSFSLKESIMKWLLFYQLEGDLENSTEVPPILHSNFPHLVLEKILVSLTMKNCKAAMNFFQSVPECEHHQKDKEELSFSEVEELFLQTTFDKMDFLTIVRECGIEKHQSSIGFSVHQNLKESLDRCLLGLSEQLLNNYSSEITNSETLVRCSRLLVGVLGCYCYMGVIAEEEAYKSELFQKAKSLMQCAGESITLFKNKTNEEFRIGSLRNMMQLCTRCLSNCTKKSPNKIASGFFLRLLTSKLMNDIADICKSLASFIKKPFDRGEVESMEDDTNGNLMEVEDQSSMNLFNDYPDSSVSDANEPGESQSTIGAINPLAEEYLSKQDLLFLDMLKFLCLCVTTAQTNTVSFRAADIRRKLLMLIDSSTLEPTKSLHLHMYLMLLKELPGEEYPLPMEDVLELLKPLSNVCSLYRRDQDVCKTILNHVLHVVKNLGQSNMDSENTRDAQGQFLTVIGAFWHLTKERKYIFSVRMALVNCLKTLLEADPYSKWAILNVMGKDFPVNEVFTQFLADNHHQVRMLAAESINRLFQDTKGDSSRLLKALPLKLQQTAFENAYLKAQEGMREMSHSAENPETLDEIYNRKSVLLTLIAVVLSCSPICEKQALFALCKSVKENGLEPHLVKKVLEKVSETFGYRRLEDFMASHLDYLVLEWLNLQDTEYNLSSFPFILLNYTNIEDFYRSCYKVLIPHLVIRSHFDEVKSIANQIQEDWKSLLTDCFPKILVNILPYFAYEGTRDSGMAQQRETATKVYDMLKSENLLGKQIDHLFISNLPEIVVELLMTLHEPANSSASQSTDLCDFSGDLDPAPNPPHFPSHVIKATFAYISNCHKTKLKSILEILSKSPDSYQKILLAICEQAAETNNVYKKHRILKIYHLFVSLLLKDIKSGLGGAWAFVLRDVIYTLIHYINQRPSCIMDVSLRSFSLCCDLLSQVCQTAVTYCKDALENHLHVIVGTLIPLVYEQVEVQKQVLDLLKYLVIDNKDNENLYITIKLLDPFPDHVVFKDLRITQQKIKYSRGPFSLLEEINHFLSVSVYDALPLTRLEGLKDLRRQLELHKDQMVDIMRASQDNPQDGIMVKLVVNLLQLSKMAINHTGEKEVLEAVGSCLGEVGPIDFSTIAIQHSKDASYTKALKLFEDKELQWTFIMLTYLNNTLVEDCVKVRSAAVTCLKNILATKTGHSFWEIYKMTTDPMLAYLQPFRTSRKKFLEVPRFDKENPFEGLDDINLWIPLSENHDIWIKTLTCAFLDSGGTKCEILQLLKPMCEVKTDFCQTVLPYLIHDILLQDTNESWRNLLSTHVQGFFTSCLRHFSQTSRSTTPANLDSESEHFFRCCLDKKSQRTMLAVVDYMRRQKRPSSGTIFNDAFWLDLNYLEVAKVAQSCAAHFTALLYAEIYADKKSMDDQEKRSLAFEEGSQSTTISSLSEKSKEETGISLQDLLLEIYRSIGEPDSLYGCGGGKMLQPITRLRTYEHEAMWGKALVTYDLETAIPSSTRQAGIIQALQNLGLCHILSVYLKGLDYENKDWCPELEELHYQAAWRNMQWDHCTSVSKEVEGTSYHESLYNALQSLRDREFSTFYESLKYARVKEVEEMCKRSLESVYSLYPTLSRLQAIGELESIGELFSRSVTHRQLSEVYIKWQKHSQLLKDSDFSFQEPIMALRTVILEILMEKEMDNSQRECIKDILTKHLVELSILARTFKNTQLPERAIFQIKQYNSVSCGVSEWQLEEAQVFWAKKEQSLALSILKQMIKKLDASCAANNPSLKLTYTECLRVCGNWLAETCLENPAVIMQTYLEKAVEVAGNYDGESSDELRNGKMKAFLSLARFSDTQYQRIENYMKSSEFENKQALLKRAKEEVGLLREHKIQTNRYTVKVQRELELDELALRALKEDRKRFLCKAVENYINCLLSGEEHDMWVFRLCSLWLENSGVSEVNGMMKRDGMKIPTYKFLPLMYQLAARMGTKMMGGLGFHEVLNNLISRISMDHPHHTLFIILALANANRDEFLTKPEVARRSRITKNVPKQSSQLDEDRTEAANRIICTIRSRRPQMVRSVEALCDAYIILANLDATQWKTQRKGINIPADQPITKLKNLEDVVVPTMEIKVDHTGEYGNLVTIQSFKAEFRLAGGVNLPKIIDCVGSDGKERRQLVKGRDDLRQDAVMQQVFQMCNTLLQRNTETRKRKLTICTYKVVPLSQRSGVLEWCTGTVPIGEFLVNNEDGAHKRYRPNDFSAFQCQKKMMEVQKKSFEEKYEVFMDVCQNFQPVFRYFCMEKFLDPAIWFEKRLAYTRSVATSSIVGYILGLGDRHVQNILINEQSAELVHIDLGVAFEQGKILPTPETVPFRLTRDIVDGMGITGVEGVFRRCCEKTMEVMRNSQETLLTIVEVLLYDPLFDWTMNPLKALYLQQRPEDETELHPTLNADDQECKRNLSDIDQSFNKVAERVLMRLQEKLKGVEEGTVLSVGGQVNLLIQQAIDPKNLSRLFPGWKAWV

SEQ ID NO: 3 - DNA, 7935 bp, Homo sapiens
atgggggaacatggcctggagctggcttccatgatccccgccctgcgggagctgggcagtgccacaccagaggaatataatacagttgtacagaagccaagacaaattctgtgtcaattcattgaccggatacttacagatgtaaatgttgttgctgtagaacttgtaaagaaaactgactctcagccaacctccgtgatgttgcttgatttcatccagcatatcatgaaatcctccccacttatgtttgtaaatgtgagtggaagccatgaggccaaaggcagttgtattgaattcagtaattggatcataacgagacttctgcggattgcagcaactccctcctgtcatttgttacacaagaaaatctgtgaagtcatctgttcattattatttctttttaaaagcaagagtcctgctatttttggggtactcacaaaagaattattacaactttttgaagacttggtttacctccatagaagaaatgtgatgggtcatgctgtggaatggccagtggtcatgagccgatttttaagtcaattagatgaacacatgggatatttacaatcagctcctttgcagttgatgagtatgcaaaatttagaatttattgaagtcactttattaatggttcttactcgtattattgcaattgtgttttttagaaggcaagaactcttactttggcagataggttgtgttctgctagagtatggtagtccaaaaattaaatccctagcaattagctttttaacagaactttttcagcttggaggactaccagcacaaccagctagcacttttttcagctcatttttggaattattaaaacaccttgtagaaatggatactgaccaattgaaactctatgaagagccattatcaaagctgataaagacactatttccctttgaagcagaagcttatagaaatattgaacctgtctatttaaatatgctgctggaaaaactctgtgtcatgtttgaagacggtgtgctcatgcggcttaagtctgatttgctaaaagcagctttgtgccatttactgcagtatttccttaaatttgtgccagctgggtatgaatctgctttacaagtcaggaaggtctatgtgagaaatatttgtaaagctcttttggatgtgcttggaattgaggtagatgcagagtacttgttgggcccactttatgcagctttgaaaatggaaagtatggaaatcattgaggagattcaatgccaaactcaacaggaaaacctcagcagtaatagtgatggaatatcacccaaaaggcgtcgtctcagctcgtctctaaacccttctaaaagagcaccaaaacagactgaggaaattaaacatgtggacatgaaccaaaagagcatattatggagtgcactgaaacagaaagctgaatcccttcagatttcccttgaatacagtggcctaaagaatcctgttattgagatgttagaaggaattgctgttgtcttacaactgactgctctgtgtactgttcattgttctcatcaaaacatgaactgccgtactttcaaggactgtcaacataaatccaagaagaaaccttctgtagtgataacttggatgtcattggatttttacacaaaagtgcttaagagctgtagaagtttgttagaatctgttcagaaactggacctggaggcaaccattgataaggtggtgaaaatttatgatgctttgatttatatgcaagtaaacagttcatttgaagatcatatcctggaagatttatgtggtatgctctcacttccatggatttattcccattctgatgatggctgtttaaagttgaccacatttgccgctaatcttctaacattaagctgtaggatttcagatagctattcaccacaggcacaatcacgatgtgtgtttcttctgactctgtttccaagaagaatattccttgagtggagaacagcagtttacaactgggccctgcagagctcccatgaagtaatccgggctagttgtgttagtggattttttatcttattgcagcagcagaattcttgtaacagagttcccaagattcttatagataaagtcaaagatgattctgacattgtcaagaaagaatttgcttctatacttggtcaacttgtctgtactcttcacggcatgttttatctgacaagttctttaacagaacctttctctgaacacggacatgtggacctcttctgtaggaacttgaaagccacttctcaacatgaatgttcatcttctcaactaaaagcttctgtctgcaagccattccttttcctactgaaaaaaaaaatacctagtccagtaaaacttgctttcatagataatctacatcatctttgtaagcatcttgattttagagaagatgaaacagatgtaaaagcagttcttggaactttattaaatttaatggaagatccagacaaagatgttagagtggcttttagtggaaatatcaagcacatattggaatccttggactctgaagatggatttataaaggagctttttgtcttaagaatgaaggaagcatatacacatgcccaaatatcaagaaataatgagctgaaggataccttgattcttacaacaggggatattggaagggccgcaaaaggagatttggtaccatttgcactcttacacttattgcattgtttgttatccaagtcagcatctgtctctggagcagcatacacagaaattagagctctggttgcagctaaaagtgttaaactgcaaagttttttcagccagtataagaaacccatctgtcagtttttggtagaatcccttcactctagtcagatgacagcacttccgaatactccatgccagaatgctgacgtgcgaaaacaagatgtggctcaccagagagaaatggctttaaatacgttgtctgaaattgccaacgttttcgactttcctgatcttaatcgttttcttactaggacattacaagttctactacctgatcttgctgccaaagcaagccctgcagcttctgctctcattcgaactttaggaaaacaattaaatgtcaatcgtagagagattttaataaacaacttcaaatatattttttctcatttggtctgttcttgttccaaagatgaattagaacgtgcccttcattatctgaagaatgaaacagaaattgaactggggagcctgttgagacaagatttccaaggattgcataatgaattattgctgcgtattggagaacactatcaacaggtttttaatggtttgtcaatacttgcctcatttgcatccagtgatgatccatatcagggcccgagagatatcatatcacctgaactgatggctgattatttacaacccaaattgttgggcattttggctttttttaacatgcagttactgagctctagtgttggcattgaagataagaaaatggccttgaacagtttgatgtctttgatgaagttaatgggacccaaacatgtcagttctgtgagggtgaagatgatgaccacactgagaactggccttcgattcaaggatgattttcctgaattgtgttgcagagcttgggactgctttgttcgctgcctggatcatgcttgtctgggctcccttctcagtcatgtaatagtagctttgttacctcttatacacatccagcctaaagaaactgcagctatcttccactacctcataattgaaaacagggatgctgtgcaagattttcttcatgaaatatattttttacctgatcatccagaattaaaaaagataaaagccgttctccaggaatacagaaaggagacctctgagagcactgatcttcagacaactcttcagctctctatgaaggccattcaacatgaaaatgtcgatgttcgtattcatgctcttacaagcttgaaggaaaccttgtataaaaatcaggaaaaactgataaagtatgcaacagacagtgaaacagtagaacctattatctcacagttggtgacagtgcttttgaaaggttgccaagatgcaaactctcaagctcggttgctctgtggggaatgtttaggggaattgggggcgatagatccaggtcgattagatttctcaacaactgaaactcaaggaaaagattttacatttgtgactggagtagaagattcaagctttgcctatggattattgatggagctaacaagagcttaccttgcgtatgctgataatagccgagctcaagattcagctgcctatgccattcaggagttgctttctatttatgactgtagagagatggagaccaacggcccaggtcaccaattgtggaggagatttcctgagcatgttcgggaaatactagaacctcatctaaataccagatacaagagttctcagaagtcaaccgattggtctggagtaaagaagccaatttacttaagtaaattgggtagtaactttgcagaatggtcagcatcttgggcaggttatcttattacaaaggttcgacatgatcttgccagtaaaattttcacctgctgtagcattatgatgaagcatgatttcaaagtgaccatctatcttcttccacatattctggtgtatgtcttactgggttgtaatcaagaagatcagcaggaggtttatgcagaaattatggcagttctaaagcatgacgatcagcataccataaatacccaagacattgcatctgatctgtgtcaactcagtacacagactgtgttctccatgcttgaccatctcacacagtgggcaaggcacaaatttcaggcactgaaagctgagaaatgtccacacagcaaatcaaacagaaataaggtagactcaatggtatctactgtggattatgaagactatcagagtgtaacccgttttctagacctcataccccaggatactctggcagtagcttcctttcgctccaaagcatacacacgagctgtaatgcactttgaatcatttattacagaaaagaagcaaaatattcaggaacatcttggatttttacagaaattgtatgctgctatgcatgaacctgatggagtggccggagtcagtgcaattagaaaggcagaaccatctctaaaagaacagatccttgaacatgaaagccttggcttgctgagggatgccactgcttgttatgacagggctattcagctagaaccagaccagatcattcattatcatggtgtagtaaagtccatgttaggtcttggtcagctgtctactgttatcactcaggtgaatggagtgcatgctaacaggtccgagtggacagatgaattaaacacgtacagagtggaagcagcttggaaattgtcacagtgggatttggtggaaaactatttggcagcagatggaaaatctacaacatggagtgtcagactgggacagctattattatcagccaaaaaaagagatatcacagctttttatgactcactgaaactagtgagagcagaacaaattgtacctctttcagctgcaagctttgaaagaggctcctaccaacgaggatatgaatatattgtgagattgcacatgttatgtgagttggagcatagcatcaaaccacttttccagcattctccaggtgacagttctcaagaagattctctaaactgggtagctcgactagaaatgacccagaattcctacagagccaaggagcctatcctggctctccggagggctttactaagcctcaacaaaagaccagattacaatgaaatggttggagaatgctggctgcagagtgccagggtagctagaaaggctggtcaccaccagacagcctacaatgctctccttaatgcaggggaatcacgactcgctgaactgtacgtggaaagggcaaagtggctctggtccaagggtgatgttcaccaggcactaattgttcttcaaaaaggtgttgaattatgttttcctgaaaatgaaaccccacctgagggtaagaacatgttaatccatggtcgagctatgctactagtgggccgatttatggaagaaacagctaactttgaaagcaatgcaattatgaaaaaatataaggatgtgaccgcgtgcctgccagaatgggaggatgggcatttttaccttgccaagtactatgacaaattgatgcccatggtcacagacaacaaaatggaaaagcaaggtgatctcatccggtatatagttcttcattttggcagatctctacaatatggaaatcagttcatatatcagtcaatgccacgaatgttaactctatggcttgattatggtacaaaggcatatgaatgggaaaaagctggccgctccgatcgtgtacaaatgaggaatgatttgggtaaaataaacaaggttatcacagagcatacaaactatttagctccatatcaatttttgactgctttttcacaattgatctctcgaatttgtcattctcacgatgaagtttttgttgtcttgatggaaataatagccaaagtatttctagcctatcctcaacaagcaatgtggatgatgacagctgtgtcaaagtcatcttatcccatgcgtgtgaacagatgcaaggaaatcctcaataaagctattcatatgaaaaaatccttagagaagtttgttggagatgcaactcgcctaacagataagcttctagaattgtgcaataaaccggttgatggaagtagttccacattaagcatgagcactcattttaaaatgcttaaaaagctggtagaagaagcaacatttagtgaaatcctcattcctctacaatcagtcatgatacctacacttccatcaattctgggtacccatgctaaccatgctagccatgaaccatttcctggacattgggcctatattgcagggtttgatgatatggtggaaattcttgcttctcttcagaaaccaaagaagatttctttaaaaggctcagatggaaagttctacatcatgatgtgtaagccaaaagatgacctgagaaaggattgtagactaatggaattcaattccttgattaataagtgcttaagaaaagatgcagagtctcgtagaagagaacttcatattcgaacatatgcagttattccactaaatgatgaatgtgggattattgaatgggtgaacaacactgctggtttgagacctattctgaccaaactatataaagaaaagggagtgtatatgacaggaaaagaacttcgccagtgtatgctaccaaagtcagcagctttatctgaaaaactcaaagtattccgagaatttctcctgcccaggcatcctcctatttttcatgagtggtttctgagaacattccctgatcctacatcatggtacagtagtagatcagcttactgccgttccactgcagtaatgtcaatggttggttatattctggggcttggagaccgtcatggtgaaaatattctctttgattctttgactggtgaatgcgtacatgtagatttcaattgtcttttcaataagggagaaacctttgaagttccagaaattgtgccatttcgcctgactcataatatggttaatggaatgggtcctatgggaacagagggtctttttcgaagagcatgtgaagttacaatgaggctgatgcgtgatcagcgagagcctttaatgagtgtcttaaagacttttctacatgatcctcttgtggaatggagtaaaccagtgaaagggcattccaaagcgccactgaatgaaactggagaagttgtcaatgaaaaggccaagacccatgttcttgacattgagcagcgactacaaggtgtaatcaagactcgaaatagagtgacaggactgccgttatctattgaaggacatgtgcattaccttatacaggaagctactgatgaaaacttactatgccagatgtatcttggttggactccatatatgtga

SEQ ID NO: 4 - protein, 2644 aa, Homo sapiens
MGEHGLELASMIPALRELGSATPEEYNTVVQKPRQILCQFIDRILTDVNVVAVELVKKTDSQPTSVMLLDFIQHIMKSSPLMFVNVSGSHEAKGSCIEFSNWIITRLLRIAATPSCHLLHKKICEVICSLLFLFKSKSPAIFGVLTKELLQLFEDLVYLHRRNVMGHAVEWPVVMSRFLSQLDEHMGYLQSAPLQLMSMQNLEFIEVTLLMVLTRIIAIVFFRRQELLLWQIGCVLLEYGSPKIKSLAISFLTELFQLGGLPAQPASTFFSSFLELLKHLVEMDTDQLKLYEEPLSKLIKTLFPFEAEAYRNIEPVYLNMLLEKLCVMFEDGVLMRLKSDLLKAALCHLLQYFLKFVPAGYESALQVRKVYVRNICKALLDVLGIEVDAEYLLGPLYAALKMESMEIIEEIQCQTQQENLSSNSDGISPKRRRLSSSLNPSKRAPKQTEEIKHVDMNQKSILWSALKQKAESLQISLEYSGLKNPVIEMLEGIAVVLQLTALCTVHCSHQNMNCRTFKDCQHKSKKKPSVVITWMSLDFYTKVLKSCRSLLESVQKLDLEATIDKVVKIYDALIYMQVNSSFEDHILEDLCGMLSLPWIYSHSDDGCLKLTTFAANLLTLSCRISDSYSPQAQSRCVFLLTLFPRRIFLEWRTAVYNWALQSSHEVIRASCVSGFFILLQQQNSCNRVPKILIDKVKDDSDIVKKEFASILGQLVCTLHGMFYLTSSLTEPFSEHGHVDLFCRNLKATSQHECSSSQLKASVCKPFLFLLKKKIPSPVKLAFIDNLHHLCKHLDFREDETDVKAVLGTLLNLMEDPDKDVRVAFSGNIKHILESLDSEDGFIKELFVLRMKEAYTHAQISRNNELKDTLILTTGDIGRAAKGDLVPFALLHLLHCLLSKSASVSGAAYTEIRALVAAKSVKLQSFFSQYKKPICQFLVESLHSSQMTALPNTPCQNADVRKQDVAHQREMALNTLSEIANVFDFPDLNRFLTRTLQVLLPDLAAKASPAASALIRTLGKQLNVNRREILINNFKYIFSHLVCSCSKDELERALHYLKNETEIELGSLLRQDFQGLHNELLLRIGEHYQQVFNGLSILASFASSDDPYQGPRDIISPELMADYLQPKLLGILAFFNMQLLSSSVGIEDKKMALNSLMSLMKLMGPKHVSSVRVKMMTTLRTGLRFKDDFPELCCRAWDCFVRCLDHACLGSLLSHVIVALLPLIHIQPKETAAIFHYLIIENRDAVQDFLHEIYFLPDHPELKKIKAVLQEYRKETSESTDLQTTLQLSMKAIQHENVDVRIHALTSLKETLYKNQEKLIKYATDSETVEPIISQLVTVLLKGCQDANSQARLLCGECLGELGAIDPGRLDFSTTETQGKDFTFVTGVEDSSFAYGLLMELTRAYLAYADNSRAQDSAAYAIQELLSIYDCREMETNGPGHQLWRRFPEHVREILEPHLNTRYKSSQKSTDWSGVKKPIYLSKLGSNFAEWSASWAGYLITKVRHDLASKIFTCCSIMMKHDFKVTIYLLPHILVYVLLGCNQEDQQEVYAEIMAVLKHDDQHTINTQDIASDLCQLSTQTVFSMLDHLTQWARHKFQALKAEKCPHSKSNRNKVDSMVSTVDYEDYQSVTRFLDLIPQDTLAVASFRSKAYTRAVMHFESFITEKKQNIQEHLGFLQKLYAAMHEPDGVAGVSAIRKAEPSLKEQILEHESLGLLRDATACYDRAIQLEPDQIIHYHGVVKSMLGLGQLSTVITQVNGVHANRSEWTDELNTYRVEAAWKLSQWDLVENYLAADGKSTTWSVRLGQLLLSAKKRDITAFYDSLKLVRAEQIVPLSAASFERGSYQRGYEYIVRLHMLCELEHSIKPLFQHSPGDSSQEDSLNWVARLEMTQNSYRAKEPILALRRALLSLNKRPDYNEMVGECWLQSARVARKAGHHQTAYNALLNAGESRLAELYVERAKWLWSKGDVHQALIVLQKGVELCFPENETPPEGKNMLIHGRAMLLVGRFMEETANFESNAIMKKYKDVTACLPEWEDGHFYLAKYYDKLMPMVTDNKMEKQGDLIRYIVLHFGRSLQYGNQFIYQSMPRMLTLWLDYGTKAYEWEKAGRSDRVQMRNDLGKINKVITEHTNYLAPYQFLTAFSQLISRICHSHDEVFVVLMEIIAKVFLAYPQQAMWMMTAVSKSSYPMRVNRCKEILNKAIHMKKSLEKFVGDATRLTDKLLELCNKPVDGSSSTLSMSTHFKMLKKLVEEATFSEILIPLQSVMIPTLPSILGTHANHASHEPFPGHWAYIAGFDDMVEILASLQKPKKISLKGSDGKFYIMMCKPKDDLRKDCRLMEFNSLINKCLRKDAESRRRELHIRTYAVIPLNDECGIIEWVNNTAGLRPILTKLYKEKGVYMTGKELRQCMLPKSAALSEKLKVFREFLLPRHPPIFHEWFLRTFPDPTSWYSSRSAYCRSTAVMSMVGYILGLGDRHGENILFDSLTGECVHVDFNCLFNKGETFEVPEIVPFRLTHNMVNGMGPMGTEGLFRRACEVTMRLMRDQREPLMSVLKTFLHDPLVEWSKPVKGHSKAPLNETGEVVNEKAKTHVLDIEQRLQGVIKTRNRVTGLPLSIEGHVHYLIQEATDENLLCQMYLGWTPYM

SEQ ID NO: 5 - DNA, 1494 bp, Homo sapiens
atggtgaggagcaggcaaatgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctacctcatctagaaggagagcaattagtgagacagaagaaaattcagatgaattatctggtgaacgacaaagaaaacgccacaaatctgatagtatttccctttcctttgatgaaagcctggctctgtgtgtaataagggagatatgttgtgaaagaagcagtagcagtgaatctacagggacgccatcgaatccggatcttgatgctggtgtaagtgaacattcaggtgattggttggatcaggattcagtttcagatcagtttagtgtagaatttgaagttgaatctctcgactcagaagattatagccttagtgaagaaggacaagaactctcagatgaagatgatgaggtatatcaagttactgtgtatcaggcaggggagagtgatacagattcatttgaagaagatcctgaaatttccttagctgactattggaaatgcacttcatgcaatgaaatgaatcccccccttccatcacattgcaacagatgttgggcccttcgtgagaattggcttcctgaagataaagggaaagataaaggggaaatctctgagaaagccaaactggaaaactcaacacaagctgaagagggctttgatgttcctgattgtaaaaaaactatagtgaatgattccagagagtcatgtgttgaggaaaatgatgataaaattacacaagcttcacaatcacaagaaagtgaagactattctcagccatcaacttctagtagcattatttatagcagccaagaagatgtgaaagagtttgaaagggaagaaacccaagacaaagaagagagtgtggaatctagtttgccccttaatgccattgaaccttgtgtgatttgtcaaggtcgacctaaaaatggttgcattgtccatggcaaaacaggacatcttatggcctgctttacatgtgcaaagaagctaaagaaaaggaataagccctgcccagtatgtagacaaccaattcaaatgattgtgctaacttatttcccctag

SEQ ID NO: 6 - protein, 497 aa, Homo sapiens
MVRSRQMCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 7 - DNA, 1026 bp, Homo sapiens
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcaggaccagaccagctttcaaaaagaaaattgttaa

SEQ ID NO: 8 - protein, 341 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

(19) United States
(12) Patent Application Publication
(10) Pub. No.: US 2021/0311020 A1
(43) Pub. Date: Oct. 7, 2021

(54) TREATMENT METHODS AND BIOMARKERS FOR MDM2 INHIBITORS

(71) Applicants: ASCENTAGE PHARMA (SUZHOU) CO., LTD., SUZHOU (CN); ASCENTAGE PHARMA GROUP CORP LIMITED, HONG KONG (CN)

(72) Inventors: Yifan ZHAI, SUZHOU (CN); Dajun YANG, SUZHOU (CN); Douglas Dong FANG, SUZHOU (CN); Qiuqiong TANG, SUZHOU (CN); Anthony W. TOLCHER, SAN ANTONIO, TX (US)

(21) Appl. No.: 17/264,293

(22) PCT Filed: Feb. 21, 2020
(86) PCT No.: PCT/CN2020/076094

§ 371 Date: Jan. 28, 2021

Related U.S. Application Data
(60) Provisional application No. 62/809,735, filed on Feb. 24, 2019.
(60) Provisional application No. 62/931,773, filed on Nov. 6, 2019.

(30) Foreign Application Priority Data
Feb. 16, 2020 (CN) 202010094504.X

(51) Int. Cl. G01N 33/50; C12Q 1/6886; A61K 31/407; A61K 45/06

(52) U.S. Cl. CPC G01N 33/5011; C12Q 1/6886; C12Q 2600/106; A61K 45/06; G01N 2800/52; A61K 31/407

(57) ABSTRACT

Provided are biomarkers for predicting the efficacy of MDM2 inhibitors in treating cancer patients. Also provided are compositions, e.g., kits, for evaluating the biomarkers and methods of using the biomarkers to predict a cancer patient's response to the MDM2 inhibitors. Such information can be used in determining prognosis and treatment options for cancer patients.

### FIELD OF THE INVENTION

The present invention relates to treatment methods and biomarkers with MDM2 inhibitors to treat conditions and diseases wherein inhibition of MDM2 and MDM2-related proteins provides a benefit.

### BACKGROUND OF THE INVENTION

MDM2 (Murine Double Minute 2) inhibitors interfere with the binding of MDM2 oncoprotein to the tumor suppressor p53 protein, and serves as a pharmacological p53 activator. Emerging evidence suggests that p53 dysfunction also fuels inflammation and supports tumor immune evasion and, thus, p53 dysfunction serves as an immunological driver of tumorigenesis (Guo G, Cancer Research, 2017; 77(9):2292).

MDM2 and p53 are part of an auto-regulatory feed-back loop (Wu et al., Genes Dev. 7:1126 (1993)). MDM2 is transcriptionally activated by p53, and MDM2, in turn, inhibits p53 activity by at least three mechanisms (Wu et al., Genes Dev. 7:1126 (1993)). First, MDM2 protein directly binds to the p53 transactivation domain, and thereby inhibits p53-mediated transactivation. Second, MDM2 protein contains a nuclear export signal sequence, and upon binding to p53, induces the nuclear export of p53, preventing p53 from binding to the targeted DNAs. Third, MDM2 protein is an E3 ubiquitin ligase and upon binding to p53 is able to promote p53 degradation.

Compound C is a novel, bioavailable, highly potent MDM2 inhibitor.

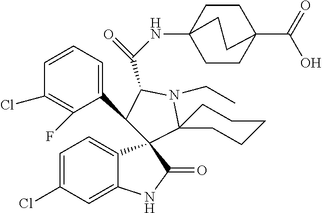 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

Compound C is currently in clinical trials in patients with advanced solid tumor or lymphomas. Given the potency of Compound C, it would be advantageous to further enhance the efficacy of this drug candidate in cancer treatment.

### SUMMARY OF THE INVENTION

Throughout the present disclosure, the articles “a,” “an,” and “the” are used herein to refer to one or to more than one (i.e., to at least one) of the grammatical object of the article. By way of example, “a method” means one method or more than one method.

It has now been found by the inventors of the present application that the administration of an MDM2 inhibitor or a pharmaceutically acceptable salt thereof is particularly effective in patients with certain biomarker characteristics. In particular, it is surprising to find that treatment with MDM2 inhibitor (e.g., Compound C) in such cancer patients can lead to an increase in response rate, more complete regression (CR) responders, delayed tumor growth, as well as conversion of drug-resistant tumors into drug-responding tumors.

Accordingly, provided herein is a method of treating cancer in a subject in need thereof with an MDM2 inhibitor, comprising administering a therapeutically effective amount of the MDM2 inhibitor to the subject, wherein the subject has been identified as having i) an inactivating alteration in ATM, ii) gain in MDM2, or iii) both.

In some embodiments, the inactivating alteration comprises inactivating mutation in ATM, or loss of function or expression in ATM.

In some embodiments, the inactivating mutation in ATM comprises H1380Y, N1983S, c.3154-2 A>G, or any combination thereof.

In some embodiments, the inactivating mutation in ATM is H1380Y.

In some embodiments, the subject identified as having a gain in MDM2 is identified to have a copy number variation (CNV) of >3 in MDM2.

In some embodiments, the subject has been identified as having both H1380Y mutation in ATM and a gain in MDM2.

In some embodiments, the subject has been further identified as having wild-type p53.

In another aspect, provided herein is a method of identifying and/or selecting a patient with cancer for treating with an MDM2 inhibitor, comprising:
a) obtaining a patient sample comprising a cancer cell;
b) detecting the presence of an inactivating alteration in ATM (e.g. H1380Y mutation, N1983S mutation, c.3154-2 A>G, or any combination thereof) in ATM in the sample,
   wherein the presence of the inactivating alteration in ATM identifies a patient who will respond to the treatment with an MDM2 inhibitor.

In some embodiments, the method further comprises
c) detecting copy number variation of MDM2 in the sample to determine whether there is a gain in the MDM2,
wherein the presence of the inactivating alteration in ATM and the gain in MDM2 collectively identifies a patient who will respond to the treatment with an MDM2 inhibitor.

In some embodiments, the cancer cell has been identified as bearing wild-type p53.

In another aspect, provided herein is a method of identifying a subject with cancer as likely to respond to treatment with an MDM2 inhibitor, the method comprising:
a) providing a biological sample from the subject;
b) determining in the biological sample:
   i. if there is deficiency in activity or level of ATM (Ataxia-Telangiectasia Mutated) and/or ATR (Ataxia Telangiectasia and Rad3-related protein); and/or
   ii. if there is gain in activity or level of MDM2;
and
c) identifying the subject as likely to respond to the treatment with an MDM2 inhibitor based on: i) the deficiency in activity or level of ATM and/or ATR, or ii) the gain in activity or level of MDM2, or both i) and ii), found in the biological sample.

In some embodiments, the method further comprising:
d) administering the MDM2 inhibitor to the subject identified as likely to respond to the treatment with an MDM2 inhibitor.

Also provided herein is a method of treating a subject with cancer with an MDM2 inhibitor, the method comprising:
a) determining in a biological sample from the subject:
   i. if there is deficiency in activity or level of ATM and/or ATR; and/or
   ii. if there is gain in activity or level of MDM2; and
b) administering the subject with an MDM2 inhibitor based on: i) the deficiency in the activity or the level of ATM and/or ATR, or ii) the gain in activity or level of MDM2, or both i) and ii), found in the biological sample.

In some embodiments, the step of determining comprises detecting the presence of one or more inactivating mutation in ATM and/or ATR in the biological sample, wherein the presence of the inactivating mutation in ATM and/or ATR is indicative of the deficiency in activity or level of ATM and/or ATR.

In some embodiments, the inactivating mutation comprises translocation, deletion, insertion, substitution, or any combination thereof, that reduces serine/threonine kinase activity of ATM and/or ATR.

In some embodiments, the inactivating mutation in ATM comprises a mutation selected from the group of mutations relative to SEQ ID NO: 2 as listed in FIGS. 1B, 1C and 1D, or c.3154-2A>G relative to SEQ ID NO: 1, or any combination thereof.

In some embodiments, the inactivating mutation in ATM comprises H1380Y, N1983S, N2875S, R2598Q, 1599_1600del, V2716A, K1903fs, V2906I, A1127V, K1101E, Q912*, S2165F, H1083Y, relative to SEQ ID NO: 2, or c.3154-2 A>G relative to SEQ ID NO:1, or any combination thereof.

In some embodiments, the inactivating mutation in ATR comprises K243T, Q1926H, I774fs, K1379N, L1483F, or any combination thereof, relative to SEQ ID NO: 4.

In some embodiments, the step of determining comprises determining if expression level of ATM and/or ATR is reduced in the biological sample relative to a reference level, and wherein the reduced expression level of ATM and/or ATR is indicative of the deficiency in activity or level of ATM and/or ATR.

In some embodiments, the step of determining comprises determining if there is an increase in copy number variation of MDM2 gene, expression level of MDM2 gene product, or activity of MDM2 protein, in the biological sample relative to a reference level, and the increase is indicative of the gain in activity or level of in MDM2.

In some embodiments, a copy number variation (CNV) of >3 in MDM2 is indicative of the gain in activity or level of MDM2.

In some embodiments, an increase of at least 50% in expression level of MDM2 gene product relative to the reference level as measured by RNAseq is indicative of gain in activity or level of MDM2.

In some embodiments, the step of identifying comprises:
c) identifying the subject as likely to respond to the treatment with an MDM2 inhibitor based on: both i) the presence of the inactivating mutation in ATM and/or ATR and ii) the increase in the CNV of the MDM2 gene or the increase in the expression level of the MDM2 gene product, found in the biological sample.

In some embodiments, the step of determining further comprises determining in the biological sample presence or absence of a functional p53.

In some embodiments, the step of determining further comprises determining in the biological sample if p53 is wild-type.

In some embodiments, the step of identifying comprises:
c) identifying the subject as likely to respond to the treatment with an MDM2 inhibitor based on: i) the presence of the inactivating mutation in ATM and/or ATR; ii) the increase in the CNV of the MDM2 gene or the increase in the expression level of the MDM2 gene product; and iii) presence of wild-type p53, found in the biological sample.

In some embodiments, i) the activity or level of ATM and/or ATR, or ii) the gain in activity or level of MDM2, or iii) the presence or absence of a functional p53, is measured by an amplification assay, a hybridization assay, a sequencing assay, or an immunoassay.

Also provided herein is a method of treating a subject with cancer with an MDM2 inhibitor, wherein the subject has been identified as likely to respond to the treatment with the MDM2 inhibitor by any of the method provided herein.

In some embodiments, the biological sample comprises a cancer cell or a non-cancer cell.

In some embodiments, the cancer is solid tumor or hematologic malignancy. In some embodiments, the cancer is gastric cancer, cholangiocarcinoma, lung cancer, melanoma, breast cancer, colon cancer, ovarian cancer, prostate cancer, liver cancer (e.g. hepatocellular carcinoma), bladder cancer, pancreatic cancer, renal cancer, esophageal cancer, head and neck cancer, thyroid cancer, cutaneous squamous cell carcinoma, glioblastoma. neuroblastoma, urinary bladder cancer, hysterocarcinoma, melanoma, osteosarcoma, lymphoma (e.g., mantel cell lymphoma, diffuse large B cell lymphoma), leukemia (e.g., T-cell prolymphocytic leukemia, chronic lymphocytic leukemia, or acute myeloid leukemia), multiple myeloma, uterine cancer, colorectal cancer, lung adenocarcinoma, uterine carcinosarcoma CS, lung squamous cell carcinoma, cervical cancer, esophagus cancer, sarcoma, chromophobe, renal cell carcinoma (RCC), clear cell RCC, papillary RCC, uveal melanoma, testicular germ cell, low grade glioma (LGG), mesothelioma, pheochromocytoma and paraganglioma (PCPG), or thymoma. In some embodiments, the cancer is gastric cancer.

In some embodiments, the MDM2 inhibitor has an IC50 of no more than 1 μM (e.g. no more than 500 nM, 400 nM, 300 nM, 200 nM, 150 nM, 100 nM, 50 nM, 20 nM, 10 nM or 5 nM) in inhibiting the binding of MDM2 to p53 as determined by a fluorescence-polarization MDM2 binding assay.

In some embodiments, the MDM2 inhibitor is selected from the group consisting of idasanutlin (RG7388), RG7112, HDM201, KRT-232, AMG 232, BI907828, SAR-405838 (MI-77301), MK-8242 (SCH 900242), DS3032-b, ALRN-6924 and CGM097; or a pharmaceutically acceptable salt of any of the foregoing.

In some embodiments, the MDM2 inhibitor comprises a compound having the following formula (I):

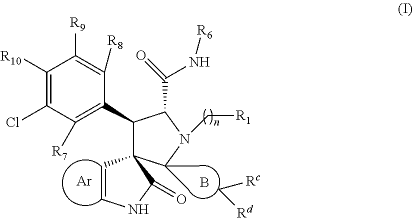

or a pharmaceutically acceptable salt thereof, wherein

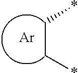

is selected from the group consisting of

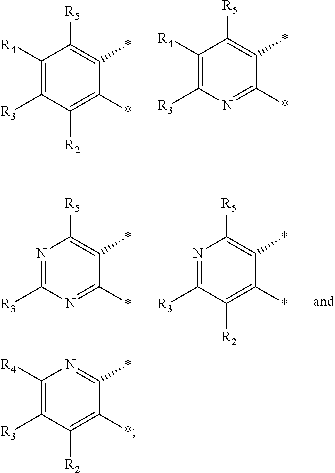

B is a C₄₋₇ carbocyclic ring;
R₁ is H, substituted or unsubstituted C₁₋₄ alkyl, substituted or unsubstituted cycloalkyl, substituted or unsubstituted heterocycloalkyl, OR^{a}, or NR^{a}R^{b};
n is 0, 1, or 2;
R₂, R₃, R₄, R₅, R₇, R₈, R₉, and R₁₀, independently, are selected from the group consisting of H, F, Cl, CH₃, and CF₃;

### R₆ is

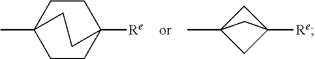

R^{a} is hydrogen or substituted or unsubstituted C₁₋₄ alkyl;
R^{b} is hydrogen or substituted or unsubstituted C₁₋₄ alkyl;
R^{c} and R^{d} are substituents on one carbon atom of ring B, wherein
R^{c} is H, C₁₋₃ alkyl, C₁₋₃ alkylene-OR^{a}, OR^{a}, or halo;
R^{d} is H, C₁₋₃ alkyl, C₁₋₃ alkylene-OR^{a}, OR^{a}, or halo; or
R^{c} and R^{d} are taken together with the carbon to which they are attached to form a 4 to 6-membered Spiro substituent, optionally containing an oxygen atom; and
R^{e} is C(═O)OR^{a}, C(═O)NR^{a}R^{b}, or C(═O)NHSO₂CH₃.

In some embodiments,

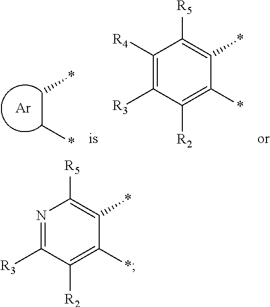

B is

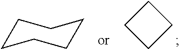

and
R^{c} and R^{d} are respectively F and F, H and H, OH and CH₃, OH and H, CH₃ and CH₃, CH₃ and OH, H and OH, CH₂CH₃ and CH₂CH₃, or CH₂OH and CH₂OH.

In some embodiments,

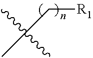

is H, CH₃, or CH₂CH₃.

In some embodiments, R₂ is H; R₃ is halo; R₄ and R₅ are H.

In some embodiments, R₇ is fluoro; each of R₈, R₉, and R₁₀ is H; and R^{e} is —C(═O)OH, C(═O)NH₂, or C(═O)NHSO₂CH₃.

In some embodiments, the MDM2 inhibitor is a compound selected from:

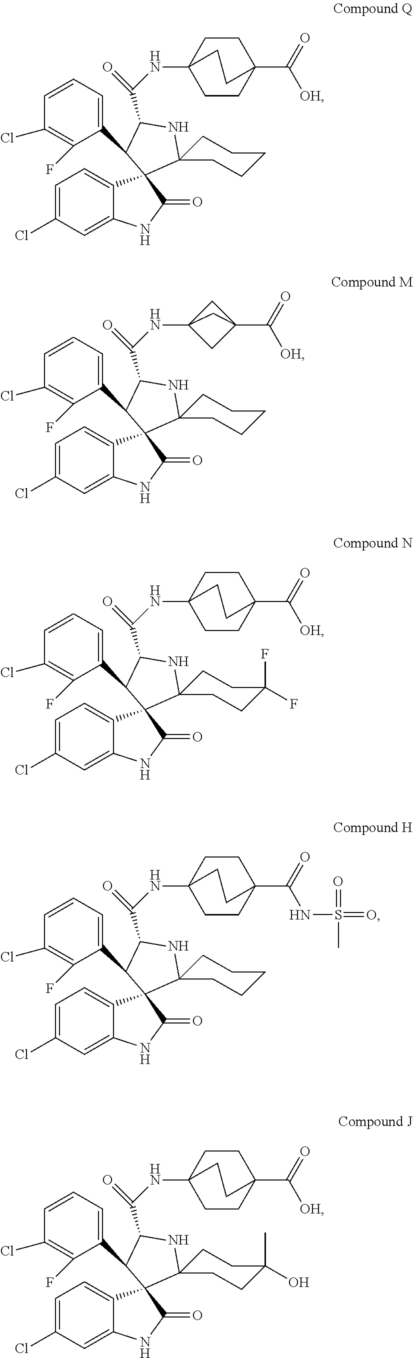 [SMILES: O=C(NC12CCC(C(=O)O)(CC1)CC2)[C@@H]1NC2(CCCCC2)[C@@]2(C(=O)Nc3cc(Cl)ccc32)[C@H]1c1cccc(Cl)c1F ; O=C(NC12CC(C(=O)O)(C1)C2)[C@@H]1NC2(CCCCC2)[C@@]2(C(=O)Nc3cc(Cl)ccc32)[C@H]1c1cccc(Cl)c1F ; O=C(NC12CCC(C(=O)O)(CC1)CC2)[C@@H]1NC2(CCC(F)(F)CC2)[C@@]2(C(=O)Nc3cc(Cl)ccc32)[C@H]1c1cccc(Cl)c1F ; CS(=O)(=O)NC(=O)C12CCC(NC(=O)[C@@H]3NC4(CCCCC4)[C@@]4(C(=O)Nc5cc(Cl)ccc54)[C@H]3c3cccc(Cl)c3F)(CC1)CC2 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21]

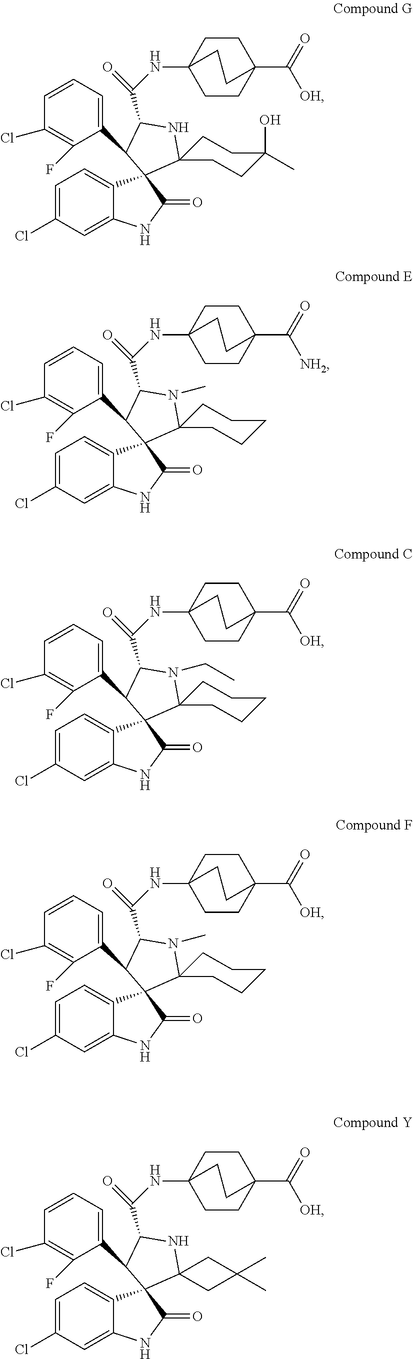 [SMILES: CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21 ; CN1[C@@H](C(=O)NC23CCC(C(N)=O)(CC2)CC3)[C@H](c2cccc(Cl)c2F)[C@]2(C(=O)Nc3cc(Cl)ccc32)C12CCCCC2 ; CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CC1(C)CC2(C1)N[C@@H](C(=O)NC13CCC(C(=O)O)(CC1)CC3)[C@H](c1cccc(Cl)c1F)[C@]21C(=O)Nc2cc(Cl)ccc21]

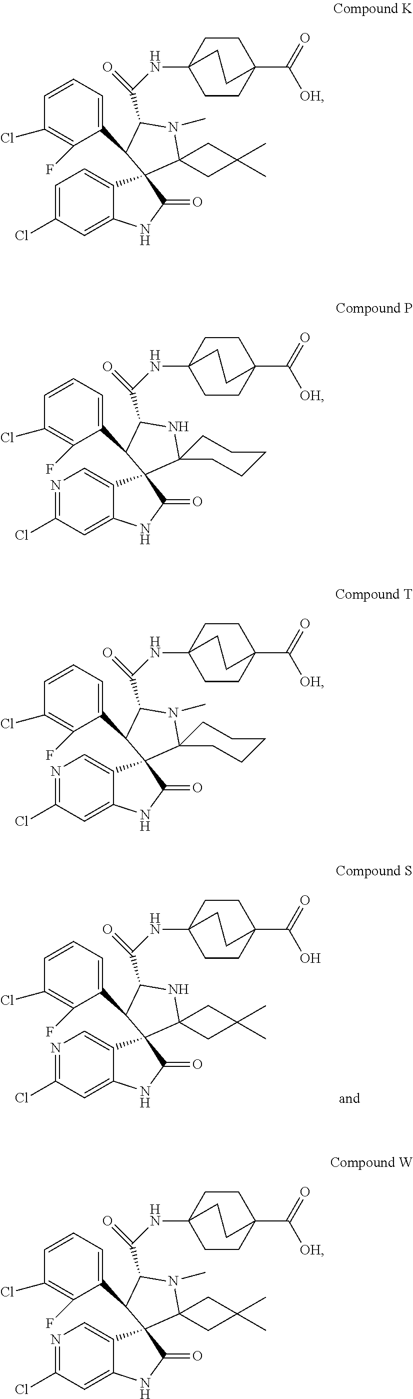 [SMILES: CN1[C@@H](C(=O)NC23CCC(C(=O)O)(CC2)CC3)[C@H](c2cccc(Cl)c2F)[C@]2(C(=O)Nc3cc(Cl)ccc32)C12CC(C)(C)C2 ; O=C(NC12CCC(C(=O)O)(CC1)CC2)[C@@H]1NC2(CCCCC2)[C@@]2(C(=O)Nc3cc(Cl)ncc32)[C@H]1c1cccc(Cl)c1F ; CN1[C@@H](C(=O)NC23CCC(C(=O)O)(CC2)CC3)[C@H](c2cccc(Cl)c2F)[C@]2(C(=O)Nc3cc(Cl)ncc32)C12CCCCC2 ; CC1(C)CC2(C1)N[C@@H](C(=O)NC13CCC(C(=O)O)(CC1)CC3)[C@H](c1cccc(Cl)c1F)[C@]21C(=O)Nc2cc(Cl)ncc21 ; CN1[C@@H](C(=O)NC23CCC(C(=O)O)(CC2)CC3)[C@H](c2cccc(Cl)c2F)[C@]2(C(=O)Nc3cc(Cl)ncc32)C12CC(C)(C)C2]

or a pharmaceutically acceptable salt of the compound.

In some embodiments, the MDM2 inhibitor is

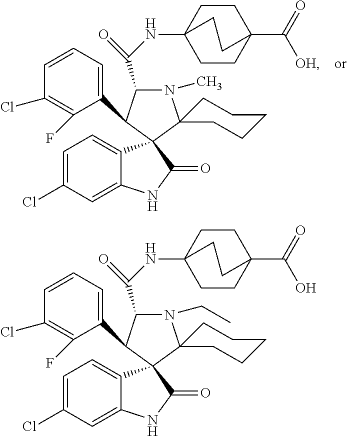 [SMILES: CN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

or a pharmaceutically acceptable salt thereof.

In some embodiments, the MDM2 inhibitor is Compound C or a pharmaceutically acceptable salt thereof.

In some embodiments, the method further comprises further administering an effective amount of one or more additional therapies.

In some embodiments, the one or more additional therapies comprise a radio therapy, a chemotherapy, a targeted cancer therapy, or a therapy with a modulator of an immune checkpoint molecule.

In some embodiments, the one or more additional therapies comprise administering an anti-PD-1 antibody, a Bcl-2 inhibitor, a FAK inhibitor, a MEK inhibitor, or a MET inhibitor.

In some embodiments, the method further comprises further administering an effective amount of one or more additional therapies comprising administering a modulator of an immune checkpoint molecule.

In some embodiments, the MDM2 inhibitor is Compound C or a pharmaceutically acceptable salt thereof and the modulator of an immune checkpoint molecule is an anti-PD-1 antibody.

Also provided herein is a kit for predicting responsiveness of a subject with cancer to treatment with an MDM2 inhibitor, comprising:
a) one or more reagents for detecting presence of one or more inactivating mutations in ATM and/or ATR; or one or more reagents for measuring expression level of ATM and/or ATR; and/or
b) one or more reagents for measuring copy number variation of MDM2, or one or more reagents for measuring expression level of MDM2.

In some embodiments, the kit further comprises one or more reagents for detecting presence or absence of a functional p53. In some embodiments, the kit further comprises one or more reagents for detecting presence of a wild-type p53.

### BRIEF DESCRIPTION OF THE DRAWINGS

FIG. 1A illustrates that inactivation of ATM increases MDM2 function, and decreases p53 Function. FIG. 1B shows exemplary inactivating mutations in ATM. FIG. 1C and FIG. 1D shows exemplary mutations in ATM that may cause deficiency in the activity or level of ATM.

FIG. 2 illustrates that ATM mutations differentiate the responders in mouse trials of Compound C in MDM2^{amp}/p53^{wt}PDX models: from 20% up to 50%.

FIGS. 3A and 3B illustrate activity of Compound C in TP53^{wt}/ATR^{mut} PDX models.

FIG. 4 illustrates activity of Compound C in TP53^{st}/ATM^{wt}/ATR^{wt} PDX models vs. TP53^{wt}/ATM^{mut} or ATR^{mut}PDX models.

FIGS. 5A-5D illustrate that genetically knock-out of ATM gene in A549 cells increases cellular sensitivity to Compound C treatment in vitro. FIG. 5A shows ATM immunoblot on lysates from A549 cells and ATM knock-out (KO) cells. FIG. 5B-5C. show A549 ATM KO cells are more sensitive to Compound C treatment in vitro. FIG. 5D shows Compound C induces more cell apoptosis in A549 ATM KO cells than in parental cells.

FIGS. 6A and 6B illustrate A549 ATM KO cells have higher ROS levels than parental cells, and Compound C treatment results in more ROS induction in ATM KO cells. FIG. 6A shows baseline levels of ROS in A549 parental cells and A549 ATM knock out cells. FIG. 6B shows cells were treated as indicated for 48 hours and ROS level was detected by flow cytometry.

FIG. 7 illustrates exemplary sequences of the biomarkers as provided herein.

### DETAILED DESCRIPTION OF THE INVENTION

### Definitions

In accordance with the present disclosure and as used herein, the following terms are defined with the following meanings, unless explicitly stated otherwise. Unless otherwise defined, all terms of art, notations and other scientific or medical terms or terminology used herein are intended to have the meanings commonly understood by those of skill in the chemical and medical arts.

The term “biomarker” as used here refers to a biological molecule that is a measurable indicator of some biological state or condition. The term “biomarker” used herein is intended to encompass a polynucleotide of interest, or a polypeptide (for example encoded by the polynucleotide of interest). Examples of biomarker provided herein can be a gene (e.g. genomic DNA, cDNA) or a product of the gene such as an mRNA transcribed from the gene, and a protein encoded by the gene. Specific examples of the biomarkers provided herein include ATM, ATR, MDM2, and p53.

“ATM” as used herein is short for ataxia telangiectasia mutated, or ATM serine/threonine kinase. The term ATM is intended to encompass the ATM gene, as well as the ATM gene product (e.g. mRNA, protein). Exemplary sequence of human ATM is available in UniProtKB database under the accession number of Q13315 (ATM-HUMAN), in the GenBank database under the NCBI accession number of AAB65827, and is also published in literatures such as Savitsky K et al, Hum. Mol. Genet. 4:2025-2032(1995), Platzer M. et al, Genome Res. 7:592-605(1997), Bryd P. J. et al, Hum. Mol. Genet. 5:145-149(1996); and Chen G. et al, J. Biol. Chem. 271:33693-33697(1996).

“ATR” as used herein is short for ATM- and Rad3-related, or ATR serine/threonine kinase. The term ATR is intended to encompass the ATR gene, as well as the ATR gene product (e.g. mRNA, protein). Exemplary sequence of human ATR is available in UniProtKB database under the accession number of Q13535 (ATR-HUMAN), in the GenBank database under the NCBI accession number of AAK26749.1, and is also published in literatures such as Bentley et al., EMBO J., 15. 6641-6651(1996) and Cimprich et al., Proc. Natl. Acad. Sci. USA 107:18575-18480 (1996).

“MDM2” as used herein is short for Murine Double Minute 2. The term MDM2 is intended to encompass the MDM2 gene, as well as the MDM2 gene product (e.g. mRNA, protein). Exemplary sequence of human MDM2 is available under the NCBI accession number of ABT17086, ABT17084.1, ABT17085.1, or ABT17083.1.

The term “TP53” and “p53” are used interchangeably herein, and are short for Tumor Protein P53. Alternative names include, e.g., antigen NY-CO-13, phosphorprotein p53, tumor suppressor p53 and cellular tumor antigen p53. Both TP53 and p53 can refer to the protein or the DNA or RNA sequence of the biomarker p53. Exemplary sequence of human p53 is available in UniProtKB database under the accession number of P04637(P53-HUMAN) Exemplary sequence of human p53 is available under the NCBI accession number of AYF55702.1, or AXU92429.1.

The term “level” with respect to a biomarker such as ATM, ATR, MDM2, and/or p53 refers to the amount or quantity of the biomarker of interest present in a sample. Such amount or quantity may be expressed in the absolute terms, i.e., the total quantity of the biomarker in the sample, or in the relative terms, i.e., the concentration or percentage of the biomarker in the sample. Level of a biomarker can be measured at DNA level (for example, as represented by the amount or quantity or copy number of the gene in a chromosomal region), at RNA level (for example as mRNA amount or quantity), or at protein level (for example as protein or protein complex amount or quantity).

The term “activity” with respect to a biomarker such as ATM, ATR, MDM2, and/or p53 refers to the biological activity (e.g. catalytic or regulatory ability) of the proteins as described here.

As used herein, “likelihood” and “likely” with respect to response of a subject to a treatment is a measurement of how probable the therapeutic response is to occur in the subject. It may be used interchangeably with “probability”. Likelihood refers to a probability that is more than speculation, but less than certainty. Thus, a therapeutic response is likely if a reasonable person using common sense, training or experience concludes that, given the circumstances, a therapeutic response is probable. In one embodiment, the term “likelihood” and “likely” denotes a chance in percent of how probable a therapeutic response is to occur. In some embodiments, a subject with cancer identified as “likely to respond” refers to a subject with cancer who has more than 30% chance, more than 40% chance, more than 50% chance, more than 60% chance, more than 70% chance, more than 80% chance, more than 90% chance of responding to the treatment with an MDM2 inhibitor.

The term “responsive” or “responsiveness” as used in the context of a subject's therapeutic response to a cancer therapy, are used interchangeably and refer to a beneficial response of a subject to a treatment as opposed to unfavorable responses, i.e. adverse events. In a subject, beneficial response can be expressed in terms of a number of clinical parameters, including loss of detectable tumor (complete response), decrease in tumor size and/or cancer cell number (partial response), tumor growth arrest (stable disease), enhancement of anti-tumor immune response, possibly resulting in regression or rejection of the tumor; relief, to some extent, of one or more symptoms associated with the tumor; increase in the length of survival following treatment; and/or decreased mortality at a given point of time following treatment. Continued increase in tumor size and/or cancer cell number and/or tumor metastasis is indicative of lack of beneficial response to treatment, and therefore decreased responsiveness.

As used herein, “cancer” is a generic name for a wide range of cellular malignancies characterized by unregulated growth, lack of differentiation, and the potential or ability to invade local tissues and metastasize. These neoplastic malignancies affect, with various degrees of prevalence, every tissue and organ in the body. Cancer involves presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone or may circulate in the blood stream as independent cells, such as leukemic cells.

As used herein, a “pharmaceutically acceptable” component is one that is suitable for use with humans and/or animals without undue adverse side effects (such as toxicity, irritation, and allergic response) commensurate with a reasonable benefit/risk ratio.

The terms “determining”, “measuring” and “detecting” can be used interchangeably and refer to both quantitative and semi-quantitative determinations.

The term “hybridizing” refers to the binding, duplexing or pairing of at least partially complementary strands of nucleic acid molecules. A nucleic acid strand can specifically hybridize to a target nucleic acid strand when there is sufficient degree of complementarity to avoid non-specific binding to non-target nucleic acid sequences.

The term “nucleic acid” and “polynucleotide” are used interchangeably and refer to a polymeric form of nucleotides of any length, either deoxyribonucleotides or ribonucleotides, or analogs thereof. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA, nucleic acid probes, and primers of any sequence. The nucleic acid molecule may be linear or circular.

The term “complementarity” refers to the ability of basepairing between a nucleic acid sequence and another nucleic acid sequence via either traditional Watson-Crick or other non-traditional types. Complementarity can be partial or total. Partial complementarity occurs when one or more nucleic acid bases is not matched according to the base pairing rules. A percent complementarity indicates the percentage of nucleic acid base in a nucleic acid molecule which can form basepairs (e.g., Watson-Crick base pairing) with a second nucleic acid sequence (e.g., 5, 6, 7, 8, 9, 10 base pairing out of 10 bases being 50%, 60%, 70%, 80%, 90%, and 100% complementary).

The term “prognose” or “prognosing” as used herein refers to the prediction or forecast of the future course or outcome of a disease or condition.

In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

The term “treating” or “treatment” of cancer as used herein, unless otherwise indicated, means reversing, alleviating, inhibiting the progress of, or preventing, either partially or completely, the growth of tumors, tumor metastases, or other cancer-causing or neoplastic cells in a subject.

As used herein, “co-administration” or “combination therapy” is understood as administration of two or more active agents using separate formulations or a single pharmaceutical formulation, or consecutive administration in any order such that, there is a time period while both (or all) active agents simultaneously exert their biological activities. It is contemplated herein that one active agent (e.g., an MDM2 inhibitor) can improve the activity of a second agent, for example, can sensitize target cells, e.g., cancer cells, to the activities of the second agent. Co-administration does not require that the agents are administered at the same time, at the same frequency, or by the same route of administration.

The terms “administer”, “administering” or “administration” include any method of delivery of a pharmaceutical composition or agent into a subject's system or to a particular region in or on a subject. In certain embodiments, the agent is delivered orally, or parenterally. In certain embodiments, the agent is delivered by injection or infusion, or delivered topically including transmucosally. In certain embodiments, the agent is delivered by inhalation. In certain embodiments of the invention, an agent is administered by parenteral delivery, including, intravenous, intramuscular, subcutaneous, intramedullary injections, as well as intrathecal, direct intraventricular, intraperitoneal, intranasal, or intraocular injections. In one embodiment, the agent may be administered by injecting directly to a tumor. In some embodiments, the agent may be administered by intravenous injection or intravenous infusion. In certain embodiments, the agent can be administered by continuous infusion. In certain embodiments, administration is not oral. In certain embodiments, administration is systemic. In certain embodiments, administration is local. In some embodiments, one or more routes of administration may be combined, such as, intravenous and intratumoral, or intravenous and peroral, or intravenous and oral, or intravenous and topical, or intravenous and transdermal or transmucosal. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, continuous infusion, etc.

As used herein, the term “subject” refers to a human or any non-human animal or mammal (e.g., mouse, rat, rabbit, dog, cat, cattle, swine, sheep, horse or primate). In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective amount” means the amount of a pharmaceutical agent that that produces some desired local or systemic therapeutic effect at a reasonable benefit/risk ratio applicable to any treatment. When administered for preventing a disease, the amount is sufficient to avoid or delay onset of the disease. A therapeutically effective amount or an effective amount need not be curative or prevent a disease or condition from ever occurring. In certain embodiments, a therapeutically-effective amount of a pharmaceutical agent will depend on its therapeutic index, solubility, and the like.

“Preventing” or “prevention” refers to a reduction in risk of acquiring a disease or disorder. Prevention does not require that the disease or condition never occur, or recur, in the subject.

In all occurrences in this application where there are a series of recited numerical values, it is to be understood that any of the recited numerical values may be the upper limit or lower limit of a numerical range. It is to be further understood that the invention encompasses all such numerical ranges, i.e., a range having a combination of an upper numerical limit and a lower numerical limit, wherein the numerical value for each of the upper limit and the lower limit can be any numerical value recited herein. Ranges provided herein are understood to include all values within the range. For example, 1-10 is understood to include all of the values 1, 2, 3, 4, 5, 6, 7, 8, 9, and 10, and fractional values as appropriate. Similarly, ranges delimited by “at least” are understood to include the lower value provided and all higher numbers.

As used herein, “about” is understood to include within three standard deviations of the mean or within standard ranges of tolerance in the specific art. In certain embodiments, about is understood a variation of no more than 0.5.

The articles “a” and “an” are used herein to refer to one or more than one (i.e. to at least one) of the grammatical object of the article. By way of example, “an element” means one element or more than one element.

The term “including” is used herein to mean, and is used interchangeably with, the phrase “including but not limited to”. Similarly, “such as” is used herein to mean, and is used interchangeably, with the phrase “such as but not limited to”.

The term “or” is used inclusively herein to mean, and is used interchangeably with, the term “and/or,” unless context clearly indicates otherwise.

I. Biomarkers for Predicting Responsiveness to Treatment with MDM2 Inhibitor

The methods a described herein are based, in part, on the discovery of biomarkers whose level and/or activity and/or mutation is predictive of responsiveness of a subject with cancer to treatment with MDM2 inhibitors. MDM2 inhibitors have been described previously as an anti-cancer therapeutic agent (See, e.g, U.S. Pat. No. 9,745,314, the entire contents of which are incorporated herein by reference), and are being evaluated in humans as mono-therapy or in combination with standard of care chemotherapy agents for treatment of diseases and conditions wherein inhibition of MDM2 and MDM2-related proteins activity provides a benefit.

Described herein are methods of identifying a subject with cancer as likely to respond to an MDM2 inhibitor, methods of treating a subject with cancer with an MDM2 inhibitor, and kits for predicting responsiveness of a subject with cancer to an MDM2 inhibitor. Biomarkers found to be useful for the methods provided herein include, ATM, ATR, MDM2 and/or p53.

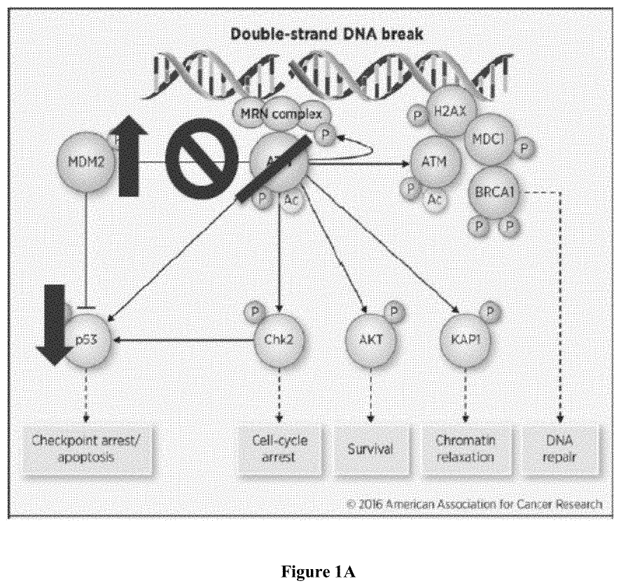

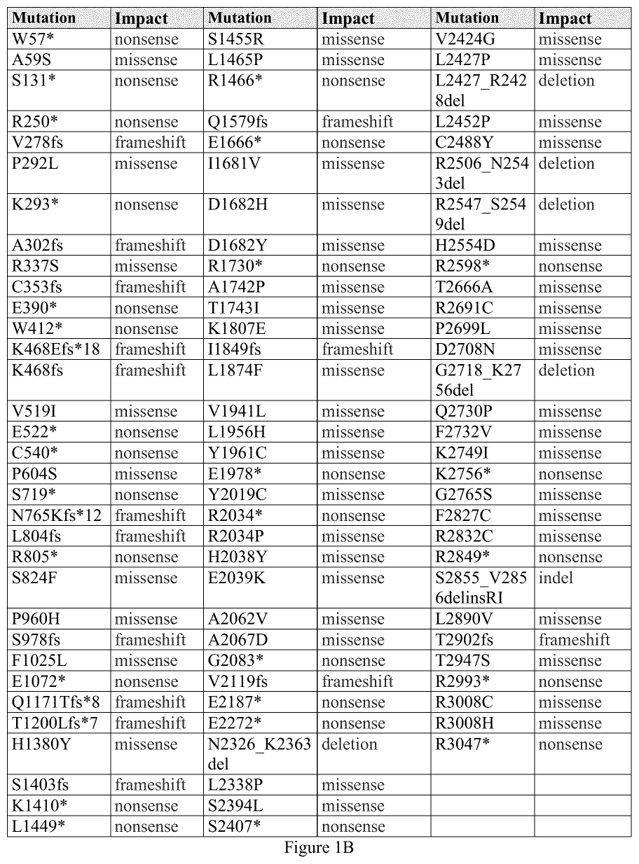

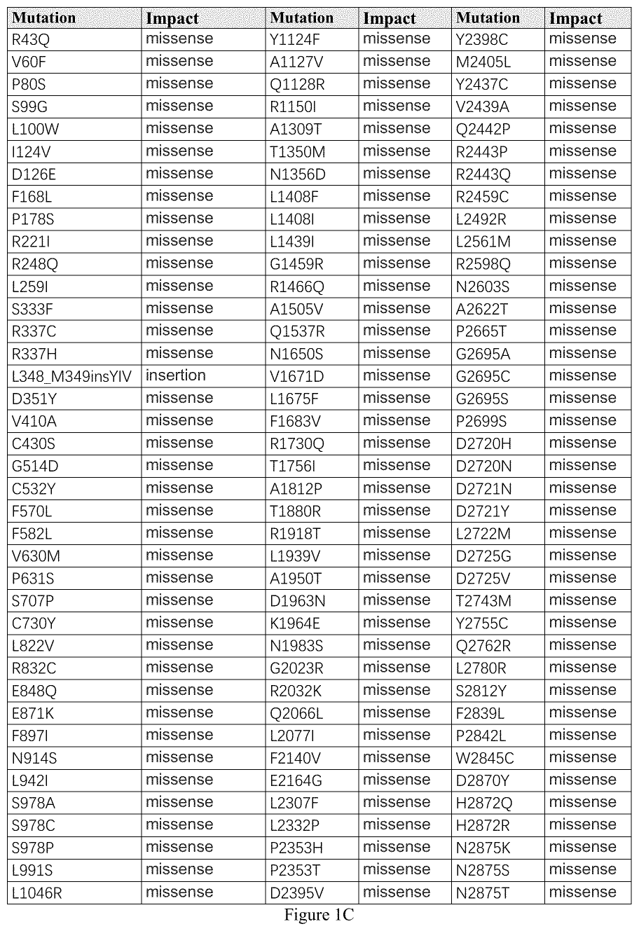

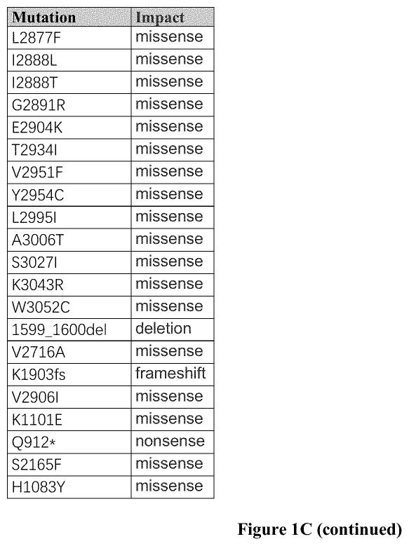

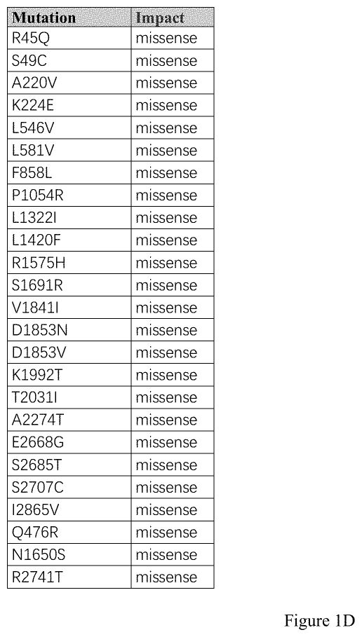

ATM has been suggested as a major post translational regulator of MDM2 (see, for example, Cheng Q et al, EMBO J, 28: 3857-3867 (2009); and Maya R et al, Gene Dev. 15: 1067-1077 (2001)). ATM plays a central role in the repair of DNA double-strand breaks (DSB). ATM deficiency increases genomic instability through loss of DSB DNA repair. In other words, inactivating alteration in ATM (e.g. ATM mutations) may lead to similar effects induced by MDM2 overexpression. As summarized in the schematic FIG. 1A, inactivation of ATM increases MDM2 function, and decreases p53 function.

ATM used as the biomarker herein can be ATM protein as well as a polynucleotide (e.g. DNA or RNA) encoding the ATM protein. In certain embodiments, the gene of ATM comprises a gene sequence of SEQ ID NO: 1. In certain embodiments, the protein of ATM comprises an amino acid sequence of SEQ ID NO: 2.

ATR is highly homologous to ATM and regulates cell response to DNA damage (see, for example, Blackford A N, et al., Mol. Cell, 66:801-817 (2017)). ATR differs from ATM in that its activity is increased by different genotoxic stresses; ATR responds to agents (including ionizing radiation) that cause bulky adducts on double or single strand DNA. However, ATR acts in a way similar to ATM that directly phosphorylates a suite of target proteins, e.g. p53, chk1, chk2, c-Abl and the like (see, for example EP patent No. 1617875B1).

ATR used as the biomarker herein can be ATR protein as well as a polynucleotide (e.g. DNA or RNA) encoding the ATR protein. In certain embodiments, the gene of ATR comprises a gene sequence of SEQ ID NO: 3. In certain embodiments, the protein of ATR comprises an amino acid sequence of SEQ ID NO: 4.

MDM2 is transcriptionally activated by p53 and MDM2, in turn, inhibits p53 activity by at least three mechanisms (Wu et al., Genes Dev. 7:1126 (1993)), including inhibition of p53-mediated transactivation, prevention of p53 from binding to the targeted DNAs, and promotion of p53 degradation. MDM2 are found to be frequently overexpressed in some cancers, and can enhance the tumorigenic potential and resistance to apoptosis through abrogation of p53 function.

MDM2 used as the biomarker herein can be MDM2 protein as well as a polynucleotide (e.g. DNA or RNA) encoding the MDM2 protein. In certain embodiments, the gene of MDM2 comprises a gene sequence of SEQ ID NO: 5. In certain embodiments, the protein of MDM2 comprises an amino acid sequence of SEQ ID NO: 6.

p53 is a transcription factor capable of regulating a number of genes that regulate e.g. cell cycle and apoptosis. p53 protein is controlled by MDM2. By binding to N-Terminal p53, MDM2 inhibits p53 transactivation. In addition, MDM2, as E3 ubiquitin ligase, also targets p53 to proteosomal cytosol degradation. Blocking p53-MDM2 interaction therefore can reduce the negative regulation on p53 function, and enable p53 to mediate its downstream functions. Presence of functional p53 is therefore suggested to be beneficial to response to treatment with MDM2 inhibitors.

The term “functional p53”, as used herein, refers to wild-type p53 and mutant or allelic variants of p53 that retain at least about 5% of the activity of wild-type p53, e.g., at least about 10%, about 20%, about 30%, about 40%, about 50%, about 60%, about 70%, or more of wild-type p53 activity.

p53 used as the biomarker herein can be p53 protein as well as a polynucleotide (e.g. DNA or RNA) encoding the p53 protein. The gene encoding p53 in certain embodiments is referred to as TP53 in the present disclosure. In certain embodiments, the gene of p53 comprises a gene sequence of SEQ ID NO: 7. In certain embodiments, the protein of p53 comprises an amino acid sequence of SEQ ID NO: 8.

### II. Methods for Patient Identification, Treatment Guidance and Prognosis

In one aspect, provided herein is a method of identifying a subject with cancer as likely to respond to treatment with an MDM2 inhibitor. In certain embodiments, the method comprising: providing a biological sample from the subject; determining in the biological sample: if there is deficiency in activity or level of ATM and/or ATR; and/or if there is gain in activity or level of MDM2; and identifying the subject as likely to respond to the treatment with an MDM2 inhibitor based on: i) the deficiency in activity or level of ATM and/or ATR, or ii) the gain in activity or level of MDM2, or both i) and ii), found in the biological sample.

In certain embodiments, the method of identifying a subject with cancer as likely to respond to treatment with MDM2 inhibitor further comprises administering the MDM2 inhibitor to the subject identified as likely to respond to the treatment with an MDM2 inhibitor.

In another aspect, provided herein is a method of selecting a subject with cancer for the treatment with an MDM2 inhibitor. In certain embodiments, the method comprising: providing a biological sample from the subject; determining in the biological sample: if there is deficiency in activity or level of ATM and/or ATR; and/or if there is gain in activity or level of MDM2; and selecting the subject for the treatment with an MDM2 inhibitor based on: i) the deficiency in activity or level of ATM and/or ATR, or ii) the gain activity or level in MDM2, or both i) and ii), found in the biological sample.

In certain embodiments, the method of selecting a subject with cancer for the treatment with an MDM2 inhibitor further comprises administering the MDM2 inhibitor to the selected subject.

In another aspect, provided herein is a method of predicting responsiveness of a subject with cancer to the treatment with an MDM2 inhibitor. In certain embodiments, the method comprises: providing a biological sample from the subject; determining in the biological sample: if there is deficiency in activity or level of ATM and/or ATR; and/or if there is gain in activity or level of MDM2; and predicting the subject as highly responsive to the treatment with an MDM2 inhibitor based on: i) the deficiency in activity or level of ATM and/or ATR, or ii) the gain activity or level of in MDM2, or both i) and ii), found in the biological sample.

In another aspect, provided herein is a method of treating a subject with cancer with an MDM2 inhibitor. In certain embodiments, the method comprising: determining in a biological sample from the subject if there is deficiency in activity or level of ATM and/or ATR; and/or if there is gain in activity or level of MDM2; and administering the subject with an MDM2 inhibitor based on: i) the deficiency in the activity or the level of ATM and/or ATR, or ii) the gain in MDM2, or both i) and ii), found in the biological sample.

In certain embodiments of the methods provided herein, the step of determining further comprises determining in the biological sample if p53 is a functional p53 (e.g. wild-type p53).

i. Sample Preparation

In certain embodiments, the methods provided herein comprises providing a biological sample from the subject.

Any biological sample suitable for conducting the methods provided herein can be obtained from the subject. As used herein, “biological sample” refers to a biological specimen taken by sampling from a subject, optionally with additional processing. In certain embodiments, the sample can be a biological sample comprising cancer cells, or non-cancer cells. For example, non-cancer cells can be from the same tissue or organ as the cancer cells are also found. In some embodiments, the biological sample is a fresh or archived sample obtained from a tumor tissue, e.g., by a tumor biopsy or fine needle aspirate. In some embodiments, the sample can be any biological fluid containing cancer cells or non-cancer cells (e.g. peripheral blood mononuclear cells (PBMC)). The collection of a sample from a subject is performed in accordance with the standard protocol generally followed by hospital or clinics, such as during a biopsy. Examples of a biological sample include without limitation, bodily fluid, such as blood, plasma, serum, urine, vaginal fluid, uterine or vaginal flushing fluids, plural fluid, ascetic fluid, cerebrospinal fluid, saliva, sweat, tears, sputum, bronchioalveolar lavage fluid, etc., and tissues, such as biopsy tissue (e.g. biopsied bone tissue, bone marrow, breast tissue, gastroinstestinal tract tissue, lung tissue, liver tissue, prostate tissue, brain tissue, nerve tissue, meningeal tissue, renal tissue, endometrial tissue, cervical tissue, lymph node tissue, muscle tissue, or skin tissue), a paraffin embedded tissue. In a further embodiment, a biological sample comprises cells, tissue, blood, plasma, serum, urine, mouthwash, stool, saliva, and any combination thereof. In a further embodiment, a biological sample is blood, plasma, serum, or urine. In a preferred embodiment, a biological sample is blood. In another preferred embodiment, a biological sample is tumor tissue.

In certain embodiments, the sample can be further processed by a desirable method for determining the activity or level of the at least one biomarker.

In certain embodiments, the method further comprises isolating or extracting cancer cell (such as circulating tumor cell) from the biological fluid sample (such as peripheral blood sample) or the tissue sample obtained from the subject. The cancer cells can be separated by immunomagnetic separation technology such as that available from Immunicon (Huntingdon Valley, Pa.).

In certain embodiments, a tissue sample can be processed to perform in situ hybridization. For example, the tissue sample can be paraffin-embedded before fixing on a glass microscope slide, and then deparaffinized with a solvent, typically xylene.

In certain embodiments, the method further comprises isolating the nucleic acid from the sample, if RNA or DNA level of the biomarker is to be measured. Various methods of extraction are suitable for isolating the DNA or RNA from cells or tissues, such as phenol and chloroform extraction, and various other methods as described in, for example, Ausubel et al., Current Protocols of Molecular Biology (1997) John Wiley & Sons, and Sambrook and Russell, Molecular Cloning: A Laboratory Manual 3^{rd} ed (2001).

Commercially available kits can also be used to isolate RNA, including for example, the NucliSens extraction kit (Biomerieux, Marcy l'Etoile, France), QIAamp™ mini blood kit, Agencourt Genfind™, Rneasy® mini columns (Qiagen), PureLink® RNA mini kit (Thermo Fisher Scientific), and Eppendorf Phase Lock Gels™. A skilled person can readily extract or isolate RNA or DNA following the manufacturer's protocol.

ii. Determination, Measurement and Detection of the Biomarkers

In certain embodiments, the methods provided herein comprise determining in the biological sample if there is deficiency in activity or level of ATM and/or ATR. As surprisingly discovered by the inventors, deficiency in activity or level of ATM and/or ATR is associated with the likelihood of responsiveness of a subject with cancer to treatment with MDM2 inhibitors provided herein.

As used herein, “deficiency” or “deficient” refers to insufficiency in activity or level, and can include, for example, being less than normal activity or level, or being absent or null in activity or level. For example, deficiency in activity or level of ATM and/or ATR can result in ATM and/or ATR having no or less than normal function, or an absence of or reduced expression level of ATM and/or ATR in a biological sample.

In certain embodiments, the deficiency in activity or level of ATM and/or ATR can be indicated by the presence of the inactivating mutation in ATM and/or ATR. Accordingly, to determine if there is deficiency in activity or level of ATM and/or ATR in the biological sample, the methods provided herein can comprise the step of detecting the presence of one or more inactivating mutation in ATM and/or ATR in the biological sample.

The term “inactivating mutation,” as used herein with respect to a biomarker, refers a mutation that results in at least partial (or complete) loss of function or activity of the gene or of the gene product of the biomarker, or results in a nonfunctional gene or gene product of the biomarker. For example, the activity of the affected gene or gene product of the biomarker would be significantly lower than wild-type counterpart or even be eliminated. In certain embodiments, an inactivating mutation in ATM and/or ATR may be a translocation, deletion, insertion, substitution or any combination thereof, which reduces the biological activity of ATM and/or ATR. In certain embodiments, the inactivating mutation reduces serine/threonine kinase activity of ATM and/or ATR.

As used herein, a “substitution” is a mutation that exchanges one nucleobase for another in a polynucleotide sequence, or that substitutes one amino acid residue for another in a polypeptide sequence. Substitution in a polynucleotide sequence can: 1) change a codon to one that encodes a different amino acid residue, and therefore will cause change in amino acid sequence in the protein produced, or 2) change to a codon that encodes the same amino acid residue thereby causing no change in the protein produced; or 3) change an amino-acid-coding codon to a single “stop” codon and cause an incomplete protein (an incomplete protein is usually nonfunctional). Substitution in a polypeptide sequence can be denoted as AnB, where “n” is a number indicating the n^{th} amino acid residue in the polypeptide sequence, “A” is the amino acid residue at the n^{th} residue in the wild-type polypeptide sequence, and “B” is the mutated amino acid residue at the n^{th} residue. When the mutated residue is shown as “*”, it means a mutation leading to a nonsense codon in a nucleotide sequence that results in a truncated, incomplete polypeptide. For instance, “H1380Y” denotes that the 1380^{th} amino acid residue Histidine (H) is changed to a Tyrosine (Y); “Q912*” denotes that the nucleotides encoding amino acid residue 912 (Glutamine, Q) is changed to a stop codon and the resultant polypeptide is truncated. For another instance, “c. 3154-2 A>G” relative to SEQ ID NO: 1 denotes the A to C substitution at a position (in an intron) of 2 nucleotides upstream the nucleotide residue 3154 of SEQ ID NO: 1, which affects the splice acceptor site and causes frameshift.

As used herein, an “insertion” is a mutation in which one or more extra nucleobase pairs are inserted into a place in a polynucleotide sequence, or in which one or more amino acid residue is inserted into a polypeptide sequence. For instance, “L348_M349insYIV” denotes an insertion of the amino acid sequence YIV between the amino acids residue 348 (Leucine, L) and 349 (Methionine, M).

As used herein, a “deletion” is a mutation in which one or more nucleobase pairs are lost or deleted from a polynucleotide sequence, or in which one or more amino acid residue are deleted from a polypeptide sequence. Deletion in a polypeptide is designated by “del” after the amino acid residue number (s) flanking the deletion site. For instance, “1599_1600del” denotes that amino acid residues 1599-1600 are deleted. For another instance, “S2855_V2856delinsRI” denotes a deletion of two amino acids from amino acids residues 2855 (Serine, S) to 2856 (Valine, V), combined with the insertion of an arginine (R) and an isoleucine (I) at the same site.

As used herein, a “translocation” refers to a type of chromosomal abnormality resulted from the exchange of genetic materials between two non-homologous chromosomes. A translocation may be either balanced or unbalanced; a balanced translocation results in no gain or loss of material, while an unbalanced translocation may result in trisomy or monosomy of a particular chromosome segment. Chromosomal translocations are typically seen in cases of leukemia, like, for instance, in acute myeloid leukemia.

In certain embodiments, insertion or deletion in a polynucleotide sequence may cause frame shift, which changes the reading frame of the codons and results in a completely different translated gene product from the original. This often generates truncated proteins that result in loss of function. Frame shift in a polypeptide is denoted by “AnBfs*m”, indicating a shift in the reading frame starting at the n^{th} amino acid residue and terminating at the m^{th} residues downstream that causes a premature termination of the protein, where “A” and “B” have the same meaning as described above. For instance, “K1903fs” denotes a frame shifting changes with the amino acid residue 1903 (Lysine, K) as the first affected amino acid residue. For another instance, “K468Efs*18” denotes a frame shifting starting at the amino acid residue 468 (Lysine, K) as the first affected amino acid residue and terminating 18 residues downstream.

Inactivating mutation in ATM and/or ATR has been identified in many types of cancers or tumors. For instance, inactivating mutations in both ATM alleles are associated with T-cell polymphocytic leukemia; ATM mutations, such as truncating mutations or missense mutations within the region of the gene encoding the kinase domain, are observed in mantel cell lymphoma; loss of heterozygosity at ATM locus are common in B-cell chronic lymphocyctic leukemia (See, for example, Choi M, Mol Cancer Ther; 15(8); 1781-91.(2016)); truncating mutations in A10 repeat tract of ATR gene are described in endometrial cancer and are associated with biologic aggressivenss (Zighelboim I., J. Clin. Oncol. 27:3091-3096 (2009); and deletion of nucleotides in exon 33 of ATR that leads to putative truncated ATR lacking several functional domains is identified in breast cancer (Durocher, F., BMC Cancer, 6: (2006).

Numerous mutations in ATM gene have been identified to date. For example, more than 2750 mutations in ATM have been identified, as published in Catalogue of Somatic Mutations in Cancer (COSMIC) database which is available from the following weblink: (https://cancer.sanger.ac.uk/cosmic/gene/analysis?ln=ATM#variants). ATM mutations have also been reported in different cancers, for example, in Boultwood J., J Clin Pathol, 54:512-526 (2001), Choi M, et al, Mol Cancer Ther, 15:1781-1791 (2016), Wan Y, et al, Blood, 121:4627-4634(2013), Bullrich F, et al, Cancer Red, 59:24-27 (1999), Roberts N.J., et al, Cancer Discov, 2:41-46 (2012), Shen L, et al, Mol Biol Rep, 39:5719-5725 (2012), and Dombemowsky S I, et al, J Clin Oncol, 26:3057-3062 (2008).

Similarly, numerous mutations in ATR gene have been identified, and more than 1652 mutations in ATR have been published in COSMIC database available from the following weblink: (https://cancer.sanger.ac.uk/cosmic/gene/analysis?ln=ATR#variants). ATR mutations have also been reported in different cancers, for example, in Durocher F., et al., BMC Cancer, 6:230 (2006), Tanaka A, et al, Am J Hum Genet, 90:511-517 (2012), Heikkinen K, et al, Breast Cancer Res, 7:R495-R501 (2005), Stephens P, et al, Nature Genetics 37:590-2 (2005), Sjoblom T, et al, Science, 314: 268-74 (2006) and Zighelboim I, et al, J Clin Oncol, 27:3091-3096 (2009).

It is to be understood that the present disclosure is not limited to any specific ATM or ATR mutations. Any inactivating mutations in ATM or ATR can be useful in the present disclosure. In some embodiments, inactivating mutations in ATM include, without limitation, one or more mutations in FIG. 1B. In some embodiments, inactivating mutations in ATM comprises a mutation selected from the group of mutations relative to SEQ ID NO: 2 as listed in FIGS. 1B, 1C and 1D, or c. 3154-2A>G relative to SEQ ID NO: 1, or any combination thereof.

In some embodiments, inactivating mutations in ATM include, without limitation, H1380Y, N1983S, N2875S, R2598Q, 1599_1600del, V2716A, K1903fs, V2906I, A1127V, K1101E, Q912*, S2165F, or H1083Y relative to SEQ ID NO: 2, or c.3154-2 A>G relative to SEQ ID NO: 1, or any combination thereof.

Examples of inactivating mutations in ATR include, without limitation, K243T, Q1926H, I774fs, K1379N, L1483F, or any combination thereof, relative to SEQ ID NO: 4.

In certain embodiments, the deficiency in activity or level of ATM and/or ATR can be indicated by the expression level or copy number of ATM and/or ATR in the biological sample. Accordingly, to determine if there is deficiency in activity or level of ATM and/or ATR in the biological sample, the methods provided herein can comprise the step of determining if expression level or copy number of ATM and/or ATR is reduced in the biological sample relative to a reference level.

In certain embodiments, the methods of the present disclosure include measuring expression level or gene copies of ATM and/or ATR. Without wishing to be bound by any theory, it is found that in some cases, ATM mutations can lead to a reduction or loss of ATM protein expression, and in some other cases, hypermethylation of the ATM promoter may also result in decreased protein levels.

In certain embodiments, the methods provided herein may comprise or further comprise determining in the biological sample if there is gain in activity or level of MDM2.

The term “gain” with respect to a gene or a gene product (such as MDM2) refers to an increase in the amount or activity of the gene or its product as compared to a reference level, i.e. a reference level that does not have such a gain. For example, a gain can be found in copy number (i.e. amplification) of the MDM2 gene, in expression level of MDM2 gene product, or in the function/activity of MDM2 protein. “Copy number” as used herein refers to the number of copies of a particular gene or a particular genomic sequence in the genome of an individual. “Copy number variation”, or “CNV” refers to the variation in the number of copies of a particular gene or a particular DNA sequence from one individual to another individual. For example, although genes are thought to occur in two copies per genome, some genes or genomic sequences are found to be present in one, three, or more than three copies, or even missing (i.e. 0 copy), in different individuals.

In certain embodiments, the methods of the present disclosure include measuring copy number variation of MDM2, or measuring expression level of MDM2.

In certain embodiments, the methods provided herein further comprise determining in the biological sample presence or absence of a functional p53 (e.g. wild-type p53 or TP53 gene). In certain embodiments, the methods of the present disclosure include determining in the biological sample if p53 is wild-type.

The biomarkers ATM, ATR, MDM2 and/or p53 provided herein are intended to encompass different forms including mRNA, protein and also DNA (e.g. genomic DNA). Therefore, the level and/or activity of these biomarkers can be measured with RNA (e.g. mRNA), protein or DNA (e.g. genomic DNA) of the respective biomarker. Similarly, mutation status and/or wild-type status of the biomarkers can also be measured with DNA (e.g. genomic DNA), RNA (e.g. mRNA), or protein (for example by measuring for an altered protein product encoded by the mutated gene).

Mutation status of a biomarker at DNA or RNA level can be measured by any methods known in the art, for example, without limitation, an amplification assay, a hybridization assay, or a sequencing assay. Mutation status at protein level can be measured by any methods known in the art, for example, without limitation, immunoassays.

Expression level of a biomarker at DNA or RNA level can be measured by any methods known in the art, for example, without limitation, an amplification assay, a hybridization assay, or a sequencing assay. Expression level of a biomarker at protein level can be measured by any methods known in the art, for example, without limitation, immunoassays.

Activity level of a biomarker can be measured by a suitable functional assay known in the art, for example, without limitation, by a phosphorylation assay.

These methods are well-known in the art, and are described in detail below as exemplary illustration.

Amplification Assay

A nucleic acid amplification assay involves copying a target nucleic acid (e.g. DNA or RNA), thereby increasing the number of copies of the amplified nucleic acid sequence. Amplification may be exponential or linear. Exemplary nucleic acid amplification methods include, but are not limited to, amplification using the polymerase chain reaction (“PCR”, see U.S. Pat. Nos. 4,683,195 and 4,683,202; PCR Protocols: A Guide To Methods And Applications (Innis et al., eds, 1990)), reverse transcriptase polymerase chain reaction (RT-PCR), quantitative real-time PCR (qRT-PCR); quantitative PCR, such as TaqMan®, nested PCR, ligase chain reaction (See Abravaya, K., et al., Nucleic Acids Research, 23:675-682, (1995), branched DNA signal amplification (see, Urdea, M. S., et al., AIDS, 7 (suppl 2):S11-S14, (1993), amplifiable RNA reporters, Q-beta replication (see Lizardi et al., Biotechnology (1988) 6: 1197), transcription-based amplification (see, Kwoh et al., Proc. Natl. Acad. Sci. USA (1989) 86: 1173-1177), boomerang DNA amplification, strand displacement activation, cycling probe technology, self-sustained sequence replication (Guatelli et al., Proc. Natl. Acad. Sci. USA (1990) 87:1874-1878), rolling circle replication (U.S. Pat. No. 5,854,033), isothermal nucleic acid sequence based amplification (NASBA), and serial analysis of gene expression (SAGE).

In some embodiments, to measure the mRNA level of the biomarker, the target RNA of the biomarker is reverse transcribed to cDNA before the amplification. Various reverse transcriptases may be used, including, but not limited to, MMLV RT, RNase H mutants of MMLV RT such as Superscript and Superscript II (Life Technologies, GIBCO BRL, Gaithersburg, Md.), AMV RT, and thermostable reverse transcriptase from Thermus thermophilus. For example, one method which may be used to convert RNA to cDNA is the protocol adapted from the Superscript II Preamplification system (Life Technologies, GIBCO BRL, Gaithersburg, Md.; catalog no. 18089-011), as described by Rashtchian, A., PCR Methods Applic., 4:S83-S91, (1994).

In certain embodiments, the expression level of RNA (e.g. mRNA) or the copy number variation of DNA of the biomarkers is quantified after the nucleic acid amplification assay. For example, the amplified products can be separated on an agarose gel and stained with ethidium bromide followed by detection and quantification using standard gel electrophoresis methods. Alternatively, the amplified products can be integrally labeled with a suitable detectable label (e.g. a radio- or fluorescence nucleotide) and then visualized using x-ray film or under the appropriate stimulating spectra.

In certain embodiments, the expression level of RNA (e.g. mRNA) or the copy number variation of DNA of the biomarkers is quantified during the nucleic acid amplification assay, which is also known as real-time amplification or quantitative amplification. Methods of quantitative amplification are disclosed in, e.g., U.S. Pat. Nos. 6,180,349; 6,033,854; and 5,972,602, as well as in, e.g., Gibson et al., Genome Research (1996) 6:995-1001; DeGraves, et al., Biotechniques (2003) 34(1): 106-10, 112-5; Deiman B, et al., Mol Biotechnol. (2002) 20(2): 163-79. Quantification is usually based on the monitoring of the detectable signal representing copies of the template in cycles of an amplification (e.g., PCR) reaction. Detectable signals can be generated by intercalating agents (e.g. SYBR GREEN™ and SYBR GOLD™) or labeled primer or labeled probes used during the amplification.

In certain embodiments, the labeled primer or labeled probe comprise a detectable label comprising a fluorophore. In certain embodiments, the labeled primer or labeled probe may further comprise a quencher substance. Presence of both a fluorophore and a quencher substance (“dual labeled”) in one primer or probe could be helpful to provide for a self-quenching probe such as a TaqMan (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635). In an intact primer or probe, the quencher substance and the fluorophore are in close proximity, such that when the fluorophore is excited by irradiation, it transfers energy to the quencher substance in the same probe via fluorescence resonance energy transfer (FRET), thereby does not emit a signal.

In a quantitative amplification assay (such as real-time PCR), the expression level of RNA (e.g. mRNA) or the copy number variation of DNA of the biomarkers can be quantified using methods known in the art. For example, during the amplification, the fluorescence signal can be monitored and calculated during each PCR cycle. The threshold cycle, or Ct value can be further calculated. Ct value is the cycle at which fluorescence intersects a predetermined value. The Ct can be correlated to the initial amount of nucleic acids or number of starting cells using a standard curve. A standard curve is constructed to correlate the differences between the Ct values and the logarithmic level of the measured biomarker.

As a quality control measure, the expression level or copy number variation of an internal control biomarker may be measured. The skilled artisan will understand that an internal control biomarker can be inherently present in the sample and its expression level or copy number variation can be used to normalize the measured expression level or copy number variation of the biomarkers of interest, to offset any difference in the absolute amount of the sample.

Hybridization Assay

Nucleic acid hybridization assays use probes to hybridize to the target nucleic acid, thereby allowing detection of the target nucleic acid. Non-limiting examples of hybridization assay include Northern blotting, Southern blotting, in situ hybridization, microarray analysis, and multiplexed hybridization-based assays.

In certain embodiments, the probes for hybridization assay are detectably labeled. In certain embodiments, the nucleic acid-based probes for hybridization assay are unlabeled. Such unlabeled probes can be immobilized on a solid support such as a microarray, and can hybridize to the target nucleic acid molecules which are detectably labeled.

In certain embodiments, hybridization assays can be performed by isolating the nucleic acids (e.g. RNA or DNA), separating the nucleic acids (e.g. by gel electrophoresis) followed by transfer of the separated nucleic acid on suitable membrane filters (e.g. nitrocellulose filters), where the probes hybridize to the target nucleic acids and allows detection. See, for example, Molecular Cloning: A Laboratory Manual, J. Sambrook et al., eds., 2nd edition, Cold Spring Harbor Laboratory Press, 1989, Chapter 7. The hybridization of the probe and the target nucleic acid can be detected or measured by methods known in the art. For example, autoradiographic detection of hybridization can be performed by exposing hybridized filters to photographic film. Densitometric scanning of the photographic films exposed by the hybridized filters provides an accurate measurement of the target nucleic acid levels. Computer imaging systems can also be used to quantify the level of the biomarker.

In some embodiments, hybridization assays can be performed on microarrays. Microarrays provide a method for the simultaneous measurement of the levels of large numbers of target nucleic acid molecules. The target nucleic acids can be RNA, DNA, cDNA reverse transcribed from mRNA, or chromosomal DNA. The target nucleic acids can be allowed to hybridize to a microarray comprising a substrate having multiple immobilized nucleic acid probes arrayed at a density of up to several million probes per square centimeter of the substrate surface. The RNA or DNA in the sample is hybridized to complementary probes on the array and then detected by laser scanning. Hybridization intensities for each probe on the array are determined and converted to a quantitative value representing relative levels of the RNA or DNA. See, U.S. Pat. Nos. 6,040,138, 5,800,992 and 6,020,135, 6,033,860, and 6,344,316.

Techniques for the synthesis of these arrays using mechanical synthesis methods are described in, e.g., U.S. Pat. No. 5,384,261. Although a planar array surface is often employed the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device. Useful microarrays are also commercially available, for example, microarrays from Affymetrix, from Nano String Technologies, QuantiGene 2.0 Multiplex Assay from Panomics.

In certain embodiments, hybridization assays can be in situ hybridization assay. In situ hybridization assay is useful to detect the presence of copy number variation (e.g. increase or amplification) at the locus of the biomarker of interest (e.g. ATM, ATR and/or MDM2). Probes useful for in situ hybridization assay can be locus specific probes, which hybridize to a specific locus on a chromosome to detect the presence or absence of a specific locus of interest (e.g. ATM, ATR and/or MDM2). Other types of probes may also be useful, for example, chromosome enumeration probes (e.g. hybridizable to a repeat sequence region in a chromosomal of interest to indicate presence or absence of the entire chromosome), and chromosome arm probes (e.g. hybridizable to a chromosomal region and indicate the presence or absence of an arm of a specific chromosome). Methods for use of unique sequence probes for in situ hybridization are described in U.S. Pat. No. 5,447,841, incorporated herein by reference. Probes can be viewed with a fluorescence microscope and an appropriate filter for each fluorophore, or by using dual or triple band-pass filter sets to observe multiple fluorophores. See, e.g., U.S. Pat. No. 5,776,688 to Bittner, et al., which is incorporated herein by reference. Any suitable microscopic imaging method can be used to visualize the hybridized probes, including automated digital imaging systems. Alternatively, techniques such as flow cytometry can be used to examine the hybridization pattern of the probes.

Sequencing Methods

Sequencing methods allow determination of the nucleic acid sequence of the target nucleic acid, and can also permit enumeration of the sequenced target nucleic acid, thereby measures the level of the target nucleic acid. Examples of sequence methods include, without limitation, RNA sequencing, pyrosequencing, and high throughput sequencing.

High throughput sequencing involves sequencing-by-synthesis, sequencing-by-ligation, and ultra-deep sequencing (such as described in Marguiles et al., Nature 437 (7057): 376-80 (2005)). Sequence-by-synthesis involves synthesizing a complementary strand of the target nucleic acid by incorporating labeled nucleotide or nucleotide analog in a polymerase amplification. Immediately after or upon successful incorporation of a label nucleotide, a signal of the label is measured and the identity of the nucleotide is recorded. The detectable label on the incorporated nucleotide is removed before the incorporation, detection and identification steps are repeated. Examples of sequence-by-synthesis methods are known in the art, and are described for example in U.S. Pat. Nos. 7,056,676, 8,802,368 and 7,169,560, the contents of which are incorporated herein by reference. Sequencing-by-synthesis may be performed on a solid surface (or a microarray or a chip) using fold-back PCR and anchored primers. Target nucleic acid fragments can be attached to the solid surface by hybridizing to the anchored primers, and bridge amplified. This technology is used, for example, in the Illumina© sequencing platform.

Pyrosequencing involves hybridizing the target nucleic acid regions to a primer and extending the new strand by sequentially incorporating deoxynucleotide triphosphates corresponding to the bases A, C, G, and T (U) in the presence of a polymerase. Each base incorporation is accompanied by release of pyrophosphate, converted to ATP by sulfurylase, which drives synthesis of oxyluciferin and the release of visible light. Since pyrophosphate release is equimolar with the number of incorporated bases, the light given off is proportional to the number of nucleotides adding in any one step. The process is repeated until the entire sequence is determined.

In certain embodiments, the detection of mutation and/or wild-type status and the measurement of level of biomarkers of interest described herein is by whole transcriptome sequencing, or RNA sequencing (e.g. RNA-Seq). The method of RNA sequencing has been described (see Wang Z, Gerstein M and Snyder M, Nature Review Genetics (2009) 10:57-63; Maher C A et al., Nature (2009) 458:97-101; Kukurba K & Montgomery S B, Cold Spring Harbor Protocols (2015) 2015(11): 951-969). In brief, mRNA extracted from a sample is reverse transcribed into cDNA and sheared into fragments. Fragments within proper length ranges are selected and ligated with sequencing adaptors, followed by amplification, sequencing, and mapping reads to a reference genome.

In certain embodiments, the CNV of a biomarker is determined using whole exome sequencing (WES). WES involves sequencing DNA exons (i.e. protein encoding regions) using high-throughput sequencing technology. More details of WES can be found, for example, in Ng S B et al, Nature. 461 (7261): 272-276 (2009), and Bao R et al, Cancer Inform. 2014; 13(Suppl 2): 67-82, which are incorporated herein to their entirety.

Immunoassays

Immunoassays typically involves using antibodies that specifically bind to the biomarker polypeptide or protein (e.g. the ATM, ATR, MDM2, and/or p53 protein as provided herein) to detect or measure the presence or level of the target polypeptide or protein. Such antibodies can be obtained using methods known in the art (see, e.g., Huse et al., Science (1989) 246:1275-1281; Ward et al, Nature (1989) 341:544-546), or can be obtained from commercial sources. Examples of immunoassays include, without limitation, Western blotting, enzyme-linked immunosorbent assay (ELISA), enzyme immunoassay (EIA), radioimmunoassay (RIA), sandwich assays, competitive assays, immunofluorescent staining and imaging, immunohistochemistry (IHC), and fluorescent activating cell sorting (FACS). For a review of immunological and immunoassay procedures, see Basic and Clinical Immunology (Stites & Terr eds., 7^{th} ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Terr, eds., 7^{th} ed. 1991).

In certain embodiments, the antibodies are detectably labeled, or alternatively are not labeled but can react with a second molecule which is detectably labeled (e.g. a detectably labeled secondary antibody). Other detection systems such as time-resolved fluorescence, internal-reflection fluorescence, amplification (e.g., polymerase chain reaction) and Raman spectroscopy are also useful.

In certain embodiments, the antibodies may be immobilized on a solid substrate. The immobilization can be via covalent linking or non-covalent attachment (e.g. coating). Examples of solid substrate include porous and non-porous materials, latex particles, magnetic particles, microparticles, strips, beads, membranes, microtiter wells and plastic tubes. The choice of solid phase material and method of detectably labeling the antigen or antibody reagent are determined based upon desired assay format performance characteristics.

Activity Assays

The biological activity of a protein can be measured using a bioassay. For example, ATM and ATR are both DNA damage repair proteins, and their DNA repair activities can be determined by a DNA damage marker in the presence of an inducer for DNA damage; MDM2 is a kinase, and its kinase activity can be measured by detecting the level of MDM2 or phosphorylated MDM2; the activity of p53 can be measured by detecting the phosphorylation of the amino acid residue at position 15 of p53, or by detecting the change in expression level of the downstream target genes of p53. Due to a protein's ability to exert multiple biological activities, several acceptable bioassays may exist for a particular protein. Exemplary functional assays for measuring the activity of ATM, ATR, MDM2 or p53 can be found in Lee J-H et al, J Biol Chem, 288:12840-12851 (2013), Loughery J, et al, Nucleic Acids Research, 42:7666-7680 (2014), Thompson T, et al, Journal Biological Chemistry, 279:53015-53022 (2004), Wienken, M. et al., J. Mol. Cell Biol. 2017; 9(1): 74-80.

iii. Prediction of Responsiveness to Treatment with MDM2 Inhibitor

In certain embodiments, the method further comprises identifying the subject as likely to respond to the treatment of with an MDM2 inhibitor based on: i) the deficiency in activity or level of ATM and/or ATR, or ii) the gain in activity or level of in MDM2, or both i) and ii), found in the biological sample.

In certain embodiments, presence of one or more inactivating mutation in ATM and/or ATR in the biological sample indicates deficiency in activity or level of ATM and/or ATR. In certain embodiments, the subject is identified as likely to respond to the treatment of MDM2 inhibitor based on having one or more inactivating mutation in ATM and/or ATR.

Exemplary inactivating mutations in ATM includes, without limitation, a mutation selected from the group of mutations relative to SEQ ID NO: 2 as listed in FIGS. 1B, 1C and 1D (e.g., H1380Y, N1983S, N2875S, R2598Q, 1599_1600del, V2716A, K1903fs, V2906I, A1127V, K1101E, Q912*, S2165F, or H1083Y), or c.3154-2 A>G relative to SEQ ID NO:1, or any combination thereof. Exemplary inactivating mutations in ATR includes, K243T, Q1926H, I774fs, K1379N, L1483F, or any combination thereof, relative to SEQ ID NO: 4.

In certain embodiments, the subject is identified as likely to respond to the treatment of MDM2 inhibitor based on having one or more inactivating mutations in ATM comprising a mutation selected from the group of mutations relative to SEQ ID NO: 2 as listed in FIGS. 1B, 1C and 1D (e.g., H1380Y, N1983S, N2875S, R2598Q, 1599_1600del, V2716A, K1903fs, V2906I, A1127V, K1101E, Q912*, S2165F, H1083Y), c.3154-2 A>G relative to SEQ ID NO: 1, or any combination thereof, and/or having one or more inactivating mutations in ATR selected from the group consisting of K243T, Q1926H, I774fs, K1379N, L1483F, and any combination thereof, relative to SEQ ID NO: 4, in the biological sample.

In certain embodiments, a decrease (e.g. at least 5%, 10%, 15%, 20%, 25%, 30%, 35%, 40%, 45%, 50%, 55%, 60%, 65%, 70%, 75%, 80%, 85%, 90%, or 95% decrease) in expression level of ATM and/or ATR gene product relative to a reference level of ATM and/or ATR gene product respectively, indicates deficiency in activity or level of ATM and/or ATR in the biological sample.

In certain embodiments, an elevation in copy number variation (CNV) of MDM in the biological sample, relative to a reference level of CNV of MDM, indicates gain in activity or level of MDM2.

In certain embodiments, a subject having a copy number variation (CNV) of >3 in MDM2 gene is considered having a gain in MDM2 gene. CNV>3 means that the copy number of the gene in the genome of the subject is found to be above 3.

In certain embodiments, an increase (e.g. by at least 50% (e.g. at least 60%, 70%, 80%, 90%, 100%, 110%, 120%, 130%, 140%, or 150%) in expression level of MDM2 gene product, relative to a reference level of MDM2 gene product, indicates gain in activity or level of MDM2.

In certain embodiments, the subject is identified as likely to respond to the treatment of MDM2 inhibitor based on having an elevation in copy number variation (CNV) of MDM in the biological sample, relative to a reference level of CNV of MDM; and/or having an increase in expression level of MDM2 gene product in the biological sample, relative to a reference level of MDM2 gene product.

In certain embodiments, the subject is identified as likely to respond to the treatment of MDM2 inhibitor based on: both i) having one or more inactivating mutation in ATM and/or ATR, and ii) having an elevation in copy number variation (CNV) of MDM in the biological sample, relative to a reference level of CNV of MDM2 in the biological sample. In certain embodiments, the subject is identified as likely to respond to the treatment of MDM2 inhibitor based on: both i) having one or more inactivating mutation in ATM and/or ATR, and ii) having an increase in expression level of MDM2 gene product, relative to a reference level of MDM2 gene product, in the biological sample.

In certain embodiments, the method further comprises determining in the biological sample presence or absence of a functional p53. In certain embodiments, the method further comprises determining in the biological sample if p53 is wild-type. In certain embodiments, the method further comprises identifying the subject as likely to respond to the treatment with an MDM2 inhibitor based on: i) the presence of one or more inactivating mutation in ATM and/or ATR; ii) elevation in the CNV of the MDM2 gene or the increase in the expression level of the MDM2 gene product; and iii) presence of functional p53 (e.g. wild-type p53), found in the biological sample.

In certain embodiments, the expression level of the biomarker can be normalized to an internal control value or to a standard curve. For example, the level of each of the biomarkers described herein can be normalized to a standard level for a standard marker. The standard level of the standard marker can be predetermined, determined concurrently, or determined after a sample is obtained from the subject. The standard marker can be run in the same assay or can be a known standard marker from a previous assay. In the cases when the level of the biomarker is determined by sequencing assay (such as RNA sequencing), the level of the biomarkers can be normalized to the total reads of the sequencing.

The term “reference level” of a biomarker described herein can be the normal or baseline level of the biomarker, for example, a level of the biomarker in the healthy cell or tissue sample, or an average level of the biomarker in a general cancer patient population or in a cancer patient population of a particular cancer of interest.

In certain embodiments, the reference level can be a typical level, a measured level, or a range of the level of the corresponding biomarker that would normally be observed in one or more healthy cell or tissue samples, or in one or more control cell or tissue samples. In certain embodiments, the reference level can be an average level of the corresponding biomarker in a healthy subject population, or in a general cancer patient population or in a cancer patient population of a particular cancer of interest. For example, it can be an empirical level of the biomarker that is considered to be representative of a control sample or a general cancer sample. In certain embodiments, the reference level of the biomarkers described herein is obtained using the same or comparable measurement method or assay as used in the measurement of the level of the biomarker provided herein.

A “general cancer patient population” as used herein, refers to a population of cancer subjects or patients having different kinds of cancers. For example, a general cancer patient population may be a group of at least three (four, five, six, seven, eight, nine, ten, or more) types of cancer patients, with some patients having the first type of cancer, some having the second type of cancer, some having the third type of cancer, and so on. For example, a general cancer patient population can be a population having all kinds of cancers or a variety of cancer types. In certain embodiments, the reference level can also be an empirical level considered representative of a general cancer patient population.

In certain embodiments, the reference level can be predetermined. For example, the reference level can be calculated or generalized based on measurements of the biomarker level in a collection of control biological samples (e.g. samples from healthy subjects, or samples from control cancer patients). For another example, the reference level can be based on statistics of the level of the biomarkers generally observed in healthy subjects, or in general cancer patient population.

IV. Treating the Subject Identified as Likely to Respond to the Treatment with MDM2 Inhibitor

In certain embodiments, the methods provided herein further comprises administering the MDM2 inhibitor to the subject identified as likely to respond to the treatment with an MDM2 inhibitor. In certain embodiments, the MDM2 inhibitor is administered at a therapeutically effective amount to the subject.

In certain embodiments, the present disclosure provides methods of treating a subject with cancer with an MDM2 inhibitor, wherein the subject has been identified as likely to respond to the treatment with the MDM2 inhibitor by any of the methods provided herein. In certain embodiments, the step of treating comprising administering a therapeutically effective amount of the MDM2 inhibitor to the subject having been identified as likely to respond to the treatment with the MDM2 inhibitor.

The MDM2 inhibitors disclosed in the present invention inhibit the interaction between p53 or p53-related proteins and MDM2 or MDM2-related proteins. By inhibiting the negative effect of MDM2 or MDM2-related proteins on p53 or p53-related proteins, the MDM2 inhibitors of the present invention sensitize cells to inducers of apoptosis and/or cell cycle arrest. In one embodiment, the MDM2 inhibitors of the present invention induce apoptosis and/or cell cycle arrest.

Activities of MDM2 inhibitors can be determined by fluorescence-polarization MDM2 binding assay, a competitive binding assay between MDM2 inhibitors and a p53-based peptidomimetic compound competing for binding to a MDM2 protein as described in U.S. Pat. No. 9,745,314B2.

Fluorescence polarization measurement of competitive binding works by titrating a mixture of a protein of interest and a fluorescently labeled probe with an unlabeled competitor and demonstrating that the fluorescence polarization decreases to the value observed with the free fluorescently labeled probe (Moerke N, Current Protocols in Chemical Biology, (2009)1:1). In the fluorescence-polarization MDM2 binding assay, a recombinant human His-tagged MDM1 protein (residue 1-118) and a fluorescently tagged p53-based peptide called PMDM6-F (Garcia-Echeverria et al., J. Med. Chem. 43: 3205-3208 (2000)) are used, and the Kd value of PMDM6-F with the recombinant MDM2 protein is determined. A dose-dependent, competitive binding experiments are then performed with serial dilutions of a tested MDM2 inhibitor in the presence of pre-incubated MDM2 protein and PMDM6-F peptide. The polarization values are measured and the IC50 values are determined from a plot using nonlinear least-squares analysis.

In some embodiments, the MDM2 inhibitors has an IC50 of no more than 1 μM, e.g. no more than 900 nM, 800 nM, 700 nM, 600 nM, 500 nM, 400 nM, 300 nM, 200 nM, 150 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM or 1 nM, in inhibiting the binding of MDM2 to P53 as determined by the fluorescence-polarization MDM2 binding assay.

In some embodiments, MDM2 inhibitor is selected from idasanutlin (RG7388), RG7112 (PubChem Compound CID: 57406853), HDM201 (PubChem Compound CID: 71678098), KRT-232 (also known as AMG232, PubChem Compound CID: 58573469), AMG 232 (PubChem Compound CID: 58573469), BI907828 (accessible in NCI Thesaurus (version: 19.10d) under code C156709), SAR-405838 (also known as MI-77301, PubChem Compound CID: 53476877), MK-8242 (also known as SCH 900242, accessible in NCI Thesaurus (version: 19.10d) under code C116867), DS3032-b (PubChem Compound CID: 9051550), ALRN-6924 (PubChem Compound CID: 381833444) and CGM097 ((PubChem Compound CID: 53240420); or a pharmaceutically acceptable salt of any of the foregoing.

In some embodiments, the MDM2 inhibitor comprises a compound represented by formula (I):

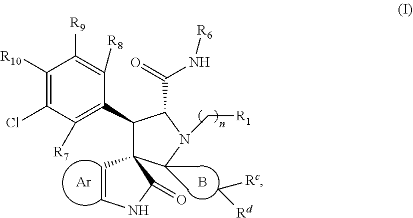

or a pharmaceutically acceptable salt thereof, wherein

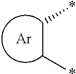

is selected from the group consisting of

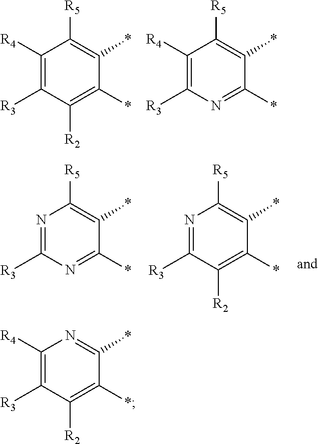

B is a C₄₋₇ carbocyclic ring;

R₁ is H, substituted or unsubstituted C₁₋₄ alkyl, substituted or unsubstituted cycloalkyl, substituted or unsubstituted heterocycloalkyl, OR^{a}, or NR^{a}R^{b};

n is 0, 1, or 2;

R₂, R₃, R₄, R₅, R₇, R₈, R₉, and R₁₀, independently, are selected from the group consisting of H, F, Cl, CH₃, and CF₃;

R⁶ is

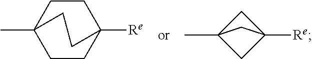

R^{a} is hydrogen or substituted or unsubstituted C₁₋₄ alkyl;

R^{b} is hydrogen or substituted or unsubstituted C₁₋₄ alkyl;

R^{c} and R^{d} are substituents on one carbon atom of ring B, wherein

R^{c} is H, C₁₋₃ alkyl, C₁₋₃ alkylene-OR^{a}, OR^{a}, or halo;

R^{d} is H, C₁₋₃ alkyl, C₁₋₃ alkylene-OR^{a}, OR^{a}, or halo; or

R^{c} and R^{d} are taken together with the carbon to which they are attached to form a 4 to 6-membered Spiro substituent, optionally containing an oxygen atom; and

R^{e} is C(═O)OR^{a}, C(═O)NR^{a}R^{b}, or C(═O)NHSO₂CH₃.

As used herein, the term “alkyl” refers to straight chained and branched saturated C₁₋₁₀ hydrocarbon groups, including but not limited to methyl, ethyl, n-propyl, i-propyl, n-butyl, sec-butyl, t-butyl, n-pentyl, 2-methylbutyl, 3-methylbutyl, 2,2-dimethylpropyl, n-hexyl, 2-methylpentyl, 3-methylpentyl, 4-methylpentyl, 2,2-dimethylbutyl, 2,3-dimethylbutyl, 3,3-dimethylbutyl, and 2-ethylbutyl. The term Cₘ₋ₙ means the alkyl group has “m” to “n” carbon atoms. The term “alkylene” refers to an alkyl group having a substituent. An alkyl, e.g., methyl, or alkylene, e.g., —CH₂—, group can be substituted with one or more, and typically one to three, of independently selected halo, trifluoromethyl, trifluoromethoxy, hydroxy, alkoxy, nitro, cyano, alkylamino, or amino groups, for example.

As used herein, the term “halo” is defined as fluoro, chloro, bromo, or iodo.

The term “hydroxy” is defined as —OH.

The term “alkoxy” is defined as —OR, wherein R is alkyl.

The term “amino” is defined as —NH₂, and the term “alkylamino” is defined as —NR₂, wherein at least one R is alkyl and the second R is alkyl or hydrogen.

The term “carbamoyl” is defined as —C(═O)NR₂.

The term “carboxy” is defined as —C(═O)OH or a salt thereof.

The term “nitro” is defined as —NO₂.

The term “cyano” is defined as —CN.

The term “trifluoromethyl” is defined as —CF₃.

The term “trifluoromethoxy” is defined as —OCF₃.

As used herein, the term “aryl” refers to a monocyclic or polycyclic aromatic group, preferably a monocyclic or bicyclic aromatic group. Examples of aryl groups include, but are not limited to, phenyl, naphthyl, fluorenyl, azulenyl, anthryl, phenanthryl, pyrenyl, biphenyl, and terphenyl. Aryl also refers to bicyclic and tricyclic carbon rings, where one ring is aromatic and the others are saturated, partially unsaturated, or aromatic, for example, dihydronaphthyl, indenyl, indanyl, or tetrahydronaphthyl (tetralinyl). Unless otherwise indicated, an aryl group can be unsubstituted or substituted with one or more, and in particular one to four, groups independently selected from, for example, halo, alkyl, alkenyl, —OCF₃, —NO₂, —CN, —NC, —OH, alkoxy, amino, alkylamino, —CO₂H, —CO₂alkyl, —OCOalkyl, aryl, and heteroaryl.

As used herein, the term “heterocyclic” refers to a heteroaryl and heterocycloalkyl ring systems.

As used herein, the term “heteroaryl” refers to a monocyclic or bicyclic ring system containing one or two aromatic rings and containing at least one nitrogen, oxygen, or sulfur atom in an aromatic ring. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl group has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzothiophenyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quiazolinyl, thiadiazolopyrimidyl, and thienopyridyl. Unless otherwise indicated, a heteroaryl group can be unsubstituted or substituted with one or more, and in particular one to four, substituents selected from, for example, halo, alkyl, alkenyl, —OCF₃, —NO₂, —CN, —NC, —OH, alkoxy, amino, alkylamino, —CO₂H, —CO₂alkyl, —OCOalkyl, aryl, and heteroaryl.

As used herein, the term “cycloalkyl” means a monocyclic or bicyclic, saturated or partially unsaturated, ring system containing three to eight carbon atoms, including cyclopropyl, cyclobutyl, cyclopentyl, cyclohexyl, cycloheptyl, and cyclooctyl, optionally substituted with one or more, and typically one to three, of independently selected halo, trifluoromethyl, trifluoromethoxy, hydroxy, alkoxy, nitro, cyano, alkylamino, or amino groups, for example.

As used herein, the term “heterocycloalkyl” means a monocyclic or a bicyclic, saturated or partially unsaturated, ring system containing 4 to 12 total atoms, of which one to five of the atoms are independently selected from nitrogen, oxygen, and sulfur and the remaining atoms are carbon. Non-limiting examples of heterocycloalkyl groups are azetidinyl, pyrrolidinyl, piperidinyl, piperazinyl, dihydropyrrolyl, morpholinyl, thiomorpholinyl, dihydropyridinyl, oxacycloheptyl, dioxacycloheptyl, thiacycloheptyl, diazacycloheptyl, each optionally substituted with one or more, and typically one to three, of independently selected halo, C₁₋₆ alkyl, C₁₋₆ alkoxy, cyano, amino, carbamoyl, nitro, carboxy, C₂₋₇ alkenyl, C₂₋₇ alkynyl, or the like on an atom of the ring.

In some embodiments,

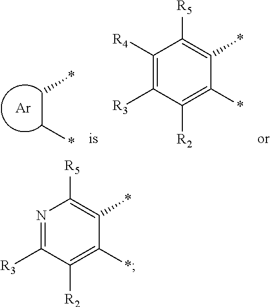

In some embodiments, B is

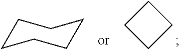

In some embodiments, n is 0 or 1 and R₁ is H or CH₃.

In some embodiments,

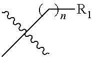

is H, CH₃, or CH₂CH₃.

In some embodiments, R₂ is H. In other embodiments, R₃ is halo, and preferably chloro. In still another embodiments, R₄ is H, R₅ is H, or both R₄ and R₅ are H.

In some embodiments, R₇ is halo, and more preferably is fluoro.

In some embodiments, each of R⁸, R⁹, and R¹⁰ are H.

In some embodiments, R^{a} and R^{b}, individually, are H, CH₃, or CH₂CH₃.

In some embodiments, R^{c} and R^{d}, individually, are H, halo, OH, CH₃, CH₂CH₃, or CH₂OH.

In some embodiments, R^{c} and R^{d} are respectively F and F, H and H, OH and CH₃, OH and H, CH₃ and CH₃, CH₃ and OH, H and OH, CH₂CH₃ and CH₂CH₃, and CH₂OH and CH₂OH.

In some embodiments, R^{e} is —C(—O)OH, —C(—O)NH₂, or —C(—O)NHSO₂CH₃.

In one embodiment, the MDM2 inhibitor is a compound selected from

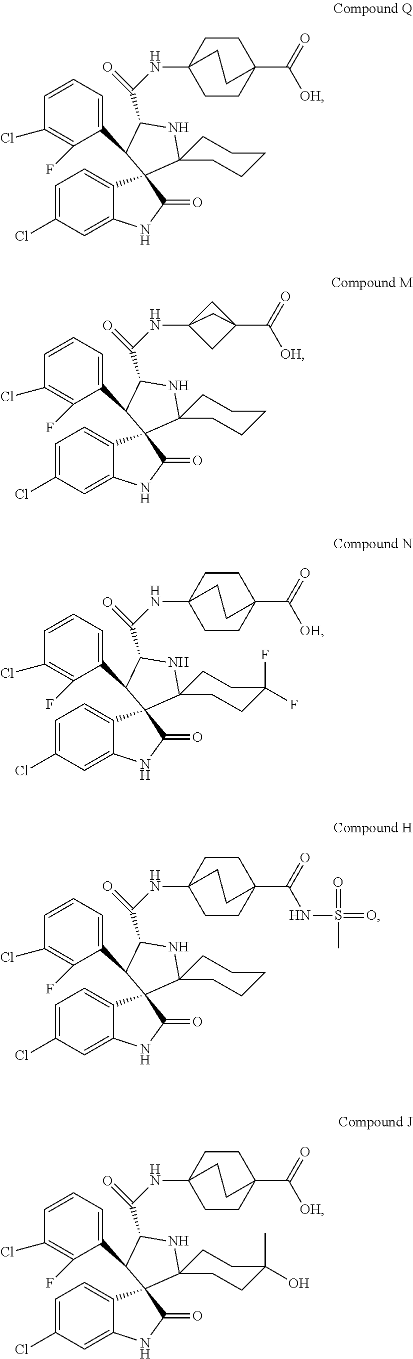 [SMILES: O=C(NC12CCC(C(=O)O)(CC1)CC2)[C@@H]1NC2(CCCCC2)[C@@]2(C(=O)Nc3cc(Cl)ccc32)[C@H]1c1cccc(Cl)c1F ; O=C(NC12CC(C(=O)O)(C1)C2)[C@@H]1NC2(CCCCC2)[C@@]2(C(=O)Nc3cc(Cl)ccc32)[C@H]1c1cccc(Cl)c1F ; O=C(NC12CCC(C(=O)O)(CC1)CC2)[C@@H]1NC2(CCC(F)(F)CC2)[C@@]2(C(=O)Nc3cc(Cl)ccc32)[C@H]1c1cccc(Cl)c1F ; CS(=O)(=O)NC(=O)C12CCC(NC(=O)[C@@H]3NC4(CCCCC4)[C@@]4(C(=O)Nc5cc(Cl)ccc54)[C@H]3c3cccc(Cl)c3F)(CC1)CC2 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21]

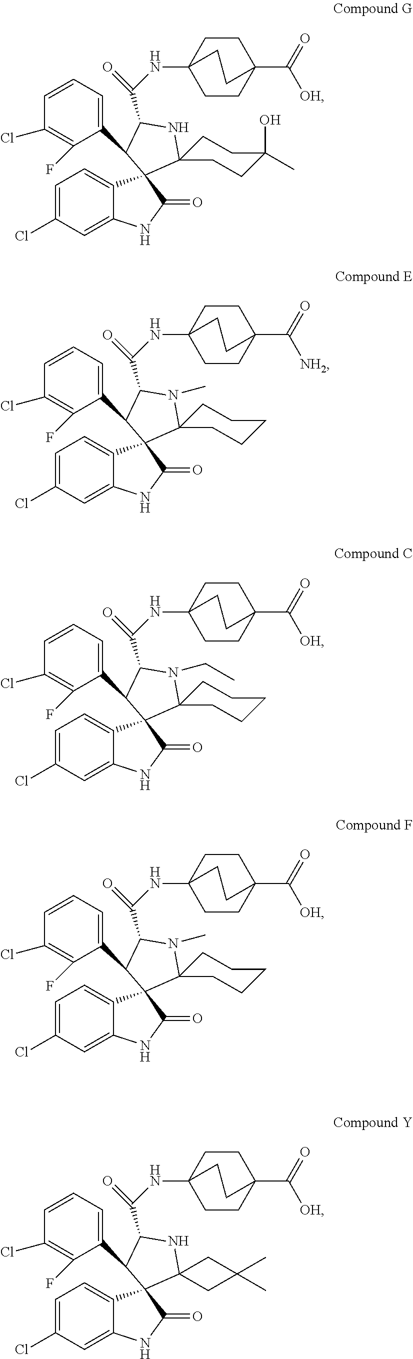 [SMILES: CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21 ; CN1[C@@H](C(=O)NC23CCC(C(N)=O)(CC2)CC3)[C@H](c2cccc(Cl)c2F)[C@]2(C(=O)Nc3cc(Cl)ccc32)C12CCCCC2 ; CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CC1(C)CC2(C1)N[C@@H](C(=O)NC13CCC(C(=O)O)(CC1)CC3)[C@H](c1cccc(Cl)c1F)[C@]21C(=O)Nc2cc(Cl)ccc21]

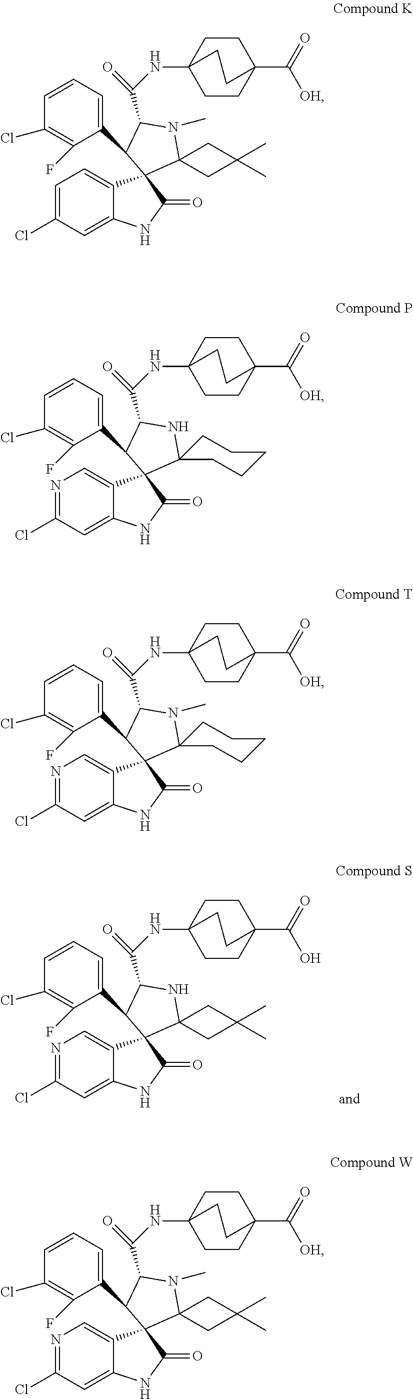 [SMILES: CN1[C@@H](C(=O)NC23CCC(C(=O)O)(CC2)CC3)[C@H](c2cccc(Cl)c2F)[C@]2(C(=O)Nc3cc(Cl)ccc32)C12CC(C)(C)C2 ; O=C(NC12CCC(C(=O)O)(CC1)CC2)[C@@H]1NC2(CCCCC2)[C@@]2(C(=O)Nc3cc(Cl)ncc32)[C@H]1c1cccc(Cl)c1F ; CN1[C@@H](C(=O)NC23CCC(C(=O)O)(CC2)CC3)[C@H](c2cccc(Cl)c2F)[C@]2(C(=O)Nc3cc(Cl)ncc32)C12CCCCC2 ; CC1(C)CC2(C1)N[C@@H](C(=O)NC13CCC(C(=O)O)(CC1)CC3)[C@H](c1cccc(Cl)c1F)[C@]21C(=O)Nc2cc(Cl)ncc21 ; CN1[C@@H](C(=O)NC23CCC(C(=O)O)(CC2)CC3)[C@H](c2cccc(Cl)c2F)[C@]2(C(=O)Nc3cc(Cl)ncc32)C12CC(C)(C)C2]

or a pharmaceutically acceptable salt of the compound.

In one embodiment, the MDM2 inhibitor is

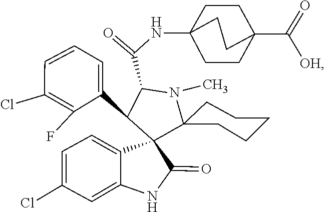 [SMILES: CN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

or a pharmaceutically acceptable salt thereof.

In one embodiment, the MDM2 inhibitor is

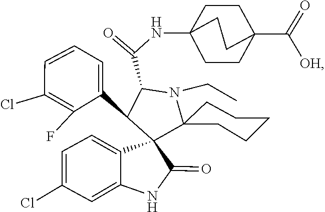 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

or a pharmaceutically acceptable salt thereof.

More MDM2 inhibitors and the synthesis of the MDM2 inhibitors that can be used in the present application are further disclosed in U.S. Pat. No. 9,745,314, which is incorporated herein by reference.

The MDM2 inhibitors provided herein can exist as salts. Pharmaceutically acceptable salts of the MDM2 inhibitors provided herein often are preferred in the methods of the invention. As used herein, the term “pharmaceutically acceptable salts” refers to salts or zwitterionic forms of the compounds of structural formula (I). Salts of compounds of formula (I) can be prepared during the final isolation and purification of the compounds or separately by reacting the compound with an acid having a suitable cation, such as, but not limited to, alkali and alkaline earth metal ions, e.g., Na⁺, K⁺, Ca²⁺, and Mg²⁺ well as organic cations such as, but not limited to, ammonium and substituted ammonium ions, e.g., NH₄⁺, NHMe₃⁺, NH₂Me₂⁺, NHMe₃⁺ and NMe₄⁺. Examples of monovalent and divalent pharmaceutically acceptable cations are discussed, e.g., in Berge et al. J. Pharm. Sci., 66:1-19 (1997).

The pharmaceutically acceptable salts of compounds of structural formula (I) can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the invention include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulphonate, and p-toluenesulfonate salts. In addition, available amino groups present in the compounds of the invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to compounds of the present invention appearing herein is intended to include compounds of structural formula (I) as well as pharmaceutically acceptable salts thereof.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof.

The term “geometric isomer” refers to cyclic compounds having at least two substituents, wherein the two substituents are both on the same side of the ring (cis) or wherein the substituents are each on opposite sides of the ring (trans). When a disclosed compound is named or depicted by structure without indicating stereochemistry, it is understood that the name or the structure encompasses one or more of the possible stereoisomers, or geometric isomers, or a mixture of the encompassed stereoisomers or geometric isomers.

When a geometric isomer is depicted by name or structure, it is to be understood that the named or depicted isomer exists to a greater degree than another isomer, that is that the geometric isomeric purity of the named or depicted geometric isomer is greater than 50%, such as at least 60%, 70%, 80%, 90%, 99%, or 99.9% pure by weight. Geometric isomeric purity is determined by dividing the weight of the named or depicted geometric isomer in the mixture by the total weight of all of the geometric isomers in the mixture.

Racemic mixture means 50% of one enantiomer and 50% of is corresponding enantiomer. When a compound with one chiral center is named or depicted without indicating the stereochemistry of the chiral center, it is understood that the name or structure encompasses both possible enantiomeric forms (e.g., both enantiomerically-pure, enantiomerically-enriched or racemic) of the compound. When a compound with two or more chiral centers is named or depicted without indicating the stereochemistry of the chiral centers, it is understood that the name or structure encompasses all possible diasteriomeric forms (e.g., diastereomerically pure, diastereomerically enriched and equimolar mixtures of one or more diastereomers (e.g., racemic mixtures)) of the compound.

Enantiomeric and diastereomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and diastereomers also can be obtained from diastereomerically- or enantiomerically-pure intermediates, reagents, and catalysts by well-known asymmetric synthetic methods.

When a compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers is included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

In certain embodiments, the MDM2 inhibitor is administered as a pharmaceutical composition. The pharmaceutical composition can comprise an MDM2 inhibitor or a pharmaceutically acceptable salt thereof, and a pharmaceutically acceptable carrier or diluent.

“Pharmaceutically acceptable carrier” and “pharmaceutically acceptable diluent” refer to a substance that aids the formulation and/or administration of an active agent to and/or absorption by a subject and can be included in the compositions of the present disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, hydroxymethycellulose, fatty acid esters, polyvinyl pyrrolidine, and colors, and the like. The carriers, diluents and/or excipients are “acceptable” in the sense of being compatible with the other ingredients of the pharmaceutical composition and not deleterious to the recipient thereof.

In some embodiments, the pharmaceutical composition comprises an MDM2 inhibitor of the following structure known as Compound C

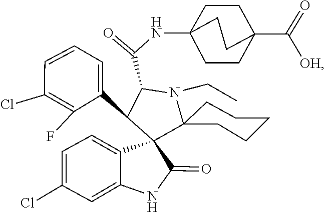 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

or a pharmaceutically acceptable salt thereof. In some embodiments, the pharmaceutical composition is in a solid dose form. In some embodiments, the solid dose form is capsules. In some embodiments, the solid dose form is dry-filled capsules. In some embodiments, the solid dose form is dry-filled size 1 gelatin capsules. In some embodiments, the capsule comprises from about 10-500 mg of an MDM2 inhibitor, such as Compound C. In some embodiments, the pharmaceutical composition or capsule comprises silicified microcrystalline cellulose.

In some embodiments, the pharmaceutical composition or capsules comprises an MDM2, such as Compound C, in an amount of about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 120 mg, 130 mg, 140 mg, 150 mg, 160 mg, 170 mg, 180 mg, 190 mg, 210 mg, 220 mg, 230 mg, 240 mg, 250 mg, 260 mg, 270 mg, 280 mg, 290 mg, or 300 mg.

In certain embodiments, the MDM2 inhibitor (e.g., Compound C) is administered in at least one dose per day. In certain embodiments, the MDM2 inhibitor (e.g., Compound C) is administered in at least two doses per day. In certain embodiments, the MDM2 inhibitor (e.g., Compound C) is administered in at least three dose per day. In certain embodiments, the MDM2 inhibitor (e.g., Compound C) is administered in one dose per day. In certain embodiments, the MDM2 inhibitor (e.g., Compound C) is administered in two doses per day. In certain embodiments, the MDM2 inhibitor (e.g., Compound C) is administered in three doses per day. Additional suitable treatment regimens for MDM2 inhibitor (e.g., Compound C) are provided, for example, in U.S. Pat. No. 9,745,314, the entire contents of which are expressly incorporated herein by reference.

One skilled in the art would be able, by routine experimentation, to determine what an effective, non-toxic amount of an MDM2 inhibitor (e.g., Compound C) would be for the purpose of treating cancers. For example, a therapeutically active amount of MDM2 inhibitor (e.g., Compound C) may vary according to factors such as the disease stage (e.g., stage I versus stage IV), age, sex, medical complications (e.g., immunosuppressed conditions or diseases) and weight of the subject, and the ability of the MDM2 inhibitor (e.g., Compound C) to elicit a desired response in the subject. In certain embodiments, a therapeutically active amount is a safe amount of MDM2 inhibitor which is sufficient to yield a desired therapeutic response without undue adverse side effects (such as toxicity, irritation, or allergic response) commensurate with a reasonable benefit/risk ratio when used in the manner of this disclosure.

The dosage regimen may be adjusted to provide the optimum therapeutic response. For example, several divided doses may be administered daily or administered by continuous infusion or the dose may be proportionally reduced as indicated by the exigencies of the therapeutic situation. In certain embodiments, an MDM2 inhibitor (e.g., Compound C) is administered in an amount that would be therapeutically effective if delivered alone, i.e., MDM2 inhibitor (e.g., Compound C) is administered and/or acts as a therapeutic anti-cancer agent, and not predominantly as an agent to ameliorate side effects of other chemotherapy or other cancer treatments.

In certain embodiments, an MDM2 inhibitor (e.g., Compound C) is administered in an amount that would be effective to improve or augment the immune response to the tumor. The dosages provided below may be used for any mode of administration of MDM2 inhibitor (e.g., Compound C), including topical administration, administration by inhalation, and intravenous administration (e.g. continuous infusion).

In certain embodiments, the MDM2 inhibitor (e.g., Compound C) is administered in the range of about 0.5 mg/kg to about 10,000 mg/kg, about 5 mg/kg to about 5,000 mg/kg, about 10 mg/kg to about 3,000 mg/kg. In one embodiment, an MDM2 inhibitor (e.g., Compound C) is administered in the range of about 10 mg/kg to about 1,400 mg/kg. In one embodiment, MDM2 inhibitor (e.g., Compound C) is administered in the range of about 10 mg/kg to about 650 mg/kg. In one embodiment, an MDM2 inhibitor (e.g., Compound C) is administered in the range of about 10 mg/kg to about 200 mg/kg. In various embodiments, an MDM2 inhibitor (e.g., Compound C) is administered at a dose of about 1 mg/kg, 2 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 120 mg/kg, 130 mg/kg, 140 mg/kg, 150 mg/kg, 160 mg/kg, 170 mg/kg, 180 mg/kg, 190 mg/kg, 210 mg/kg, 220 mg/kg, 230 mg/kg, 240 mg/kg, 250 mg/kg, 260 mg/kg, 270 mg/kg, 280 mg/kg, 290 mg/kg, or 300 mg/kg.

It should be understood that ranges having any one of these values as the upper or lower limits are also intended to be part of this invention, e.g., about 50 mg/kg to about 200 mg/kg, or about 650 mg/kg to about 1400 mg/kg. In one embodiment the administered dose is at least about 1 mg/kg, 2 mg/kg, at least about 5 mg/kg, at least about 10 mg/kg, at least about 12.5 mg/kg, at least about 20 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, at least about 100 mg/kg, at least about 104 mg/kg, at least about 125 mg/kg, at least about 150 mg/kg, at least about 175 mg/kg, at least about 200 mg/kg, at least about 250 mg/kg, at least about 300 mg/kg, or at least about 400 mg/kg.

In certain embodiments, the MDM2 inhibitor (e.g., Compound C) is administered at a dose of about 10 mg/kg/day (24 hours) to about 150 mg/kg/day (24 hours). In certain embodiments, the MDM2 inhibitor (e.g., Compound C) is administered at a dose selected from the group consisting of about 1 mg/kg/day (24 hours), about 2 mg/kg/day (24 hours), about 5 mg/kg/day (24 hours), about 10 mg/kg (24 hours), about 15 mg/kg/day (24 hours), about 20 mg/kg/day (24 hours), about 25 mg/kg/day (24 hours), about 30 mg/kg/day (24 hours), about 35 mg/kg/day (24 hours), about 40 mg/kg/day (24 hours), about 45 mg/kg/day (24 hours), about 50 mg/kg/day (24 hours), about 55 mg/kg/day (24 hours), about 60 mg/kg/day (24 hours), about 65 mg/kg/day (24 hours), 70 mg/kg/day (24 hours), about 75 mg/kg/day (24 hours), about 80 mg/kg/day (24 hours), about 85 mg/kg/day (24 hours), about 90 mg/kg/day (24 hours), about 95 mg/kg/day (24 hours), about 100 mg/kg/day (24 hours), about 105 mg/kg/day (24 hours), about 110 mg/kg/day (24 hours), about 120 mg/kg/day (24 hours), about 130 mg/kg/day (24 hours), about 140 mg/kg/day (24 hours), about 150 mg/kg/day (24 hours), about 160 mg/kg/day (24 hours), about 170 mg/kg/day (24 hours), about 180 mg/kg/day (24 hours), about 190 mg/kg/day (24 hours), about 200 mg/kg/day (24 hours), about 210 mg/kg/day (24 hours), about 220 mg/kg/day (24 hours), about 230 mg/kg/day (24 hours), about 240 mg/kg/day (24 hours), about 250 mg/kg/day (24 hours), about 260 mg/kg/day (24 hours), about 270 mg/kg/day (24 hours), about 280 mg/kg/day (24 hours), about 290 mg/kg/day (24 hours), and about 300 mg/kg/day (24 hours).

In certain embodiments, the MDM2 inhibitor (e.g., Compound C) is administered at a dose of about 10 mg/day (24 hours) to about 150 mg/day (24 hours). In certain embodiments, the MDM2 inhibitor (e.g., Compound C) is administered at a dose selected from the group consisting of about 1 mg/day (24 hours), about 2 mg/day (24 hours), about 5 mg/day (24 hours), about 10 mg/kg (24 hours), about 15 mg/day (24 hours), about 20 mg/day (24 hours), about 25 mg/day (24 hours), about 30 mg/day (24 hours), about 35 mg/kg/day (24 hours), about 40 mg/day (24 hours), about 45 mg/day (24 hours), about 50 mg/day (24 hours), about 55 mg/day (24 hours), about 60 mg/day (24 hours), about 65 mg/day (24 hours), 70 mg/day (24 hours), about 75 mg/day (24 hours), about 80 mg/day (24 hours), about 85 mg/day (24 hours), about 90 mg/day (24 hours), about 95 mg/day (24 hours), about 100 mg/day (24 hours), about 105 mg/day (24 hours), about 110 mg/day (24 hours), about 120 mg/day (24 hours), about 130 mg/day (24 hours), about 140 mg/day (24 hours), about 150 mg/day (24 hours), about 160 mg/day (24 hours), about 170 mg/day (24 hours), about 180 mg/day (24 hours), about 190 mg/day (24 hours), about 200 mg/day (24 hours), about 210 mg/day (24 hours), about 220 mg/day (24 hours), about 230 mg/day (24 hours), about 240 mg/day (24 hours), about 250 mg/day (24 hours), about 260 mg/day (24 hours), about 270 mg/day (24 hours), about 280 mg/day (24 hours), about 290 mg/day (24 hours), and about 300 mg/day (24 hours).

In certain embodiments, the MDM2 inhibitor (e.g., Compound C) is administered at a dose of about 10 mg/kg/week. In certain embodiments, the MDM2 inhibitor (e.g., Compound C) is administered at a dose of about 25 mg/kg/week. In certain embodiments, the MDM2 inhibitor (e.g., Compound C) is administered at a dose of about 50 mg/kg/week. In certain embodiments, the MDM2 inhibitor (e.g., Compound C) is administered at a dose of about 75 mg/kg/week. In certain embodiments, the MDM2 inhibitor (e.g., Compound C) is administered at a dose of about 100 mg/kg/week. In certain embodiments, the MDM2 inhibitor (e.g., Compound C) is administered at a dose of about 125 mg/kg/week. In certain embodiments, the MDM2 inhibitor (e.g., Compound C) is administered at a dose of about 150 mg/kg/week. In certain embodiments, the MDM2 inhibitor (e.g., Compound C) is administered at a dose selected from the group consisting of about 5 mg/kg/week, about 10 mg/kg/week, about 25 mg/kg/week, about 50 mg/kg/week, about 75 mg/kg/week, about 100 mg/kg/week, about 125 mg/kg/week, about 150 mg/kg/week, about 175 mg/kg/week, about 200 mg/kg/week, about 225 mg/kg/week, about 250 mg/kg/week, about 300 mg/kg/week, about 350 mg/kg/week, about 400 mg/kg week, about 450 mg/kg/week, about 500 mg/kg/week, about 550 mg/kg/week, about 600 mg/kg/week, about 650 mg/kg/week, and about 700 mg/kg/week.

In some embodiments, an MDM2 inhibitor, such as Compound C, or pharmaceutically acceptable salt thereof, is administered orally every other day (QOD). In some embodiments, the MDM2 inhibitor, such as Compound C, or pharmaceutically acceptable salt thereof, is administered orally in an amount from about 30 mg to about 250 mg every other day. In some embodiments, the MDM2 inhibitor, such as Compound C, or pharmaceutically acceptable salt thereof, is administered orally in an amount from about 50 mg to about 200 mg every other day. In some embodiments, the MDM2 inhibitor, such as Compound C, or pharmaceutically acceptable salt thereof, is administered orally in an amount of about 50 mg, 100 mg, 150 mg, or 200 mg every other day.

Notably, when a dose range from 10 mg/kg to 50 mg/kg for an MDM2 inhibitor (e.g., Compound C) was used in mice as disclosed in the examples provided herein, the corresponding clinically relevant doses are 48.8 and 244 mg/day for a 60 kg human, respectively. A factor of 12.3 was used for converting mouse dose to human equivalent dose (HED) here. To convert animal dose in mg/kg to HED mg/m², multiply by km*. See “Guidance for Industry Estimating the Maximum Safe Starting Dose in Initial Clinical Trials for Therapeutics in Adult Healthy Volunteers” U.S. Department of Health and Human Services, Food and Drug Administration, Center for Drug Evaluation and Research (CDER), July 2005, Pharmacology and Toxicology.

Combination Therapy

In some embodiments, the MDM2 inhibitor may also be administered in combination with one or more additional therapies to the subject identified as likely to respond to the treatment with an MDM2 inhibitor by any of the methods provided herein. In some embodiments, the method of treating a subject with cancer comprises administering to the subject with a therapeutically effective amount of an MDM2 inhibitor in combination with one or more additional therapies, wherein the subject has been determined to have i) deficiency in activity or level of ATM and/or ATR, or ii) gain in activity or level of MDM2, or both i) and ii) in a biological sample from the subject.

In some embodiments, the MDM2 inhibitor (e.g., Compound C) is administered at a dosage that is different (e.g. lower) than the standard dosages of the MDM2 inhibitor used to treat the cancer under the standard of care for treatment for a particular cancer. In certain embodiments, the administered dosage of the MDM2 inhibitor (e.g., Compound C) is 5%, 10%, 20%, 30%, 40%, 50%, 60%, 70%, 80%, or 90% lower than the standard dosage of the MDM2 inhibitor (e.g., Compound C) molecule for a particular cancer. In certain embodiments, the dosage administered of the MDM2 inhibitor (e.g., Compound C) molecule is 95%, 90%, 85%, 80%, 75%, 70%, 65%, 60%, 55%, 50%, 45%, 40%, 35%, 30%, 25%, 20%, 15%, 10% or 5% of the standard dosage of the MDM2 inhibitor (e.g., Compound C) molecule for a particular cancer.

In some embodiments, the one or more additional therapy comprises a radiotherapy, a chemotherapy, a targeted cancer therapy, or a therapy with a modulator of an immune checkpoint molecule. In some embodiments, the one or more additional therapy comprise administering an anti-PD-1 antibody, a Bcl-2 inhibitor, a FAK inhibitor, a MEK inhibitor, or a MET inhibitor. It is noted that the additional therapy may comprise administering traditional small organic chemical molecules or macromolecules such as a proteins, antibodies, peptibodies, DNA, RNA or fragments of such macromolecules.

As used herein, the term “radiotherapy” refers to the treatment of cancers with ionizing radiation. The term “chemotherapy” refers to the treatment of cancers using specific chemical agents. The term “targeted cancer therapy” refers to the treatment of cancers with agents (chemical compounds or macromolecules) that selectively interact with a chosen biomolecule.

In some embodiments, the additional therapy comprises administering a modulator of an immune checkpoint molecule.

As used herein, an “immune checkpoint” or “immune checkpoint molecule” is a molecule in the immune system that modulates a signal. An immune checkpoint molecule can be a co-stimulatory checkpoint molecule, i.e., turn up a signal, or an inhibitory checkpoint molecule, i.e., turn down a signal. A “co-stimulatory checkpoint molecule” as used herein is a molecule in the immune system that turns up a signal or is co-stimulatory. An “inhibitory checkpoint molecule”, as used herein is a molecule in the immune system that turns down a signal or is co-inhibitory.

As used herein, a “modulator of an immune checkpoint molecule” is an agent capable of altering the activity of an immune checkpoint in a subject. In certain embodiments, a modulator of an immune checkpoint molecule alters the function of one or more immune checkpoint molecules including PD-1, PD-L1, PD-L2, CTLA-4, TIM-3, LAG3, CD160, 2B4, TGF β, VISTA, BTLA, TIGIT, LAIR1, OX40, CD2, CD27, ICAM-1, NKG2C, SLAMF7, NKp80, CD160, B7-H3, LFA-1, 1COS, 4-1BB, GITR, CD30, CD40, BAFFR, HVEM, CD7, LIGHT, and CD83. The modulator of the immune checkpoint may be an activator (e.g., an agonist) or an inhibitor (e.g., an antagonist) of the immune checkpoint. In some embodiments, the modulator of the immune checkpoint molecule is an immune checkpoint binding protein (e.g., an antibody, antibody Fab fragment, divalent antibody, antibody drug conjugate, scFv, fusion protein, bivalent antibody, or tetravalent antibody). In some embodiments, the modulator of the immune checkpoint molecule is a monoclonal antibody or an antigen binding fragment thereof. In other embodiments, the modulator of the immune checkpoint molecule is a small molecule. In a particular embodiment, the modulator of the immune checkpoint molecule is an anti-PD1 antibody. In a particular embodiment, the modulator of the immune checkpoint molecule is an anti-PD-L1 antibody. In a particular embodiment, the modulator of the immune checkpoint molecule is an anti-CTLA-4 antibody.

In some embodiments, the modulator of the immune checkpoint molecule restores anti-tumor T-cell activity or blocks T-cell-inhibitory cell activity. In some embodiment, the modulator of the immune checkpoint molecule is an activator of the co-stimulatory checkpoint molecule, and the activator of co-stimulatory checkpoint molecule alters co-stimulatory signal required for full T-cell activation.

In some embodiments, the modulator of the immune checkpoint molecule is pembrolizumab, ipilimumab, nivolumab, atezolizumab, avelumab, durvalumab AGEN-1884, BMS-986016, CS-1002, LAG525, MBG453, MEDI-570, OREG-103/BY40, lirilumab, tremelimumab, pembrolizumab, nivolumab, AMP-224, AMP-514, BGB-A317, cemiplimab, JS001, PDR-001, CS-1001, PF-06801591, IBI-308, pidilizumab, SHR-1210, TSR-042, atezolizumab, avelumab, durvalumab, AMP-224, JS003, LY3300054, MDX-1105, SHR-1316, KN035, or CK-301.

In some embodiments, the modulator of the immune checkpoint molecule and the MDM2 inhibitor are administered concurrently. In some embodiments, the modulator of the immune checkpoint molecule and the MDM2 inhibitor are administered sequentially.

Diseases

In certain embodiments, the subject is with cancer. In certain embodiments, the subject is diagnosed with cancer or has a cancer condition. In certain embodiments, the subject is a cancer patient.

In certain embodiments, the cancer is solid tumor or hematologic malignancy. In various embodiments, the cancer is selected from the group consisting of leukemia, a lymphoma, a melanoma, a carcinoma, and a sarcoma. In certain embodiments, the cancer is selected from the group consisting of adrenal cortical cancer, advanced cancer, anal cancer, aplastic anemia, bile duct cancer, bladder cancer, bone cancer, bone metastasis, brain/CNS tumors in adults, brain/CNS tumors in children, breast cancer, breast cancer in men, cancer in children, cancer of unknown primary, Castleman disease, cervical cancer, colon/rectum cancer, endometrial cancer, esophagus cancer, Ewing family of tumors, eye cancer, gallbladder cancer, gastrointestinal carcinoid tumors, gastrointestinal stromal tumor (GIST), gestational trophoblastic disease, head and neck cancer, Hodgkin disease, Kaposi sarcoma, kidney cancer, laryngeal and hypopharyngeal cancer, leukemia-acute lymphocytic (ALL) in adults, leukemia-acute myeloid (AML), leukemia-chronic lymphocytic (CLL), leukemia-chronic myeloid (CML), leukemia-chronic myelomonocytic (CMML), leukemia in children, liver cancer, lung cancer-non-small cell, lung cancer-small cell, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, non-Hodgkin lymphoma in children, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma-adult soft tissue cancer, skin cancer-basal and squamous cell, skin cancer-melanoma, small intestine cancer, stomach cancer, testicular cancer, thymus cancer, thyroid cancer, uterine sarcoma, vaginal cancer, vulvar cancer, Waldenstrom macroglobulinemia, and Wilms Tumor. In one embodiment, the combination therapy is used to treat cancer selected from the group consisting of melanoma, Hodgkin lymphoma, lung cancer, kidney cancer, bladder cancer, head and neck cancer, Merkel cell carcinoma, urothelial carcinoma, solid tumors that are microsatellite instability-high or mismatch repair-deficient, sarcoma, colon cancer, prostate cancer, choriocarcinoma, breast cancer, retinoblastoma, stomach carcinoma, acute myeloid leukemia, lymphoma, multiple myeloma, or leukemia.

In certain embodiments, the cancer is selected from the group consisting of gastric cancer, gastrointerestinal stromal tumor, adenoid cystic carcinoma, cholangiocarcinoma, lung cancer, melanoma, breast cancer, colon cancer, ovian cancer, prostate cancer, liver cancer (e.g. hepatocellular carcinoma), bladder cancer, pancreatic cancer, renal cancer, esophageal cancer, head and neck cancer, thyroid cancer, cutaneous squamous cell carcinoma, glioblastoma. neuroblastoma, urinary bladder cancer, hysterocarcinoma, melanoma, osteosarcoma, lymphoma (e.g., mantel cell lymphoma, diffuse large B cell lymphoma), leukemia (e.g., T-cell prolymphocytic leukemia, chronic lymphocytic leukemia, or acute myeloid leukemia), multiple myeloma, ulterine cancel, colorectal cancer, lung adenocarcinoma, uterine carcinosarcoma CS, lung squamous cell carcinoma, cervical cancer, esophagus cancer, sarcoma, chromophobe, renal cell carcinoma (RCC), clear cell RCC, papillary RCC, uveal melanoma, testicular germ cell, low grade glioma (LGG), mesothelioma, pheochromocytoma and paraganglioma (PCPG), or thymoma.

In certain embodiments, the cancer is locally advanced or metastatic solid tumor or lymphoma. In certain embodiments, the subject is treatment-experienced and shows disease progression. “Treatment-experienced” as used herein means that the subject has been treated with an anti-cancer therapy. Disease progression can be characterized by a sign of reduced responsiveness to the previous treatment, for example, increase in tumor size, increase in tumor cell number, or tumor growth.

In one embodiment, administration of an MDM2 inhibitor (e.g., Compound C) as described herein results in one or more of, reducing tumor size, weight or volume, increasing time to progression, inhibiting tumor growth and/or prolonging the survival time of a subject having cancer.

### V. Kits

In another aspect, the present disclosure further provides a kit for use in the methods described herein. In certain embodiments, the kit comprises one or more of reagents, such as the primers, the probes, and/or the antibodies, or microarray provided herein. The primers, the probes, and/or the antibodies may or may not be detectably labeled. In certain embodiments, the kits may further comprise other reagents to perform the methods described herein. In such applications the kits may include any or all of the following: suitable buffers, reagents for isolating nucleic acid, reagents for amplifying the nucleic acid (e.g. polymerase, dNTP mix), reagents for hybridizing the nucleic acid, reagents for sequencing the nucleic acid, reagents for quantifying the nucleic acid (e.g. intercalating agents, detection probes), reagents for isolating the protein, and reagents for detecting the protein (e.g. secondary antibody). Typically, the reagents useful in any of the methods provided herein are contained in a carrier or compartmentalized container. The carrier can be a container or support, in the form of, e.g., bag, box, tube, rack, and is optionally compartmentalized.

In one embodiment, the kit comprises: one or more reagents for detecting presence of one or more inactivating mutation in ATM and/or ATR; or one or more reagents for measuring expression level of ATM and/or ATR. In one embodiment, the kit comprises: one or more reagents for measuring copy number variation of MDM2, or one or more reagents for measuring expression level of MDM2. In one embodiment, the kit further comprises one or more reagents for detecting presence or absence of a functional p53 (e.g. wild-type p53).

In one aspect, the present disclosure provides reagents for detecting presence of one or more inactivating mutation in ATM and/or ATR, measuring expression level of ATM and/or ATR, measuring copy number variation of MDM2, measuring expression level of MDM2, and/or detecting presence or absence of a functional p53 (e.g. wild-type p53). The measurement or detection can be at RNA level, DNA level and/or protein level. Suitable reagents for detecting target RNA, target DNA or target proteins can be used. In certain embodiments, the detection reagents comprise primers or probes that can hybridize to the polynucleotide of ATM, ATR, MDM2 or p53. In certain embodiments, the detection reagents comprise antibodies that can specifically bind to the protein of ATM, ATR, MDM2 or p53.

The term “primer” as used herein refers to oligonucleotides that can specifically hybridize to a target polynucleotide sequence, due to the sequence complementarity of at least part of the primer within a sequence of the target polynucleotide sequence. A primer can have a length of at least 8 nucleotides, typically 8 to 70 nucleotides, usually of 18 to 26 nucleotides. For proper hybridization to the target sequence, a primer can have at least 75%, at least 80%, at least 85%, at least 90%, or at least 95% sequence complementarity to the hybridized portion of the target polynucleotide sequence. Oligonucleotides useful as primers may be chemically synthesized according to the solid phase phosphoramidite triester method first described by Beaucage and Caruthers, Tetrahedron Letts. (1981) 22: 1859-1862, using an automated synthesizer, as described in Needham-Van Devanter et al, Nucleic Acids Res. (1984) 12:6159-6168.

Primers are useful in nucleic acid amplification reactions in which the primer is extended to produce a new strand of the polynucleotide. Primers can be readily designed by a skilled artisan using common knowledge known in the art, such that they can specifically anneal to the nucleotide sequence of the target nucleotide sequence of the at least one biomarker provided herein. Usually, the 3′ nucleotide of the primer is designed to be complementary to the target sequence at the corresponding nucleotide position, to provide optimal primer extension by a polymerase.

The term “probe” as used herein refers to oligonucleotides or analogs thereof that can specifically hybridize to a target polynucleotide sequence, due to the sequence complementarity of at least part of the probe within a sequence of the target polynucleotide sequence. Exemplary probes can be, for example DNA probes, RNA probes, or protein nucleic acid (PNA) probes. A probe can have a length of at least 8 nucleotides, typically 8 to 70 nucleotides, usually of 18 to 26 nucleotides. For proper hybridization to the target sequence, a probe can have at least 75%, at least 80%, at least 85%, at least 90%, or at least 95% sequence complementarity to hybridized portion of the target polynucleotide sequence. Probes and also be chemically synthesized according to the solid phase phosphoramidite triester method as described above. Methods for preparation of DNA and RNA probes, and the conditions for hybridization thereof to target nucleotide sequences, are described in Molecular Cloning: A Laboratory Manual, J. Sambrook et al., eds., 2nd edition. Cold Spring Harbor Laboratory Press, 1989, Chapters 10 and 11.

The term “antibody” as used herein refers to an immunoglobulin or an antigen-binding fragment thereof, which can specifically bind to a target protein antigen. Antibodies can be identified and prepared by selection of antibodies from libraries of recombinant antibodies in phage or similar vectors, as well as preparation of polyclonal and monoclonal antibodies by immunizing animals such as rabbits or mice (see, e.g., Huse et al., Science (1989) 246:1275-1281; Ward et al, Nature (1989) 341:544-546).

In certain embodiments, the primes or probes provided herein comprise a polynucleotide sequence hybridizable to a portion within the sequence of SEQ ID NO: 1, 3, 5 or 7. In certain embodiments, the primes or probes provided herein comprise a polynucleotide sequence having at least 60%, 65%, 70%, 75%, 80%, 85%, 90%, 95%, 97%, 98%, 99% or 100% complementarity to a portion within the sequence of SEQ ID NO: 1, 3, 5 or 7. In certain embodiments, the antibodies provided herein comprise an antigen-binding region capable of specifically binding to an epitope within the protein or polypeptide having the sequence of SEQ ID NO: 2, 4, 6 or 8.

In certain embodiments, the primers, the probes and the antibodies provided herein are detectably labeled. Examples of the detectable label suitable for labeling primers, probes and antibodies include, for example, chromophores, radioisotopes, fluorophores, chemiluminescent moieties, particles (visible or fluorescent), nucleic acids, ligand, or catalysts such as enzymes.

Examples of radioisotopes include, without limitation, ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P.

Examples of fluorophores include, without limitation, Acridine, 7-amino-4-methylcoumarin-3-acetic acid (AMCA), BODIPY, Cascade Blue, Cy2, Cy3, Cy5, Cy7, Edans, Eosin, Erythrosin, Fluorescein, 6-FAM, TET, JOC, HEX, Oregon Green, Rhodamine, Rhodol Green, Tamra. Rox, and Texas Red™ (Molecular Probes, Inc., Eugene, Oreg.).

Examples of enzymes include, without limitation, alkaline phosphatase, acid phosphatase, horseradish peroxidase, beta-galactosidase, and ribonuclease.

Examples of ligands include, without limitation, biotin, avidin, an antibody or an antigen.

It should be understood that it is not necessary for a detectable label to produce a detectable signal, for example, in some embodiments, it may can react with a detectable partner or react with one or more additional compounds to generate a detectable signal. For example, the detectable label can be a ligand capable of functioning as a specific binding pair member for a labeled ligand (e.g. a secondary labeled antibody). For another example, enzymes are useful detectable labels due to their catalytic activity to catalyze chromo-, fluoro-, or lumo-genic substrate which results in generation of a detectable signal.

In certain embodiments, the detectably labeled primers, probes or antibodies as provided herein can further comprise a quencher substance. A quencher substance refers to a substance which, when present in sufficiently close proximity to a fluorescent substance, can quench the fluorescence emitted by the fluorescent substance as a result of, for example, fluorescence resonance energy transfer (FRET).

Examples of a quencher substance include, without limitation, Tamra, Dabcyl, or Black Hole Quencher (BHQ, Biosearch Technologies), DDQ (Eurogentec), Iowa Black FQ (Integrated DNA Technologies), QSY-7 (Molecular Probes), and Eclipse quenchers (Epoch Biosciences).

Primer and probes can be labeled to high specific activity by either the nick translation method or by the random priming method. Useful probe labeling techniques are described in the literature (Fan, Y-S, Molecular cytogenetics: protocols and applications, Humana Press, Totowa, N.J. xiv, 411 (2002)).

In addition, the kits may include instructional materials containing directions (i.e., protocols) for the practice of the methods provided herein. While the instructional materials typically comprise written or printed materials they are not limited to such.

In certain embodiments, the kits can further comprise a computer program product stored on a computer readable medium. When computer program product is executed by a computer, it performs the step of identifying the subject as likely to respond to the treatment with an MDM2 inhibitor based on: i) the deficiency of ATM and/or ATR, or ii) the gain in MDM2, or both i) and ii), found in the biological sample. Any medium capable of storing such computer executable instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The computer programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network.

In some embodiments, the present disclosure provides oligonucleotide probes attached to a solid support, such as an array slide or chip, e.g., as described in Eds., Bowtell and Sambrook DNA Microarrays: A Molecular Cloning Manual (2003) Cold Spring Harbor Laboratory Press. Construction of such devices are well known in the art, for example as described in US Patents and Patent Publications U.S. Pat. No. 5,837,832; PCT application WO95/11995; U.S. Pat. Nos. 5,807,522; 7,157,229, 7,083,975, 6,444,175, 6,375,903, 6,315,958, 6,295,153, and 5,143,854, 2007/0037274, 2007/0140906, 2004/0126757, 2004/0110212, 2004/0110211, 2003/0143550, 2003/0003032, and 2002/0041420. Nucleic acid arrays are also reviewed in the following references: Biotechnol Annu Rev (2002) 8:85-101; Sosnowski et al. Psychiatr Genet (2002)12(4): 181-92; Heller, Annu Rev Biomed Eng (2002) 4: 129-53; Kolchinsky et al., Hum. Mutat (2002) 19(4):343-60; and McGail et al., Adv Biochem Eng Biotechnol (2002) 77:21-42.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXEMPLIFICATION

While we have described a number of embodiments of this invention, it is apparent that our basic examples may be altered to provide other embodiments that utilize the methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

### Example 1. Development of Predictive Biomarker for Compound C

Objective of the Study

The objective of this study was to evaluate the in vivo anti-tumor efficacy of Compound C single agent in the MDM2 amplification (amp) subcutaneous PDX model in BALB/c nude mice.

1. Generation of the Patient Derived Xenograft (PDX) Model

The PDX models were originally established from a surgically resected clinical sample, and implanted in nude mice defined as passage 0 (P0). The next passage implanted from P0 tumor was defined as passage1 (P1), and so on during continual implantation in mice. The P3-P7 tumor tissue will be used for the study. A list of 10 PDX models and their corresponding test article is shown in the experimental design table (Table 1 and Table 2).

TABLE 1
Description of 10 PDX models

| Model Name | Cancer type |
|---|---|
| ST-02-0075 | Gastric cancer |
| ST-02-0164 | Gastric cancer |
| ST-02-0203 | Gastric cancer |
| CH-17-0044 | Cholangiocarcinoma |
| LU-01-0566 | lung cancer |
| LU-01-0448 | lung cancer |
| LU-01-0582 | lung cancer |
| CC6658 | Cholangiocarcinoma |
| LU0861 | Lung Cancer |
| ME2194 | Melanoma |

TABLE 2
Description of experimental design

| | | | Dose | Dose Volume | Dose | |
|---|---|---|---|---|---|---|
| Group | n | Treatment | (mg/kg) | (mL/kg) | Route | Schedule |
| 1 | 2 | Vehicle | — | 10 | p.o | Q2D × |
| | | Control | | | | 3W |
| 2 | 2 | Compound C | 200 | 10 | p.o | Q2D × |
| | | | | | | 3W |

Note:
p.o., orally.
Q2D, every two days;
W, week

2. Tumor Inoculation and Animal Grouping

Each mouse will be implanted subcutaneously at the right flank with the tumor slices (˜30 mm³) for tumor development. For gastric cancer, tumor slices will be implanted directly into Balb/c nude mice. The animals will be randomized and treatment will be started when the average tumor size reaches approximately 150-200 mm³ for the efficacy study. For cholangiocarcinoma and lung cancer models, tumor slices will be first implanted into NOD SCID mice, when tumors grow to proper size, they will be passaged to Balb/c nude mice. The animals will be randomized and treatment will be started when the average tumor size reaches approximately 150-200 mm³ for the efficacy study. The test article administration and the animal numbers in each group are shown in the following experimental design Table 3

3. Testing Article Preparation

TABLE 2
Description of test article preparation

| | | | Conc. | |
|---|---|---|---|---|
| Compounds | Package | Preparation | mg/mL | Storage |
| Vehicle | — | 0.2% HPMC | — | RT |
| Compound | 2.011g/ | Grind 160 mg Compound C | 20 | 4° C. |
| C | package | in 8 mL 0.2% HPMC to | | |
| | | make homogenous suspension | | |

Note:
Ensure that formulation is homogenous immediately before use by gently turning the tube up and down.

4. Gene Mutation Detection

TP53, MDM2, and ATM gene status were analyzed by whole exon sequencing (WES), RNA sequencing (RNA-seq) or microarrays. For the copy number variation (CNV) of MDM2, a CNV>3 was defined as amplification.

5. Tumor Measurements and Endpoints

The major endpoint was to see if the tumor growth could be delayed or mice could be cured. Tumor size was measured twice weekly in two dimensions using a caliper, and the volume was expressed in mm³ using the formula: V=0.5a×b² where a and b are the long and short diameters of the tumor, respectively. The tumor size was then used for calculations of both TGI and T/C values. TGI was calculated for each group using the formula: TGI (%)=[1−(Ti−T0)/(Vi−V0)]×100; Ti is the average tumor volume of a treatment group on a given day, T0 is the average tumor volume of the treatment group on the day of treatment start, Vi is the average tumor volume of the vehicle control group on the same day with Ti, and V0 is the average tumor volume of the vehicle group on the day of treatment start. The T/C value (in percent) is an indication of antitumor effectiveness; T and C are the mean volumes of the treated and control groups, respectively, on a given day.

Result

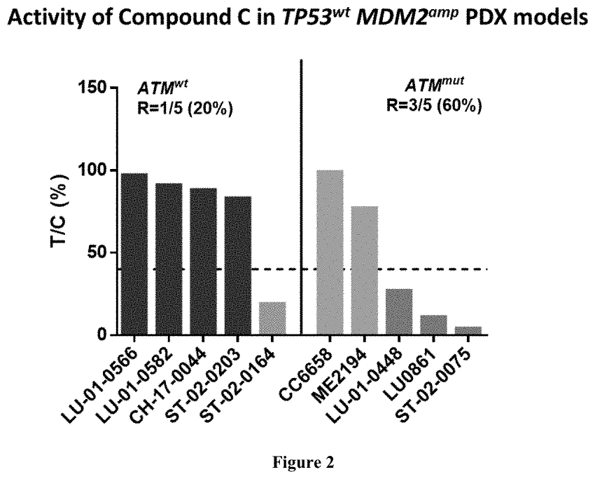

The overall response rate (ORR) in all 10 PDX model was 4/10(40%). In TP53^{wt}/MDM2^{map}/ATM^{mut} PDX models, the response rate to Compound C was 60%, while in TP53^{wt}/MDM2^{amp}/ATM^{wt}PDX models, the response rate was 20% (Table 4). Thus, ATM mutations may further differentiate the responders to Compound C in MDM2^{amp}/p53^{wt} PDX models (FIG. 2).

TABLE 4
Tumor growth inhibition of Compound C on each PDX model

| | | Cancer | Gene | ATM | Ave | Responder |
|---|---|---|---|---|---|---|
| Model | | type | background | mutation | TGI % | (TGI % >60%) |
| 1 | ST-02-0075 | Gastric | MDM2^{amp}/ | p.H1380Y | 96 | Yes |
| | | | p53^{wt} | | | |
| 2 | LU0861 | Lung | MDM2^{amp}/ | p.N1983S | 89 | Yes |
| | | | p53^{wt} | | | |
| 3 | LU-01-0448 | Lung | MDM2^{amp}/ | p.H1380Y | 73 | Yes |
| | | | p53^{wt} | | | |
| 4 | ME2194 | Melanoma | MDM2^{amp}/ | p.N1983S | 23 | No |
| | | | p53^{wt} | | | |
| 5 | CC6658 | Cholangio- | MDM2^{amp}/ | p.N1983S | 1 | No |
| | | carcinoma | p53^{wt} | | | |
| 6 | ST-02-0164 | Gastric | MDM2^{amp}/ | Wild-type | 81 | Yes |
| | | | p53^{wt} | | | |
| 7 | ST-02-0203 | Gastric | MDM2^{amp}/ | Wild-type | 17 | No |
| | | | p53^{wt} | | | |
| 8 | CH-17-0044 | Cholangio- | MDM2^{amp}/ | Wild-type | 12 | No |
| | | carcinoma | p53^{wt} | | | |
| 9 | LU-01-0582 | Lung | MDM2^{amp}/ | Wild-type | 9 | No |
| | | | p53^{wt} | | | |
| 10 | LU-01-0566 | Lung | MDM2^{amp}/ | Wild-type | 3 | No |
| | | | p53^{wt} | | | |

Note:
wt indicates wild-type; amp indicates amplification.

### Example 2. Development of Predictive Biomarker for Compound C

The objective of this study was to evaluate the in vivo anti-tumor efficacy of Compound C single agent in the 10 TP53 wild-type (wt) and MDM2 normal subcutaneous PDX model in BALB/c nude mice.

The experimental methods and procedures are similar to those described in Example 1. Experimental design is shown in Table 5.

TABLE 5
Description of experimental design

| | | | Dose | Dose Volume | Dose | |
|---|---|---|---|---|---|---|
| Group | n | Treatment | (mg/kg) | (mL/kg) | Route | Schedule |
| 1 | 2 | Vehicle | — | 10 | p.o | QD × |
| | | Control | | | | 2W |
| 2 | 2 | Compound C | 100 | 10 | p.o | QD × |
| | | | | | | 2W |

Note:
p.o., orally.
QD, every day;
W, week

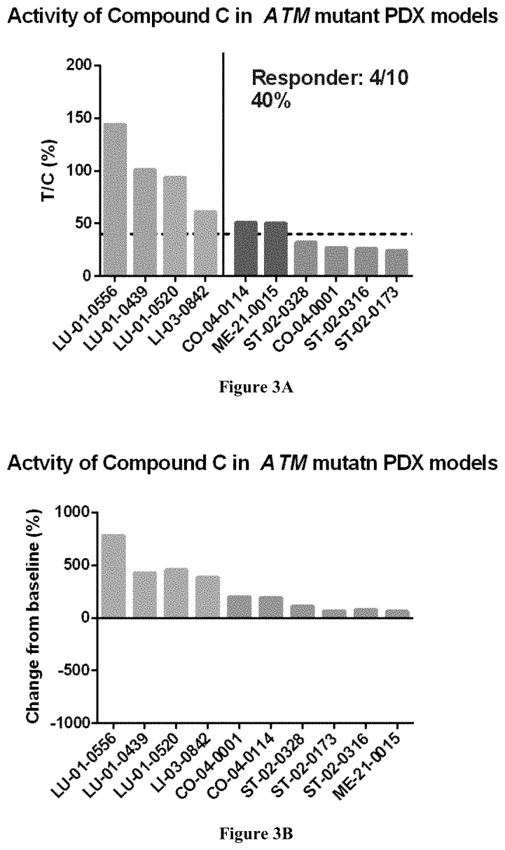

The interim results (Table 6 and FIGS. 3A, and 3B) showed that the overall response rate (ORR) in all 10 PDX model was 4/10 (400%).

TABLE 6
Tumor growth inhibition of Compound C on each PDX model

| | | ATM | ATR | | |
|---|---|---|---|---|---|
| Model | Cancer type | mutation | mutation | TGI % | T/C % |
| ST-02- | Gastric cancer | p.N2875S | wt | 76 | 24 |
| 0173 | | | | | |
| ST-02- | Gastric cancer | p.H1380Y | wt | 74 | 26 |
| 0316 | | | | | |
| CO-04- | Colon cancer | p.R2598Q | p.K243T | 73 | 27 |
| 0001 | | | | | |
| ST-02- | Gastric cancer | p.1599_16 | wt | 68 | 32 |
| 0328 | | 00del | | | |
| ME-21- | Melanoma | p.H1380Y | wt | 50 | 50 |
| 0015 | | | | | |
| CO-04- | Colon cancer | p.H1380Y | wt | 49 | 51 |
| 0114 | | | | | |
| LI-03-0842 | Liver cancer | p.I2865V | wt | 39 | 61 |
| LU-01-052 | Lung cancer | p.Q476R | wt | 6 | 94 |
| LU-01- | Lung cancer | p.N1650S | wt | −1 | 101 |
| 0439 | | | | | |
| LU-01- | Lung cancer | p.R2741T | wt | −44 | 144 |
| 0556 | | | | | |

Note:
wt indicates wild-type.

### Example 3. In Vitro Cellular Activity of Compound C in Cancer Cell Lines

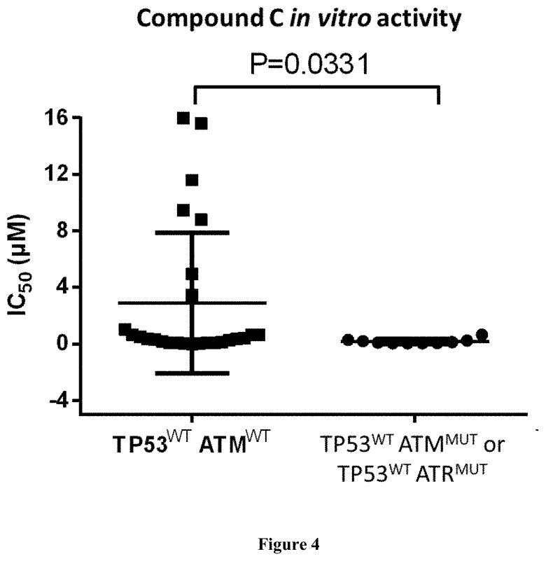

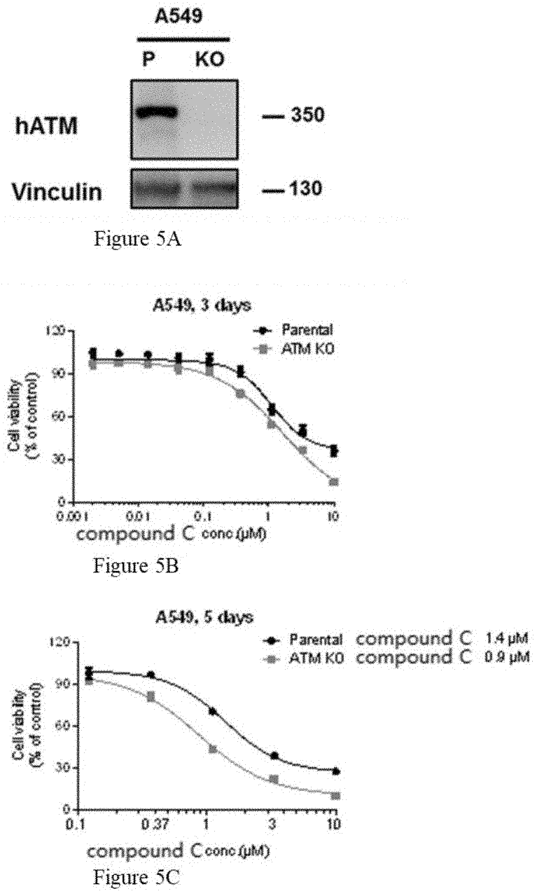

Activity of Compound C is tested in various cancer cell lines with indicated TP53, ATM and ATR genetic status. The activity (IC50 in uM) of Compound C in TP53^{WT} ATM^{WT} cell lines versus TP53^{WT}ATR^{MUT} or TP53^{WT}ATM^{MUT} cell lines was plotted (Figure. 4, MUT indicated mutant; WT indicated Wild-type). FIG. 4 shows that TP53^{WT} ATR^{MUT} or TP53^{WT}ATM^{MUT} cell lines have higher sensitivity to Compound C than TP53^{WT}ATM^{WT} cell lines. The copy number of MDM2 and the expression level of MDM2 mRNA in the exemplary cancer cell lines having an IC50 of Compound C less than 0.3 uM are further determined and results are shown in Table 7.

TABLE 7

| Cancer | Cell | IC50 (uM) | | | P53 | | | MDM2 | |
|---|---|---|---|---|---|---|---|---|---|
| type | line | Test#1 | Test#2 | Avg | Type | ATM | ATR | CNV > 3 | RNAseq |
| Neuro- | IMR-32 | 0.085 | 0.102 | 0.0935 | WT | p.V2716A, | WT | no | 4.852414 |
| blastoma | | | | | | p.K1903fs | | | |
| | SK-N-SH | 0.067 | 0.06 | 0.0635 | WT | WT | WT | no | 4.576566 |
| Breast cancer | ZR-75-1 | 0.0109 | 0.0354 | 0.02315 | WT | WT | WT | no | 5.755088 |
| | ZR-75-30 | 0.109 | 0.301 | 0.205 | WT | WT | p.Q1926H | no | 3.676502 |
| Gastric cancer | AGS | 0.01 | 0.02 | 0.015 | WT | WT | WT | no | 5.352527 |
| | MKN-45 | 0.097 | 0.057 | 0.077 | WT | WT | WT | no | 6.110541 |
| Kidney cancer | ACHN | 0.239 | 0.34 | 0.2895 | WT | p.K1903fs | WT | no | 5.270044 |
| Hepatocellular | HepG2 | 0.254 | 0.231 | 0.2425 | WT | p.V2906I | WT | no | 4.385168 |
| carcinoma | | | | | | | | | |
| Colon cancer | HCT116 | 0.055 | 0.057 | 0.056 | WT | p.A1127V | WT | no | 4.907641 |
| | LS 174T# | 0.0835 | 0.0769 | 0.0802 | WT | WT | WT | no | NA |
| | RKO | 0.13 | 0.11 | 0.12 | WT | WT | WT | no | 5.430499 |
| | LoVo | 0.069 | 0.026 | 0.0475 | WT | WT | WT | no | 5.095891 |
| Prostate cancer | LNCap | 0.044 | 0.039 | 0.0415 | WT | p.Q912* | p.S1493S; | no | 4.141814 |
| | | | | | | | p.K1379N | | |
| Urinary | HT-1197 | 0.213 | 0.156 | 0.1845 | WT | WT | WT | no | 6.378388 |
| bladder cancer | | | | | | | | | |
| Melanoma | A375 | 0.401 | 0.124 | 0.2625 | WT | WT | WT | no | 4.375418 |
| Osteosarcoma | SJSA-1 | 0.058 | 0.041 | 0.0495 | WT | WT | p.L1483F | yes | 8.050669 |
| Leukemia | MOLT-4 | 0.064 | 0.053 | 0.0585 | WT | p.S2165F, | WT | no | NA |
| | | | | | | p.G2919G | | | |
| | MV-4-11 | 0.108 | NA | 0.108 | WT | WT | WT | no | 2.608113 |
| Lymphoma | BC-3 | 0.181 | 0.0858 | 0.1334 | WT | p.H1083Y | WT | no | 2.610339 |

Note:
WT indicates wild-type; NA indicates not available.

### Example 4. A Phase Ib/II Study of Compound C in Combination with Pembrolizumab in Patients with Unresectable or Metastatic Melanomas or Advanced Solid Tumors

The Ib/II study consists of two parts, a dose escalation study and a Simon two-stage phase II study. In the dose escalation study, Compound C is combined with pembrolizumab of for the treatment of patients with metastatic solid tumor who failed their previous standard treatments. Four dose levels of Compound C are tested: 50, 100, 150, and 200 mg. Compound C is administrated orally every other day (QOD) for consecutive 2 weeks of a 21-day-cycle. Pembrolizumab is administrated at 200 mg IV on day1 of a 21-day-cycle. The primary objectives of the dose escalation study is to determine safety, tolerability, and determination of MTD and RP2D.

Results

A total of 14 patients in dose escalation study had been treated in 4 cohorts of Compound C (50 mg, 100 mg, 150 mg, 200 mg) in combination with pembrolizumab. One patient with advanced ovarian cancer (ATM germline mutation) in 100 mg cohort has received “confirmed complete regression (CR)” (still ongoing); another patient with advanced NSCLC in 100 mg cohort has received “confirmed partial regression (PR)” (still ongoing) after failed prior 6 lines therapies including 3 months' nivolumab treatment; 5 patients got “SD” after two cycle treatments, 2 of them has received “confirmed stable disease (SD)” (still ongoing). PK analysis indicated an approximately dose proportional increase in exposure across dose levels from 50 to 100 mg. Preliminary PD results showed that serum MIC-1 levels were elevated after Compound C treatment, suggesting a potential p53 activation in patients.

Conclusion

Compound C has shown promising anti-tumor effects in several tumor types when in combination with pembrolizumab.

### Example 5. Genetic Knock-Out of ATM Gene in A549 Cells Increases Cellular Sensitivity to Treatment of Compound C In Vitro

Methods

1. CTG Assay

The anti-proliferative effects of Compound C in parental and ATM knock-out A549 cell lines were quantitatively determined by measuring ATP via the Cell Titer-Glo® luminescence cell viability assay kit. Cells were seeded in 96-well plates and treated with different concentrations of the test agents as indicated. Compound C was tested by using up to 8 series of concentrations. Triplicated wells were employed at each concentration.

Briefly, the procedures were as followed. First, cells were grown to the logarithmic phase and collected by centrifugation. Cells were re-suspended, counted and diluted to the desired concentration. Cells were mixed well, and 90 μL of the cell suspension (8×10³ cells) was added to each well in a 96-well plate, and incubated at a 37° C. incubator with 5% CO₂ overnight. Three or more blank control wells containing only medium (100 μL/well) without cells were placed in the same plate to obtain background luminescence signals.

Compound C was serially diluted with a 1:3 ratio to obtain 5-8 series concentration, and 10 μL/well of diluted Compound C solution was added into a 96-well plate. The plates were incubated at 37° C. in a 5% CO₂ incubator for 3 days or 5 days. Cell growth was observed daily under an inverted microscope.

At the end of the treatment, the 96-well plates were removed from the incubator and equilibrated to room temperature and then 30 μL of Cell Titer-Glo® Reagent (protected from light) was added to each well. The 96-well plates were placed on an orbital shaker and cells were mixed well with the reagents for 2 minutes to make cells lysis. The 96-well plates were kept at room temperature for another 10 minutes to stabilize the luminescent signal. Luminescence signals were then detected using a Biotek synergy H1 microplate reader. Using the average fluorescence signal values of the three replicate wells, the percentage of cell viability was calculated by the following formula:

Percentage of cell viability (%)=(test cell fluorescence signal value−negative control cell fluorescence signal value)/(control cell fluorescence signal value−negative control cell fluorescence signal value)×100%.

IC₅₀ was calculated using a non-linear regression data analysis method of Graphpad Prism 6.0 software (Golden software, Golden, Colo., USA) and cell survival curves were plotted.

2. Flow Cytometry Analysis of Apoptosis

Apoptosis was detected using an Annexin V-PI (propidium iodide) staining kit. Briefly, cells were harvested 72 hours after the treatment and washed with PBS. Cells were then stained with Annexin-V and PI, analyzed by an Attune N×T flow cytometer following manufacturer's instruction. Apoptosis data were obtained by analyzing 20,000 cells from each experimental condition.

Results

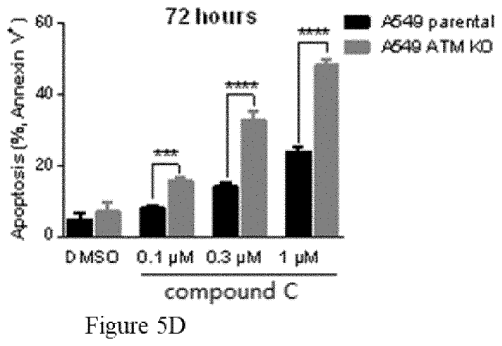

To examine the effects of ATM on activity of Compound C, ATM gene in A549 cells was knocked out by CRISPR/Cas9 technique following methods adapted from Ran A F et al., Cell, (2013)154:1380-1389 and Cho S W et al., Nature Biotechnology, (2013)31:230-232. After verification of successful knock-out of ATM gene (FIG. 5A, p indicating parental cells with ATM gene, KO indicating cells with ATM gene knocked out), the effect of ATM loss on cellular activity of Compound C was examined. As shown in FIG. 5B-C, A549 ATM KO cells are more sensitive to Compound C treatment in vitro (Compound C showing IC50 of 1.4 μM in parental cells vs. IC50 of 0.9 μM in ATM knock-out cells after 5 days treatment). In addition, Compound C induces more cell apoptosis in A549 ATM KO cells than in parental cells (FIG. 5D).

### Example 6. A549 ATM KO Cells have Higher ROS Levels than Parental Cells, and COMPOUND C Treatment Results in More ROS Induction in ATM KO Cells

Methods: Flow Cytometry Analysis of ROS Production

ROS production was detected using a reactive oxygen species (ROS) assay kit (Beyotime, Cat. #S0033), which uses the fluorescent probe DCFH-DA to detect ROS. Cells were collected 48 hours after treatment and loaded with fluorescent probes according to the kit instructions. Fluorescence intensity was measured using an Attune N×T flow cytometer.

Results

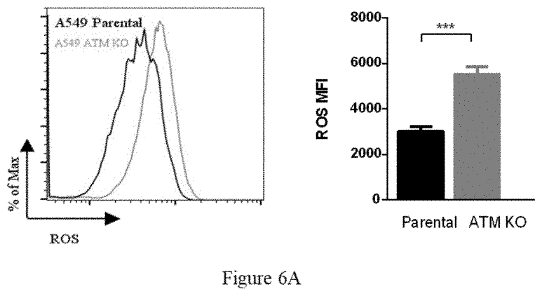

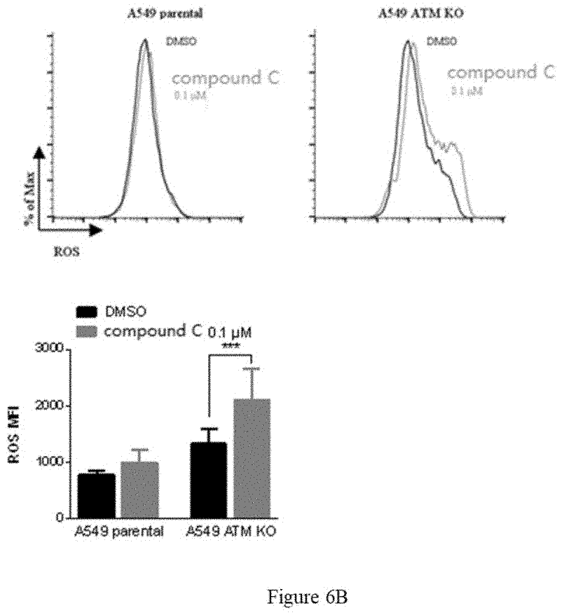

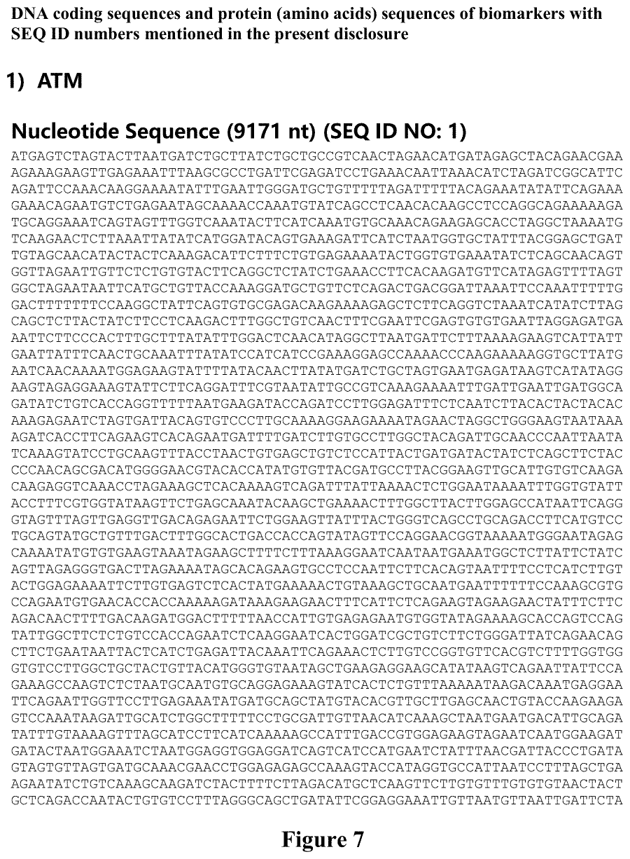

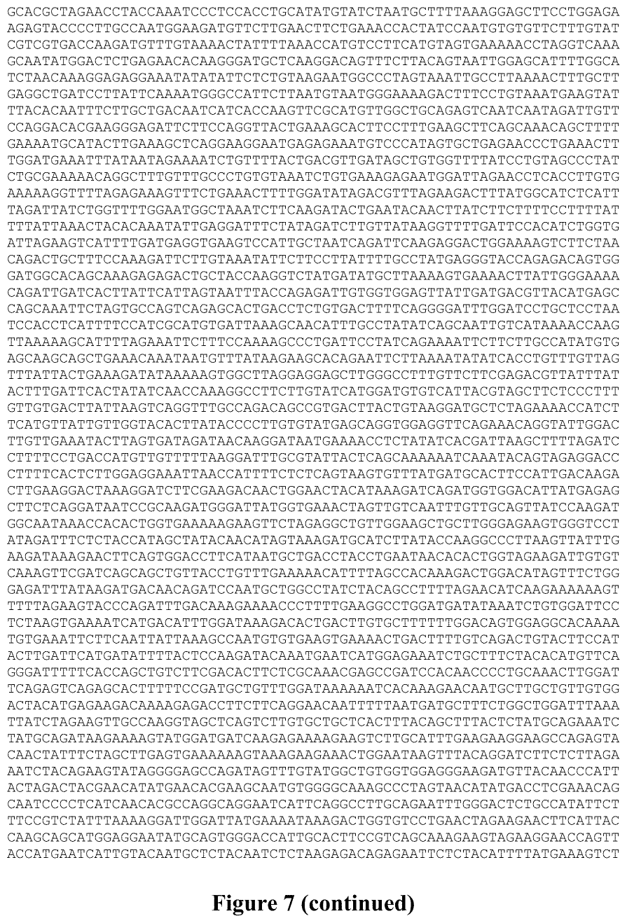

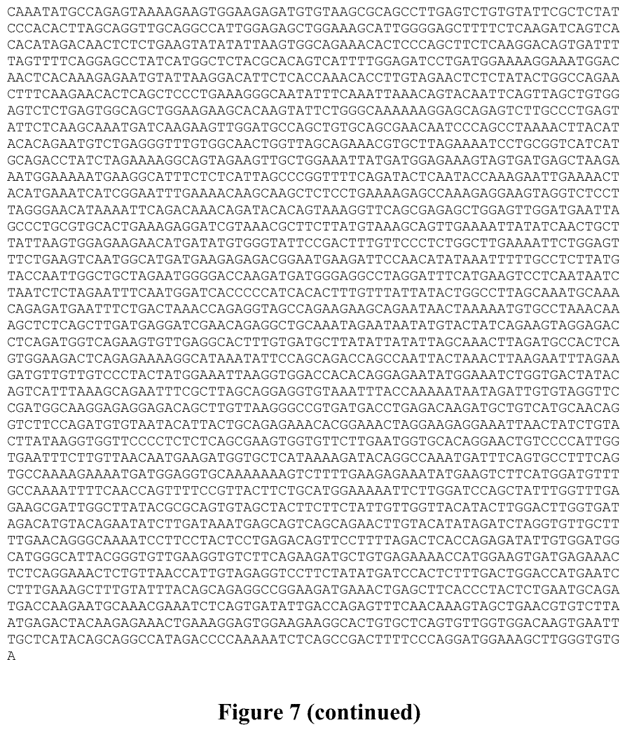

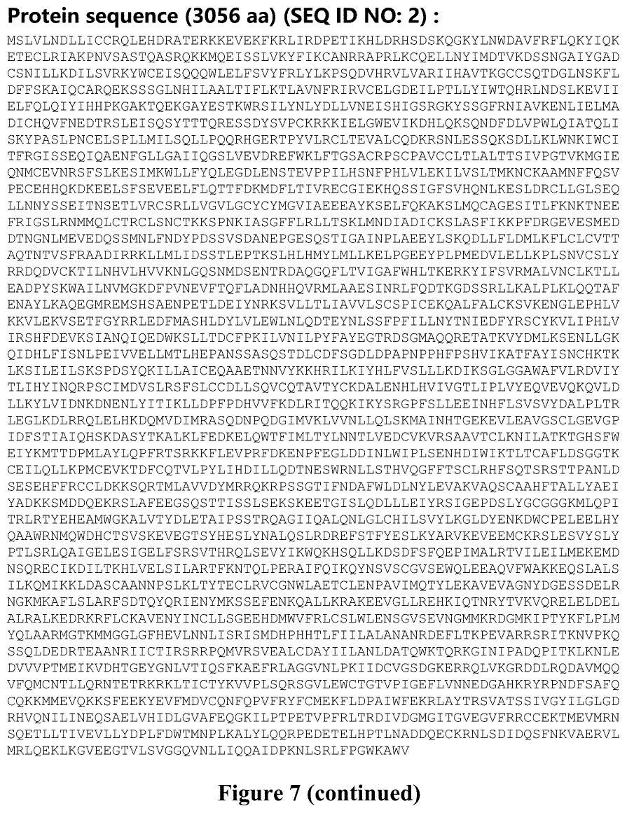

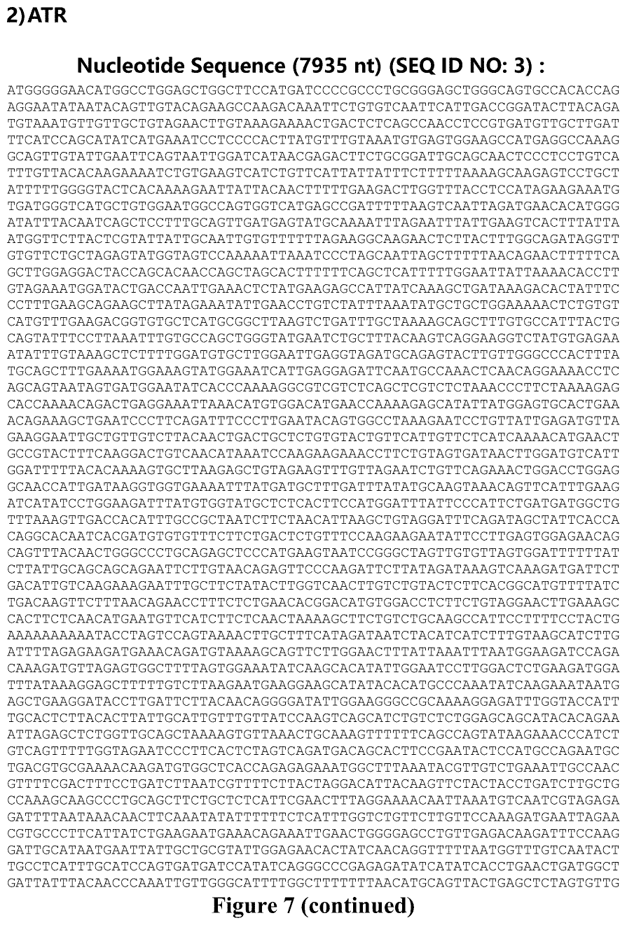

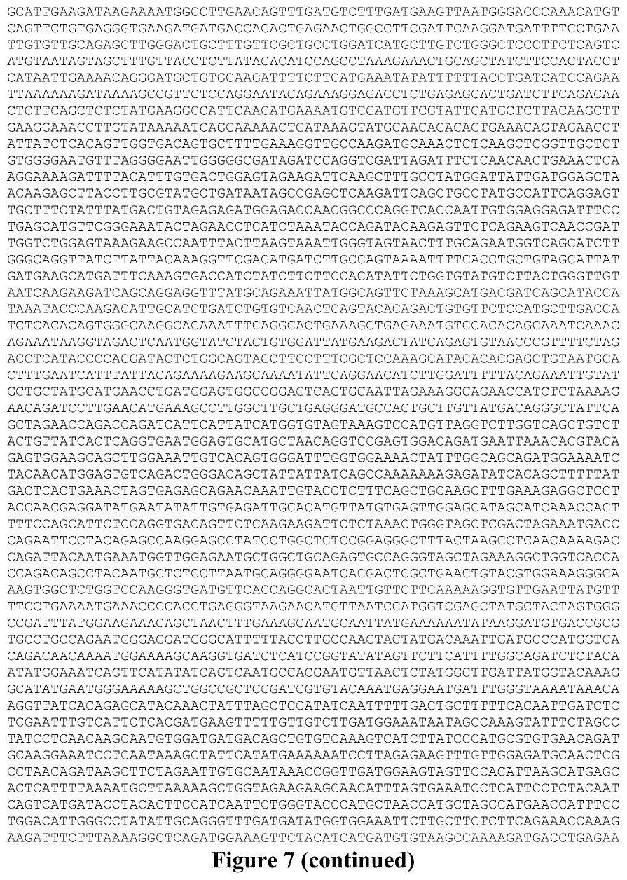

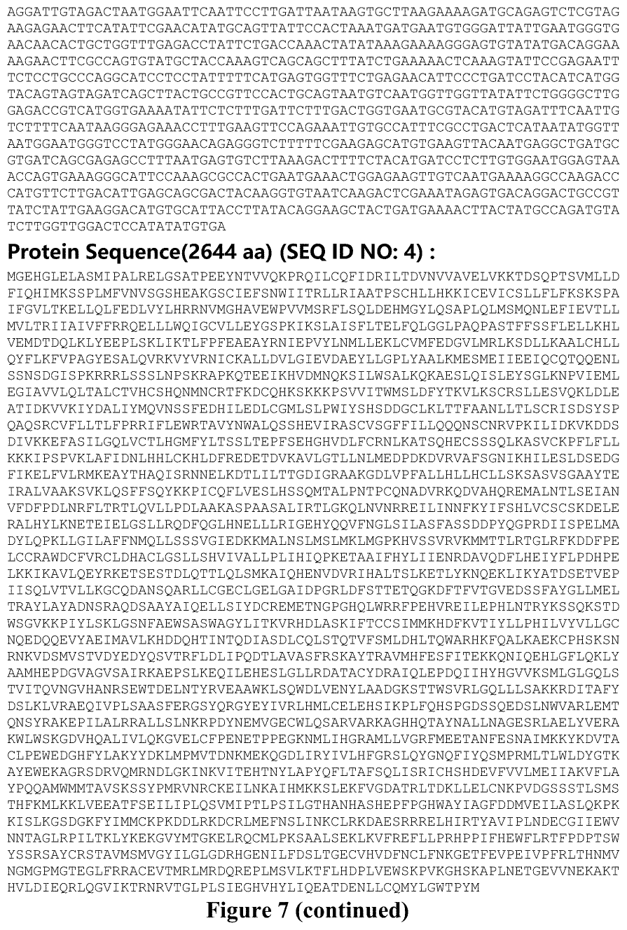

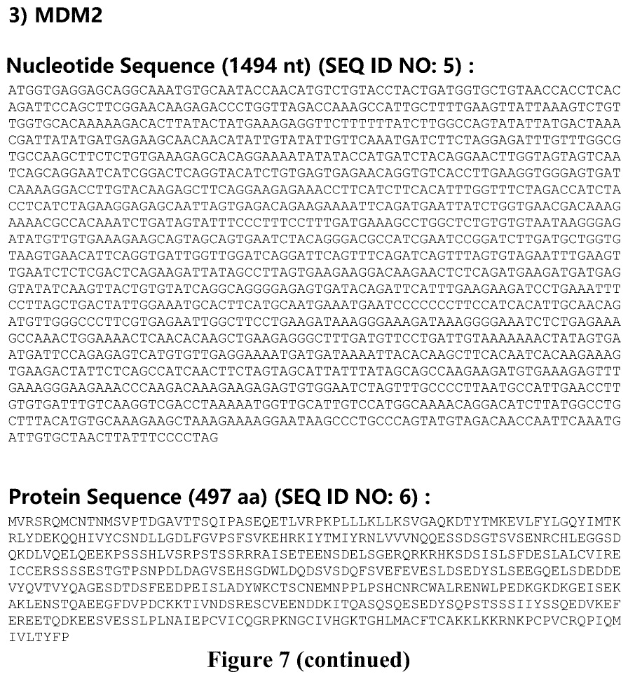

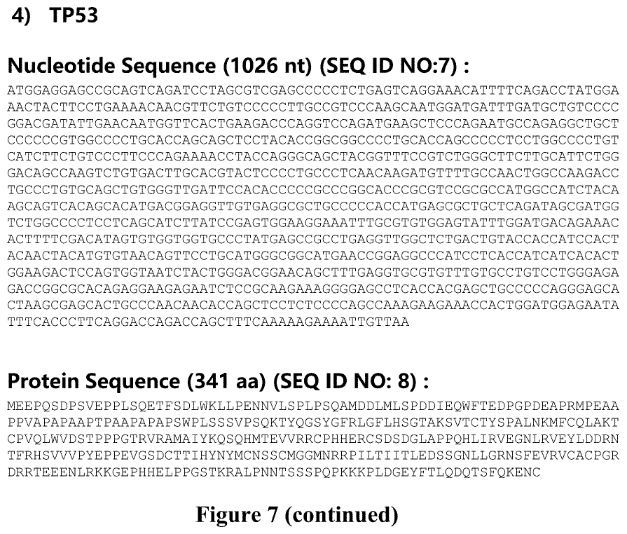

A549 ATM knock-out cells have higher baseline ROS levels than parental cells (FIG. 6A). Compound C treatment results in more ROS induction in ATM KO cells, compared to parental cells (FIG. 6B). ROS is a known apoptosis inducer. These data indicate that ATM knock-out resulted in increased ROS level, and thus lowered the apoptosis threshold for Compound C.

What is claimed is:

1. A method of identifying a subject with cancer as likely to respond to treatment with an MDM2 (Murine Double Minute 2) inhibitor, the method comprising:
a) providing a biological sample from the subject;
b) determining in the biological sample:
i. if there is deficiency in activity or level of ATM (Ataxia-Telangiectasia Mutated) and/or ATR (Ataxia Telangiectasia and Rad3-related protein); and/or
ii. if there is gain in activity or level of MDM2;
and
c) identifying the subject as likely to respond to the treatment with an MDM2 inhibitor based on: i) the deficiency in activity or level of ATM and/or ATR, or ii) the gain in activity or level of MDM2, or both i) and ii), found in the biological sample, or the method further comprising:
d) administering the MDM2 inhibitor to the subject identified as likely to respond to the treatment with an MDM2 inhibitor.

2. (canceled)

3. A method of treating a subject with cancer with an MDM2 inhibitor, the method comprising:
a) determining in a biological sample from the subject:
i. if there is deficiency in activity or level of ATM and/or ATR; and/or
ii. if there is gain in activity or level of MDM2; and
b) administering the subject with an MDM2 inhibitor based on: i) the deficiency in the activity or the level of ATM and/or ATR, or ii) the gain in activity or level of MDM2, or both i) and ii), found in the biological sample.

4. The method of claim 1, wherein the step of determining comprises detecting the presence of one or more inactivating mutation in ATM and/or ATR in the biological sample, wherein the presence of the inactivating mutation in ATM and/or ATR is indicative of the deficiency in activity or level of ATM and/or ATR, inactivating mutation comprises translocation, deletion, insertion, substitution, or any combination thereof, that reduces serine/threonine kinase activity of ATM and/or ATR, wherein the inactivating mutation in ATM comprises a mutation selected from the group of mutations relative to SEQ ID NO: 2 as listed in FIGS. 1B, 1C and 1D (e.g. H1380Y, N1983S, N2875S, R2598Q, 1599_1600del, V2716A, K1903fs, V2906I, A1127V, K1101E, Q912*, S2165F, H1083Y), or c.3154-2 A>G relative to SEQ ID NO: 1, or any combination thereof, wherein the inactivating mutation in ATR comprises K243T, Q1926H, I774fs, K1379N, L1483F, or any combination thereof, relative to SEQ ID NO: 4.

5-7. (canceled)

8. The method of claim 1, wherein the step of determining comprises:
determining if expression level of ATM and/or ATR is reduced in the biological sample relative to a reference level,
and wherein the reduced expression level of ATM and/or ATR is indicative of the deficiency in activity or level of ATM and/or ATR.

9. The method of claim 1, wherein the step of determining comprises:
determining if there is an increase in copy number variation of MDM2 gene, expression level of MDM2 gene product, or activity of MDM2 protein, in the biological sample relative to a reference level, and wherein the increase is indicative of the gain in activity or level of in MDM2, and/or wherein a copy number variation (CNV) of >3 in MDM2 is indicative of the gain in activity or level of MDM2, and/or wherein an increase of at least 50% in expression level of MDM2 gene product relative to the reference level as measured by RNAseg is indicative of gain in activity or level of MDM2.

10-11. (canceled)

12. The method of claim 1, wherein the step of identifying comprises:
c) identifying the subject as likely to respond to the treatment with an MDM2 inhibitor based on: both i) the presence of the inactivating mutation in ATM and/or ATR and ii) the increase in the CNV of the MDM2 gene or the increase in the expression level of the MDM2 gene product, found in the biological sample, or the step of determining further comprising: determining in the biological sample presence or absence of a functional p53, or the step of determining further comprising: determining in the biological sample if p53 is wild-type.

13-14. (canceled)

15. The method of claim 12, wherein the step of identifying comprises:
c) identifying the subject as likely to respond to the treatment with an MDM2 inhibitor based on: i) the presence of the inactivating mutation in ATM and/or ATR; ii) the increase in the CNV of the MDM2 gene or the increase in the expression level of the MDM2 gene product; and iii) presence of wild-type p53, found in the biological sample.

16. The method of claim 15, wherein i) the activity or level of ATM and/or ATR, or ii) the gain in activity or level of MDM2, or iii) the presence or absence of a functional p53, is measured by an amplification assay, a hybridization assay, a sequencing assay, or an immunoassay.

17. A method of treating a subject with cancer with an MDM2 inhibitor, wherein the subject has been identified as likely to respond to the treatment with the MDM2 inhibitor by the method of claim 1.

18. The method of claim 1, wherein the biological sample comprises a cancer cell or a non-cancer cell, and/or wherein the cancer is solid tumor or hematologic malignancy, and/or wherein the cancer is gastric cancer, cholangiocarcinoma, lung cancer, melanoma, breast cancer, colon cancer, ovarian cancer, prostate cancer, liver cancer (e.g. hepatocellular carcinoma), bladder cancer, pancreatic cancer, renal cancer, esophageal cancer, head and neck cancer, thyroid cancer, cutaneous squamous cell carcinoma, glioblastoma, neuroblastoma, urinary bladder cancer, hysterocarcinoma, melanoma, osteosarcoma, lymphoma (e.g., mantel cell lymphoma, diffuse large B cell lymphoma), leukemia (e.g., T-cell prolymphocytic leukemia, chronic lymphocytic leukemia, or acute myeloid leukemia), multiple myeloma, uterine cancer, colorectal cancer, lung adenocarcinoma, uterine carcinosarcoma CS, lung squamous cell carcinoma, cervical cancer, esophagus cancer, sarcoma, chromophobe, renal cell carcinoma (RCC), clear cell RCC, papillary RCC, uveal melanoma, testicular germ cell, low grade glioma (LGG), mesothelioma, pheochromocytoma and paraganglioma (PCPG), or thymoma.

19-20. (canceled)

21. The method of claim 18, wherein the cancer is gastric cancer.

22. The method of any claim 1, wherein the MDM2 inhibitor comprises a compound having the following formula (I): 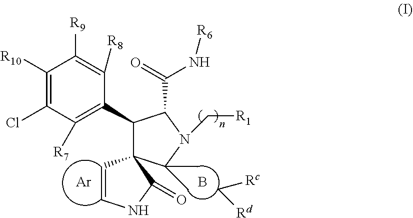
or a pharmaceutically acceptable salt thereof, wherein 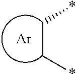
is selected from the group consisting of 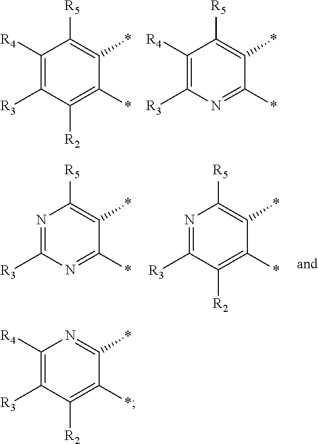
B is a C₄₋₇ carbocyclic ring;
R₁ is H, substituted or unsubstituted C₁₋₄ alkyl, substituted or unsubstituted cycloalkyl, substituted or unsubstituted heterocycloalkyl, OR^{a}, or NR^{a}R^{b};
n is 0, 1, or 2;
R₂, R₃, R₄, R₅, R₇, R₈, R₉, and R₁₀, independently, are selected from the group consisting of H, F, Cl, CH₃, and CF₃;
R₆ is 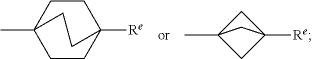
R^{a} is hydrogen or substituted or unsubstituted C₁₋₄ alkyl;
R^{b} is hydrogen or substituted or unsubstituted C₁₋₄ alkyl;
R^{c} and R^{d} are substituents on one carbon atom of ring B, wherein
R^{c} is H, C₁₋₃ alkyl, C₁₋₃ alkylene-OR^{a}, OR^{a}, or halo;
R^{d} is H, C₁₋₃ alkyl, C₁₋₃ alkylene-OR^{a}, OR^{a}, or halo; or
R^{c} and R^{d} are taken together with the carbon to which they are attached to form a 4 to 6-membered Spiro substituent, optionally containing an oxygen atom; and
R^{e} is C(═O)OR^{a}, C(═O)NR^{a}R^{b}, or C(═O)NHSO₂CH₃.

23-26. (canceled)

27. The method of claim 22, wherein the MDM2 inhibitor is a compound selected from: 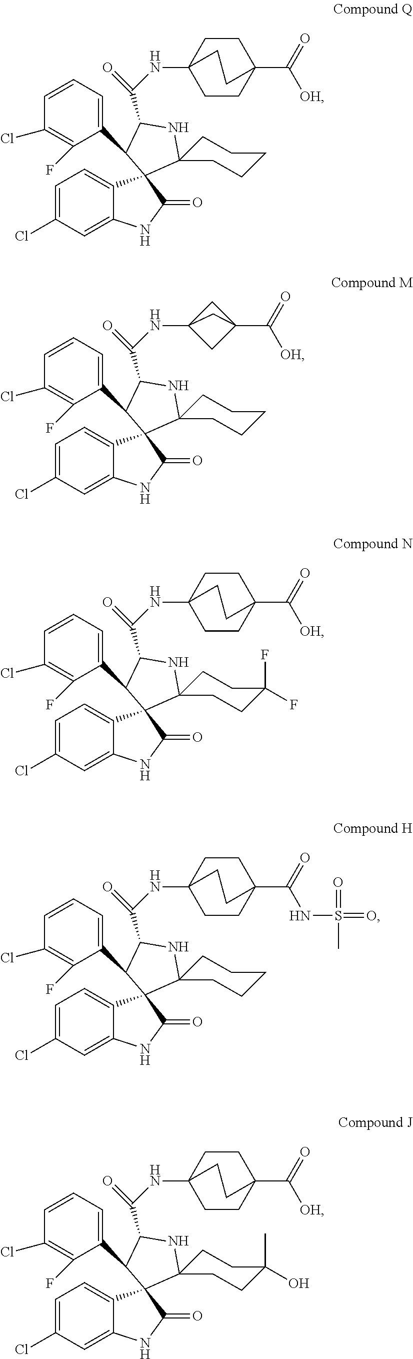 [SMILES: O=C(NC12CCC(C(=O)O)(CC1)CC2)[C@@H]1NC2(CCCCC2)[C@@]2(C(=O)Nc3cc(Cl)ccc32)[C@H]1c1cccc(Cl)c1F ; O=C(NC12CC(C(=O)O)(C1)C2)[C@@H]1NC2(CCCCC2)[C@@]2(C(=O)Nc3cc(Cl)ccc32)[C@H]1c1cccc(Cl)c1F ; O=C(NC12CCC(C(=O)O)(CC1)CC2)[C@@H]1NC2(CCC(F)(F)CC2)[C@@]2(C(=O)Nc3cc(Cl)ccc32)[C@H]1c1cccc(Cl)c1F ; CS(=O)(=O)NC(=O)C12CCC(NC(=O)[C@@H]3NC4(CCCCC4)[C@@]4(C(=O)Nc5cc(Cl)ccc54)[C@H]3c3cccc(Cl)c3F)(CC1)CC2 ; CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21] 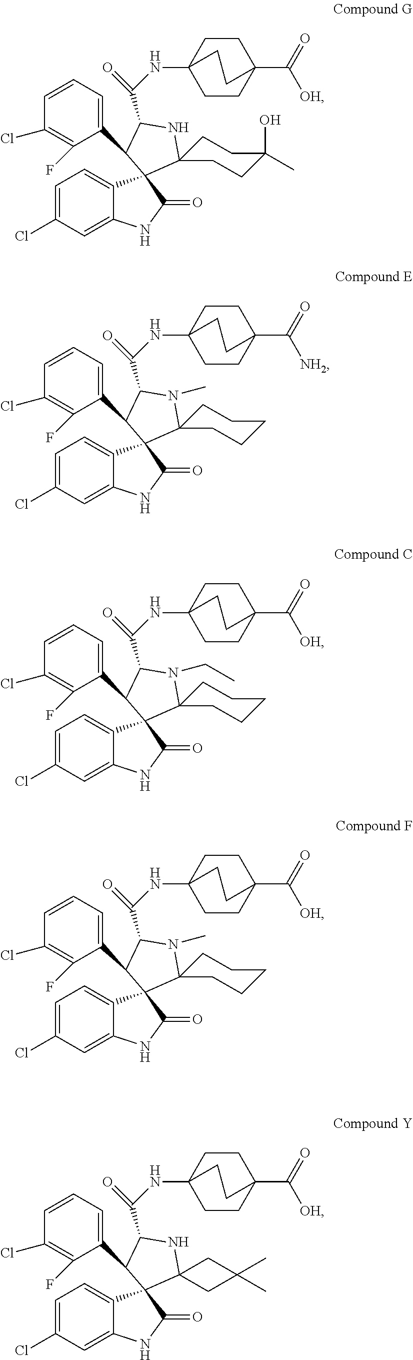 [SMILES: CC1(O)CCC2(CC1)N[C@@H](C(=O)NC13CCC(C(=O)O)(CC1)CC3)[C@H](c1cccc(Cl)c1F)[C@]21C(=O)Nc2cc(Cl)ccc21 ; CN1[C@@H](C(=O)NC23CCC(C(N)=O)(CC2)CC3)[C@H](c2cccc(Cl)c2F)[C@]2(C(=O)Nc3cc(Cl)ccc32)C12CCCCC2 ; CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2 ; CC1(C)CC2(C1)N[C@@H](C(=O)NC13CCC(C(=O)O)(CC1)CC3)[C@H](c1cccc(Cl)c1F)[C@]21C(=O)Nc2cc(Cl)ccc21] 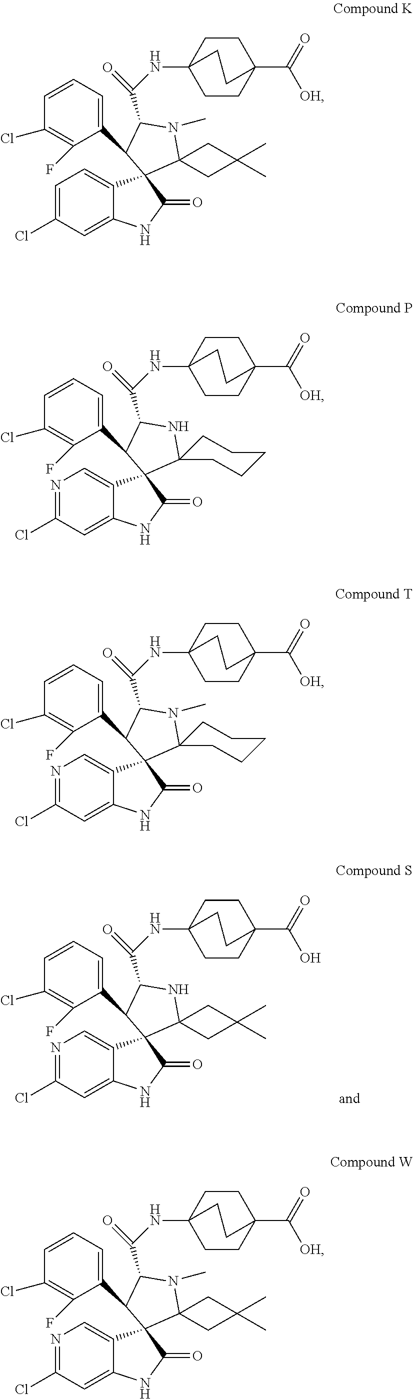 [SMILES: CN1[C@@H](C(=O)NC23CCC(C(=O)O)(CC2)CC3)[C@H](c2cccc(Cl)c2F)[C@]2(C(=O)Nc3cc(Cl)ccc32)C12CC(C)(C)C2 ; O=C(NC12CCC(C(=O)O)(CC1)CC2)[C@@H]1NC2(CCCCC2)[C@@]2(C(=O)Nc3cc(Cl)ncc32)[C@H]1c1cccc(Cl)c1F ; CN1[C@@H](C(=O)NC23CCC(C(=O)O)(CC2)CC3)[C@H](c2cccc(Cl)c2F)[C@]2(C(=O)Nc3cc(Cl)ncc32)C12CCCCC2 ; CC1(C)CC2(C1)N[C@@H](C(=O)NC13CCC(C(=O)O)(CC1)CC3)[C@H](c1cccc(Cl)c1F)[C@]21C(=O)Nc2cc(Cl)ncc21 ; CN1[C@@H](C(=O)NC23CCC(C(=O)O)(CC2)CC3)[C@H](c2cccc(Cl)c2F)[C@]2(C(=O)Nc3cc(Cl)ncc32)C12CC(C)(C)C2]
or a pharmaceutically acceptable salt of the compound and/or wherein the MDM2 inhibitor is Compound C or a pharmaceutically acceptable salt thereof.

28-29. (canceled)

30. The method of claim 1, wherein the method further comprises further administering an effective amount of one or more additional therapies comprising administering a modulator of an immune checkpoint molecule.

31. The method of claim 30, wherein the MDM2 inhibitor is Compound C or a pharmaceutically acceptable salt thereof and the modulator of an immune checkpoint molecule is an anti-PD-1 antibody.

32. A kit for predicting responsiveness of a subject with cancer to treatment with an MDM2 inhibitor, comprising:
a) one or more reagents for detecting presence of one or more inactivating mutations in ATM and/or ATR; or one or more reagents for measuring expression level of ATM and/or ATR; and/or
b) one or more reagents for measuring copy number variation of MDM2, or one or more reagents for measuring expression level of MDM2, or further comprising one or more reagents for detecting presence or absence of a functional p53.

33. (canceled)
